(19) United States
(12) Patent Application Publication
(10) Pub. No.: US 2026/0016381 A1
(43) Pub. Date: Jan. 15, 2026

(54) COMPOSITIONS AND METHODS FOR LYSING BLOOD SAMPLES COMPRISING ONE OR MORE INFECTIOUS AGENTS

(71) Applicant: Blastid, Inc., Boston, MA (US)

(72) Inventors: Thomas M. Feltrup, Weymouth, MA (US); Eduardo Luis Latouche, Chelsea, MA (US); Paulo Andres Garcia Dominguez, Somerville, MA (US)

(21) Appl. No.: 19/269,977

(22) Filed: Jul. 15, 2025

Related U.S. Application Data
(60) Provisional application No. 63/671,752, filed on Jul. 15, 2024.
(60) Provisional application No. 63/737,557, filed on Dec. 20, 2024.

(51) Int. Cl. G01N 1/30; C12Q 1/04

(52) U.S. Cl. CPC G01N 1/30; C12Q 1/04

(57) ABSTRACT

The present disclosure relates generally to compositions, systems, and methods for lysing blood samples comprising an infectious agent, for example a bacteria or a fungus, while maintaining viability of the infectious agent for subsequent characterization, for example phenotyping or genotyping. A lysing composition may include one or more surfactants, such as one or more detergents. A lysing composition may include lysing agent including one or more salts. A lysing composition may include one or more enzymes to break down one or more lyses byproducts, such as nucleic acids and/or protein aggregates.

### CROSS-REFERENCE TO RELATED APPLICATIONS

This application claims the benefit of U.S. Provisional Patent Application Nos. 63/671,752, filed on Jul. 15, 2024, and 63/737,557, filed on Dec. 20, 2024.

This invention was made with Government support under Agreement No. 1AY2AX000005-01, awarded by ARPA-H. The Government has certain rights in the invention.

### BACKGROUND

Bloodstream infections and sepsis remain among the leading causes of mortality worldwide. Sepsis contributes to approximately one-third of all hospital deaths. The high mortality rate is mostly associated with the challenges of diagnosing sepsis in a timely and accurate manner. Current diagnostic workflows are hindered by limitations in the sample preparation process. Microbial cells, such as bacteria and fungi, are typically present at low concentrations in fluids, making detection challenging. For example, traditional blood culture often takes approximately 3-5 days of growth incubation to become positive due to the low concentration of microbial cells in the bloodstream, thus delaying identification of infection sources and critical intervention. Some diagnostic techniques for detecting bacteria in blood are often time consuming and may be ineffective at isolating bacteria. In addition, conventional diagnostic techniques for isolating microbial cells may require pre-processing steps such as centrifugation, which may further restrict the ability to process samples efficiently and in a timely manner which is required to achieve the most meaningful impact to patient outcome. As a result, there is an urgent need for improved sample preparation and analysis methods in the management of bacterial infections and sepsis.

### SUMMARY

The subject matter of the present disclosure involves, in some cases, compositions, systems, and methods for lysing blood samples comprising an infectious agent. Methods disclosed herein are directed to an effective workflow for preparing (e.g., whole) blood samples (e.g., for use with a microfluidics device) comprising one or more infectious agents (e.g., one or more bacteria, one or more fungi, one or more prokaryotic cell, one or more eukaryotic cell, one or more viruses, or a combination thereof).

Methods disclosed herein provide, inter alia, an effective workflow for preparing (e.g., whole) blood samples (e.g., for use with a microfluidics device) which includes lysis of intact blood cells while maintaining viability of bacteria cells in the sample. Methods disclosed herein can be used in combination with a diagnostic for detecting bacteria in blood to be able to provide medical practitioners with actionable information for appropriate clinical regime. Patients being impacted by bacterial infection, in particular bacteremia and/or sepsis, have urgency to arrive at the right treatment fast in order to reduce complications and/or improve mortality rates. Rapid processing is a critical requirement for every step in a workflow for sample preparation and analysis. Methods disclosed herein may improve the sensitivity, speed, and accuracy of diagnostics, and may also expand access to testing and treatment in a variety of clinical settings, especially in resource-limited environments.

Disclosed herein are, inter alia, methods to selectively lyse blood cells while maintaining viability of one or more infectious agents [e.g., one or more bacteria, one or more fungi, one or more prokaryotic cell, one or more eukaryotic cell, one or more viruses (e.g., bound to particles), or a combination thereof] in the samples (e.g., without destroying or harming the cells of the infectious agent and/or while maintaining infectious agent cell viability). Maximizing the lysing of blood cells while preserving viability of infectious agents may be beneficial for downstream analysis, such as phenotyping (e.g., by further imaging, detecting, observing, and monitoring) the infectious agent. Certain analyses, such as phenotyping, may be sensitive to the current state of an infectious agent. Harmful lysing compositions may destroy or degrade an infectious agent such that such analysis, or analyses, may not be accurately performed. For example, harmful lysing compositions may alter phenotype of an infectious agent during lysing such that the infectious agent may be difficult or impossible to accurately identify from a lysed blood composition, especially using imaging-based methods, for example label-free microscopy techniques. Methods and compositions disclosed herein may preserve an infectious agent during lysing such that the analysis, or analyses, may be accurately performed, for example so that an infectious agent may be accurately phenotyped, such as with an imaging-based method, for example a label-free microscopy technique. Methods may be used to prepare samples for use with a microfluidics device, for example for clinical diagnostic applications.

Disclosed herein are, inter alia, methods to selectively concentrate bacteria cells in whole blood samples before being treated with lysis buffer formulations which will quickly, and effectively, lyse red blood cells in whole blood without destroying or harming bacteria cells and/or while maintaining bacteria cell viability. Methods may be used to prepare samples for use with a microfluidics device, for example for clinical diagnostic applications.

In some embodiments, bacteria in a (e.g., whole) blood sample can be concentrated through centrifugation by centrifuging sample at higher speeds (e.g., 4,000 rotations per minute (rpm)) for a period of time (e.g., 10-15 min) to collect all blood cells and bacteria (e.g., from the lowest layer in the centrifuged sample). In some embodiments, bacteria in a (e.g., whole) blood sample can be concentrated through centrifugation by centrifuging sample at lower speeds (e.g., 500 rpm) for a period of time (e.g., 10-15 min) to concentrate blood cells in the lowest layer in the sample while the bulk of the bacteria remain in the supernatant layer.

In some embodiments, blood cells, particularly red blood cells (RBC), can be lysed through chemical treatment to prepare a blood sample for injection onto or into a microfluidics device. In some embodiments, a microfluidics device requires minimal intact RBC in samples to prevent clogs, blocking of trenches, and/or decreased capture of bacteria in the microfluidics device. Whole blood samples may be treated with a lysing composition (e.g., a lysis buffer composition) (e.g., comprising lysing agent, such as, for example, Triton X-100, IGEPAL CA-630, sodium cholate, or saponin) prepared in either phosphate buffered saline (PBS) or water, for example at concentrations up to 5% (e.g., by volume). A lysing composition may or may not include an additional lysing agent (e.g. additional detergent or surfactant) (e.g., ammonium chloride and/or sodium chloride, Pluronic F-127, Pluronic F-128, and/or Tween 20) to further improve the lysis of RBC and/or lysed blood composition viscosity. In some embodiments, a lysing composition comprises a surfactant. Using saponin as a surfactant in a lysing composition contacted to a blood sample, in particular fresh whole blood, may be less damaging to an infectious agent (e.g., a bacteria or fungus), which may be advantageous for maintaining viability and recovery of the infectious agent for downstream analysis (e.g., using a microfluidics device) and/or further culturing. In comparison, it has been observed that some surfactants, such as Triton X-100 and/or IGEPAL CA-630, while being effective at lysing blood cells, may also result in more bacterial cell disruption compared to saponin. Thus, using a combination of multiple surfactants may improve lysing of RBCs (e.g., RBC lysis percentage), while maintaining sample viability for further analysis (e.g., phenotyping of the sample). In general, in some embodiments, the extent of lysis needed will be dependent on requirements for a microfluidics device. Current data, presented herein, has demonstrated 85+% lysis achieved with a 1% concentration of lysis agent in a composition of blood sample and lysing composition with increasing percent lysis at higher concentrations of lysing composition (e.g., with only 5 minutes of exposure time). Lysing compositions can be prepared at high stock solution (e.g. 25% or higher), for example to minimize the dilution of the sample and maintain the smallest volume possible, for example for injection onto or into a microfluidics device. Minimizing total volume for a lysed blood composition is generally preferable for microfluidic applications that utilize small volumes. Diluting blood samples with large volumes of lysing composition in order to lyse blood cells (e.g., RBCs) is generally undesirable for microfluidic applications. However, methods disclosed herein also comprise, inter alia, combinations (e.g., mixtures) of blood samples and lysing compositions at high lysing composition to blood sample ratio. Using a high ratio of lysing composition to blood sample may enable effective lysis even when a lower volume of blood is available, which is crucial when sample is limited. Alternatively or additionally, this approach may facilitate processing the same or higher blood volume per unit time as compared with using a low lysing composition to blood sample ratio, which may improve the speed of diagnostics. A microfluidics device may be used to culture bacteria cells from a sample and/or performing antimicrobial susceptibility testing (AST) and/or identifying an identity (e.g., strain) (e.g., genus) (e.g., type) of bacteria. Quantitative phase microscopy (also called quantitative phase-contrast microscopy) and/or hyperspectral imaging may be used as part of AST and/or bacteria identification.

In some embodiments, a lysing composition comprises one or more enzymes. One or more enzymes may be used to break down lysis byproducts. One or more enzymes may be used in a lysis composition to degrade nucleic acids (e.g., DNA, RNA) and/or protein aggregates. In some embodiments, a lysing composition comprises one or more cofactors for one or more enzymes in the lysing composition.

Lysing compositions disclosed herein (e.g., and evaluated) may maintain bacteria viability in blood samples (e.g., whole blood samples), for example such that the bacteria can subsequently be cultured (e.g., using a microfluidics device). Lysing compositions disclosed herein (e.g., and evaluated) may keep bacteria in blood samples (e.g., whole blood samples) intact. Bacteria in blood samples (e.g., whole blood samples) contacted with lysing compositions disclosed herein (e.g., and evaluated) may survive lysing of blood cells (e.g., RBCs). Lysing compositions disclosed herein (e.g., and evaluated) minimally impact growth curve of bacteria cells, for example such lysing does not delay bacteria assay turnaround time. Methods disclosed herein may prepare a blood sample comprising bacteria, e.g. for culturing of the bacteria, without delaying bacteria assay turnaround time.

Methods disclosed herein provide, inter alia, fast and effective workflows for preparing a (e.g., whole) blood sample (e.g., for use with a microfluidics device) while maintaining viability (e.g., integrity) of bacteria in the sample. The ability to maximize the number of live bacteria allows for faster capture, culturing, identification, and/or susceptibility testing, for example on a microfluidics device, for example to inform medical practitioners.

The ability to rapidly and cost-effectively prepare samples from blood, especially whole blood, allows for diagnostic applications, especially those to be deployed in clinical settings and also in geographies in which there are limited infrastructure. This ranges from, for example, nursing homes, family doctors' office, battle fields, community hospitals and rural areas which significantly expands the commercial opportunity and deployment of diagnostics that use compositions and/or methods disclosed herein. Of particular importance is that certain compositions and methods disclosed herein do not required the use of centrifugation in order to sufficiently lyse blood cells and/or preserve infectious agent(s). The present disclosure recognizes that there is a long-felt need to avoid centrifugation, which is notoriously known for infectious agent (e.g., pathogen) loss during methods of lysing blood samples comprising one or more infectious agents (e.g., one or more bacteria and/or one or more fungi) that can be addressed by using methods disclosed herein, which can sufficiently lyse blood cells while preserving infectious agent(s) without the use of centrifugation.

A microfluidics device may be a consumable device, for example, used for a single test, used a single time, and/or used for a single patient.

Any two or more of the features described in this specification, including in this summary section, may be combined to form implementations of the disclosure, whether specifically expressly described as a separate combination in this specification or not.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way. The foregoing and other objects, aspects, features, and advantages of the disclosure will become more apparent and may be better understood by referring to the following description taken in conjunction with the accompanying drawings.

FIG. 1A shows a schematic that outlines properties for whole blood cell preparation, according to illustrative embodiments of the present disclosure.

FIG. 1B shows a picture of a microfluidic system for bacterial analysis, according to illustrative embodiments of the present disclosure.

FIG. 1C shows a microscopic image of bacteria in a microfluidic system, according to illustrative embodiments of the present disclosure.

FIG. 1D shows a schematic of a microfluidic system for bacterial analysis, according to illustrative embodiments of the present disclosure.

FIG. 2A shows a box plot of red blood cells (RBC) lysis percentage for frozen ethylenediaminetetraacetic acid (EDTA) whole blood samples treated for different times with different lysis buffers at 1% concentration in water, according to illustrative embodiments of the present disclosure.

FIG. 2B shows a box plot of RBC lysis percentage for fresh EDTA whole blood samples treated for different times with different lysis buffers at either 1% or 2.5% concentration in water, according to illustrative embodiments of the present disclosure.

FIG. 2C shows a box plot of RBC lysis percentage for fresh EDTA whole blood samples treated for 5 min with different lysis buffers at either 1% or 2.5% concentration in water, according to illustrative embodiments of the present disclosure.

FIG. 3 shows a box plot of RBC lysis percentage for frozen EDTA whole blood samples treated with different lysis buffers during sample rocking or vortex mixing, according to illustrative embodiments of the present disclosure.

FIG. 4 shows a box plot of RBC lysis percentage for frozen EDTA whole blood samples treated for 5 minutes with different lysis buffers at different concentrations in water, according to illustrative embodiments of the present disclosure.

FIG. 5A shows a box plot of RBC lysis percentage for frozen EDTA whole blood samples treated with saponin buffer at different concentrations in water with and without 300 mM ammonium chloride, according to illustrative embodiments of the present disclosure.

FIG. 5B shows a box plot of RBC lysis percentage for frozen EDTA whole blood samples treated with Triton X-100 buffer at different concentrations in water with and without 300 mM ammonium chloride, according to illustrative embodiments of the present disclosure.

FIG. 5C shows a box plot of RBC lysis percentage for frozen EDTA whole blood samples treated with sodium cholate buffer at different concentrations in water with and without 300 mM ammonium chloride, according to illustrative embodiments of the present disclosure.

FIG. 5D shows a box plot of RBC lysis percentage for frozen EDTA whole blood samples treated with IGEPAL CA-630 buffer at different concentrations in water with and without 300 mM ammonium chloride, according to illustrative embodiments of the present disclosure.

FIG. 6A shows a box plot of RBC lysis percentage for frozen EDTA whole blood samples of various volumes treated with different lysis buffers at 1% concentrations in water, according to illustrative embodiments of the present disclosure.

FIG. 6B shows a box plot of RBC lysis percentage for frozen EDTA whole blood samples of various volumes treated with different lysis buffers at 1% concentrations in water, according to illustrative embodiments of the present disclosure.

FIG. 7 shows a box plot of RBC lysis percentage for fresh EDTA whole blood samples at various dilutions treated with different lysis buffers at 1% concentrations in water, according to illustrative embodiments of the present disclosure.

FIG. 8A shows a box plot of colony-forming unit (CFU) recovery percentage for E. coli K-12 (ATCC 10798) samples treated with different lysis buffers at 1% concentrations in water, according to illustrative embodiments of the present disclosure.

FIG. 8B shows a box plot of colony-forming unit (CFU) recovery percentage for E. coli K-12 (ATCC 10798) samples treated with different lysis buffers at 1% concentrations in water, according to illustrative embodiments of the present disclosure.

FIG. 8C shows a box plot of colony-forming unit (CFU) recovery percentage for E. coli K-12 (ATCC 10798) samples exposed 5 minutes to different lysis buffers at 1% concentrations in water, according to illustrative embodiments of the present disclosure.

FIG. 9A shows a box plot of colony-forming unit (CFU) recovery percentage for different bacteria strain samples exposed 5 minutes to DNase I, according to illustrative embodiments of the present disclosure.

FIG. 9B shows a box plot of colony-forming unit (CFU) recovery percentage for different bacteria strain samples exposed 5 minutes to Proteinase K, according to illustrative embodiments of the present disclosure.

FIG. 9C shows a box plot of colony-forming unit (CFU) recovery percentage for different bacteria strain samples exposed 5 minutes to Lipase, according to illustrative embodiments of the present disclosure.

FIG. 9D shows a box plot of colony-forming unit (CFU) recovery percentage for different bacteria strain samples exposed 15 minutes to different poloxamers at different concentrations in phosphate buffered saline (PBS), according to illustrative embodiments of the present disclosure.

FIG. 9E shows a box plot of colony-forming unit (CFU) recovery percentage for different Candida strain samples exposed 5 minutes to DNase I, according to illustrative embodiments of the present disclosure.

FIG. 9F shows a box plot of colony-forming unit (CFU) recovery percentage for different Candida strain samples exposed 5 minutes to Proteinase K, according to illustrative embodiments of the present disclosure.

FIG. 9G shows a box plot of colony-forming unit (CFU) recovery percentage for different Candida strain samples exposed 5 minutes to Lipase, according to illustrative embodiments of the present disclosure.

FIG. 9H shows a box plot of colony-forming unit (CFU) recovery percentage for different Candida strain samples exposed 15 minutes to different poloxamers at different concentrations in PBS, according to illustrative embodiments of the present disclosure.

FIG. 10A shows a box plot of colony-forming unit (CFU) recovery percentage for different bacteria strain samples exposed for 5 minutes to saponin lysis buffers at 1% concentration in PBS or bacteria specific growth media, according to illustrative embodiments of the present disclosure.

FIG. 10B shows a box plot of colony-forming unit (CFU) recovery percentage for different bacteria strain samples exposed for 5 minutes to ammonium chloride lysis buffers at 150 mM and 300 mM concentrations in PBS or bacteria specific growth media, according to illustrative embodiments of the present disclosure.

FIG. 10C shows a box plots of colony-forming unit (CFU) recovery percentage for different Candida strain samples exposed for 5 minutes to saponin lysis buffers at 1% concentration in PBS or YM growth media, according to illustrative embodiments of the present disclosure.

FIG. 10D shows a box plots of colony-forming unit (CFU) recovery percentage for different Candida strain samples exposed for 5 minutes to Triton X-100 lysis buffers in PBS or YM growth media, according to illustrative embodiments of the present disclosure.

FIG. 10E shows a box plots of colony-forming unit (CFU) recovery percentage for different Candida strain samples exposed for 5 minutes to ammonium chloride lysis buffers at 150 mM and 300 mM concentrations in PBS or YM growth media, according to illustrative embodiments of the present disclosure.

FIG. 10F shows a box plot of colony-forming unit (CFU) recovery percentage for different Candida strain samples exposed for 60 minutes to saponin lysis buffers at 1% concentration in PBS or YM growth media, according to illustrative embodiments of the present disclosure.

FIG. 10G shows a box plot of colony-forming unit (CFU) recovery percentage for different Candida strain samples exposed for 60 minutes to Triton X-100 lysis buffers in PBS or YM growth media, according to illustrative embodiments of the present disclosure.

FIG. 10H shows a box plot of colony-forming unit (CFU) recovery percentage for different Candida strain samples exposed for 60 minutes to ammonium chloride lysis buffers at 150 mM and 300 mM concentrations in PBS or YM growth media, according to illustrative embodiments of the present disclosure.

FIG. 11A shows a photograph of a centrifugal tube after centrifugation containing freshly drawn EDTA whole blood with a pellet and a supernatant clearly visible, according to illustrative embodiments of the present disclosure.

FIG. 11B shows a box plot of RBC total count for fresh EDTA whole blood samples with different centrifugation conditions, according to illustrative embodiments of the present disclosure.

FIG. 11C shows a box plot of estimated colony-forming unit (CFU) concentrations for E. coli K-12 (ATCC 10798) samples mixed with different samples of fresh EDTA whole blood after various centrifugation conditions, according to illustrative embodiments of the present disclosure.

FIG. 12 shows a box plot of colony-forming unit (CFU) recovery percentage for different bacteria strain samples exposed 30 minutes to a lysing composition at a dilution ratio of 1:10 at 37° C., according to illustrative embodiments of the present disclosure.

FIG. 13 shows a box plot of colony-forming unit (CFU) recovery percentage for different Candida strain samples exposed 15 minutes to a lysing composition at a dilution ratio of 1:5 at room temperature, according to illustrative embodiments of the present disclosure.

FIG. 14 shows a box plot of RBC lysis percentage for sodium polyanethole sulfonate (SPS) whole blood samples exposed 30 minutes at 37° C. to a lysing composition, according to illustrative embodiments of the present disclosure.

### DETAILED DESCRIPTION OF CERTAIN EMBODIMENTS

In some embodiments, a method includes contacting (e.g., combining) (e.g., mixing) a blood sample comprising one or more infectious agents to a lysing composition thereby obtaining a lysed blood composition comprising the one or more infectious agents. An infectious agent may be a pathogen. An infectious agent may be, for example, a virus (e.g., bound to particles), a bacteria, a fungus, a prokaryotic cell, a eukaryotic cell, or a combination thereof. A pathogen may be, for example, a virus (e.g., bound to particles), a bacteria, a fungus, a prokaryotic cell, a eukaryotic cell, or a combination thereof. A blood sample may include a plurality of infectious agents. In some embodiments, a method includes contacting (e.g., combining) (e.g., mixing) a blood sample comprising bacteria to a lysing composition thereby obtaining a lysed blood composition comprising the bacteria. In some embodiments, a method includes contacting (e.g., combining) (e.g., mixing) a blood sample comprising one or more bacteria to a lysing composition thereby obtaining a lysed blood composition comprising the one or more bacteria. In some embodiments, a method includes contacting (e.g., combining) (e.g., mixing) a blood sample comprising one or more fungi to a lysing composition thereby obtaining a lysed blood composition comprising the one or more fungi. In some embodiments, a method includes contacting (e.g., combining) (e.g., mixing) a blood sample comprising one or more prokaryotic cells to a lysing composition thereby obtaining a lysed blood composition comprising the one or more prokaryotic cells. In some embodiments, a method includes contacting (e.g., combining) (e.g., mixing) a blood sample comprising one or more eukaryotic cells to a lysing composition thereby obtaining a lysed blood composition comprising the one or more eukaryotic cells. In some embodiments, a method includes contacting (e.g., combining) (e.g., mixing) a blood sample comprising one or more viruses (e.g., bound to particles) to a lysing composition thereby obtaining a lysed blood composition comprising the one or more viruses.

In some embodiments, a lysing composition is a lysis buffer. In some embodiments, a lysing composition comprises a lysis buffer composition. In some embodiments, a lysing composition comprises lysing agent. In some embodiments, lysing agent comprises (e.g., is) a detergent. In some embodiments, lysing agent comprises (e.g., is) a surfactant. In some embodiments, lysing agent comprises (e.g., is) one or more nonionic agents (e.g., a nonionic surfactant). In some embodiments, lysing agent comprises (e.g., is) a non-detergent surfactant. In some embodiments, lysing agent comprises (e.g., is) one or more non-denaturing agents. In some embodiments, lysing agent comprises (e.g., is) one or more salts.

In some embodiments, lysing agent comprises (e.g., is) Triton X-100. In some embodiments, lysing agent comprises (e.g., is) IGEPAL CA-630. In some embodiments, lysing agent comprises (e.g., is) one or more saponins. In some embodiments, lysing agent comprises (e.g., is) Tween 20. In some embodiments, lysing agent comprises (e.g., is) Tween 80. In some embodiments, lysing agent comprises (e.g., is) sodium cholate. In some embodiments, lysing agent comprises (e.g., is) one or more poloxamers (e.g., Poloxamer 407, Pluronic F-127, Pluronic F-108, Pluronic F-68). In some embodiments, lysing agent comprises (e.g., is) Pluronic F-127. In some embodiments, lysing agent comprises (e.g., further comprises) ammonium chloride and/or sodium chloride.

In some embodiments, a concentration of lysing agent in a lysing composition is no more than 10% (e.g., by volume). In some embodiments, a concentration of lysing agent in a lysing composition is no more than 5% (e.g., by volume). In some embodiments, a concentration of lysing agent in a lysing composition is no more than 4% (e.g., by volume). In some embodiments, a concentration of lysing agent in a lysing composition is no more than 3% (e.g., by volume). In some embodiments, a concentration of lysing agent in a lysing composition is no more than 2% (e.g., by volume). In some embodiments, a concentration of lysing agent in a lysing composition is no more than 1% (e.g., by volume). In some embodiments, a concentration of lysing agent in a lysing composition is in a range of from 0.1% to 3% (e.g., by volume). In some embodiments, a concentration of lysing agent in a lysing composition is in a range of from 0.5% to 3% (e.g., by volume). In some embodiments, a concentration of lysing agent in a lysing composition is in a range of from 0.1% to 2% (e.g., by volume). In some embodiments, a concentration of lysing agent in a lysing composition is in a range of from 0.5% to 2% (e.g., by volume). In some embodiments, a concentration of lysing agent in a lysing composition is in a range of from 0.1% to 1.5% (e.g., by volume). In some embodiments, a concentration of lysing agent in a lysing composition is in a range of from 0.25% to 1.5% (e.g., by volume). In some embodiments, a concentration of lysing agent in a lysing composition is in a range of from 0.5% to 1.5% (e.g., by volume). In some embodiments, a concentration of lysing agent in a lysing composition is in a range of from 0.1% to 1% (e.g., by volume). In some embodiments, a concentration of lysing agent in a lysing composition is in a range of from 0.5% to 1% (e.g., by volume). In some embodiments, a concentration of lysing agent in a lysing composition is at least 10% (e.g., by volume). In some embodiments, a concentration of lysing agent in a lysing composition is at least 15% (e.g., by volume). In some embodiments, a concentration of lysing agent in a lysing composition is at least 20% (e.g., by volume). In some embodiments, a concentration of lysing agent in a lysing composition is at least 25% (e.g., by volume). In some embodiments, a concentration of lysing agent in a lysing composition is no more than 50% (e.g., by volume).

In some embodiments, a concentration of lysing agent in a total volume of a lysing composition and a blood sample when the lysing composition and the blood sample are first contacted is at least 0.1% and no more than 3% (e.g., by volume). In some embodiments, a concentration of lysing agent in a total volume of a lysing composition and a blood sample when the lysing composition and the blood sample are first contacted is at least 0.5% and no more than 3% (e.g., by volume). In some embodiments, a concentration of lysing agent in a total volume of a lysing composition and a blood sample when the lysing composition and the blood sample are first contacted is at least 0.4% and no more than 3% (e.g., by volume). In some embodiments, a concentration of lysing agent in a total volume of a lysing composition and a blood sample when the lysing composition and the blood sample are first contacted is at least 0.1% and no more than 2% (e.g., by volume). In some embodiments, a concentration of lysing agent in a total volume of a lysing composition and a blood sample when the lysing composition and the blood sample are first contacted is at least 0.5% and no more than 2% (e.g., by volume). In some embodiments, a concentration of lysing agent in a total volume of a lysing composition and a blood sample when the lysing composition and the blood sample are first contacted is at least 0.1% and no more than 1.5% (e.g., by volume). In some embodiments, a concentration of lysing agent in a total volume of a lysing composition and a blood sample when the lysing composition and the blood sample are first contacted is at least 0.25% and no more than 3% (e.g., by volume). In some embodiments, a concentration of lysing agent in a total volume of a lysing composition and a blood sample when the lysing composition and the blood sample are first contacted is at least 0.5% and no more than 1.5% (e.g., by volume). In some embodiments, a concentration of lysing agent in a total volume of a lysing composition and a blood sample when the lysing composition and the blood sample are first contacted is at least 0.1% and no more than 1% (e.g., by volume). In some embodiments, a concentration of lysing agent in a total volume of a lysing composition and a blood sample when the lysing composition and the blood sample are first contacted is at least 0.75% and no more than 1.25% (e.g., by volume). In some embodiments, a concentration of lysing agent in a total volume of a lysing composition and a blood sample when the lysing composition and the blood sample are first contacted is at least 0.5% and no more than 1% (e.g., by volume). In some embodiments, a concentration of lysing agent in a total volume of a lysing composition and a blood sample when the lysing composition and the blood sample are first contacted is about 1% (e.g., is 1%) (e.g., by volume). In some embodiments, a concentration of lysing agent in a total volume of a lysing composition and a blood sample when the lysing composition and the blood sample are first contacted is in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%). In some embodiments, a concentration of lysing agent in a total volume of a lysing composition and a blood sample when the lysing composition and the blood sample are first contacted is in a range of from 0.01% to 0.3% (e.g., from 0.01% to 0.2%, from 0.01% to 0.1%, from 0.025% to 0.2%, or from 0.025% to 0.1%). In some embodiments, a concentration of lysing agent in a total volume of a lysing composition and a blood sample when the lysing composition and the blood sample are first contacted is in a range of from 0.4% to 5% (e.g., from 0.4% to 2%, from 0.4% to 1%, from 0.4% to 0.8%, or from 0.4% to 0.6%).

In some embodiments, a concentration of lysing agent in a lysed blood composition is at least 0.1% and no more than 3% (e.g., by volume). In some embodiments, a concentration of lysing agent in a lysed blood composition is at least 0.5% and no more than 3% (e.g., by volume). In some embodiments, a concentration of lysing agent in a lysed blood composition is at least 0.4% and no more than 3% (e.g., by volume). In some embodiments, a concentration of lysing agent in a lysed blood composition is at least 0.4% and no more than 3% (e.g., by volume). In some embodiments, a concentration of lysing agent in a lysed blood composition is at least 0.1% and no more than 2% (e.g., by volume). In some embodiments, a concentration of lysing agent in a lysed blood composition is at least 0.5% and no more than 2% (e.g., by volume). In some embodiments, a concentration of lysing agent in a lysed blood composition is at least 0.1% and no more than 1.5% (e.g., by volume). In some embodiments, a concentration of lysing agent in a lysed blood composition is at least 0.25% and no more than 3% (e.g., by volume). In some embodiments, a concentration of lysing agent in a lysed blood composition is at least 0.5% and no more than 1.5% (e.g., by volume). In some embodiments, a concentration of lysing agent in a lysed blood composition is at least 0.1% and no more than 1% (e.g., by volume). In some embodiments, a concentration of lysing agent in a lysed blood composition is at least 0.75% and no more than 1.25% (e.g., by volume). In some embodiments, a concentration of lysing agent in a lysed blood composition is at least 0.5% and no more than 1% (e.g., by volume). In some embodiments, a concentration of lysing agent in a lysed blood composition is about 1% (e.g., is 1%) (e.g., by volume).

In some embodiments, a lysing composition comprises (e.g., further comprises) ammonium chloride and/or sodium chloride. In some embodiments, a lysing composition comprises water. In some embodiments, a lysing composition comprises phosphate buffered saline.

A lysing composition may comprise growth media, which may be used to cultivate and/or propagate growth of an infectious agent (e.g., a bacteria or fungus) for the lysis process. In some embodiments, a lysing composition comprises growth media. In some embodiments, growth media comprises (e.g., is) tryptic soy broth. In some embodiments, growth media comprises (e.g., is) comprises Mueller Hinton broth. In some embodiments, growth media comprises (e.g., is) comprises cation-adjusted Mueller Hinton broth. In some embodiments, growth media comprises (e.g.,) is LB broth. In some embodiments, growth media comprises (e.g., is) Nutrient broth. In some embodiments, growth media comprises (e.g.,) is Brain Heart Infusion broth. In some embodiments, growth media comprises (e.g., is) comprises YM broth.

In some embodiments, a lysing composition comprises ammonium chloride and/or sodium chloride. In some embodiments, a concentration of ammonium chloride and/or a concentration of sodium chloride in a total volume of a lysing composition and a blood sample when the lysing composition and the blood sample are first contacted is no more than 500 mM (e.g., from 10 mM to 500 mM, from 50 mM to 500 mM, from 100 mM to 500 mM, from 100 mM to 400 mM, from 150 mM to 350 mM, from 100 mM to 300 mM, from 100 mM to 200 mM, from 200 mM to 500 mM, from 200 mM to 400 mM) (e.g., in water, phosphate buffered saline, or growth media). In some embodiments, a concentration of ammonium chloride and/or a concentration of sodium chloride in a total volume of a lysing composition and a blood sample when the lysing composition and the blood sample are first contacted is at least 100 mM and no more than 500 mM. In some embodiments, a concentration of ammonium chloride and/or a concentration of sodium chloride in a total volume of a lysing composition and a blood sample when the lysing composition and the blood sample are first contacted is at least 100 mM and no more than 400 mM. In some embodiments, a concentration of ammonium chloride and/or a concentration of sodium chloride in a total volume of a lysing composition and a blood sample when the lysing composition and the blood sample are first contacted is at least 100 mM and no more than 350 mM. In some embodiments, a concentration of ammonium chloride and/or a concentration of sodium chloride in a total volume of a lysing composition and a blood sample when the lysing composition and the blood sample are first contacted is at least 100 mM and no more than 300 mM. In some embodiments, a concentration of ammonium chloride and/or a concentration of sodium chloride in a total volume of a lysing composition and a blood sample when the lysing composition and the blood sample are first contacted is at least 200 mM and no more than 400 mM. In some embodiments, a concentration of ammonium chloride and/or a concentration of sodium chloride in a total volume of a lysing composition and a blood sample when the lysing composition and the blood sample are first contacted is at least 150 mM and no more than 300 mM.

In some embodiments, a lysing composition comprises one or more surfactants. In some embodiments, a lysing composition comprises one or more nonionic surfactants. In some embodiments, a lysing composition comprises one or more non-detergent surfactants.

In some embodiments, a concentration of each of one or more surfactants in a lysing composition is no more than 10% [e.g., by volume (e.g., by weight per volume or by volume per volume)]. In some embodiments, a concentration of each of one or more surfactants in a lysing composition is no more than 5% [e.g., by volume (e.g., by weight per volume or by volume per volume)]. In some embodiments, a concentration of each of one or more surfactants in a lysing composition is no more than 4% [e.g., by volume (e.g., by weight per volume or by volume per volume)]. In some embodiments, a concentration of each of one or more surfactants in a lysing composition is no more than 3% [e.g., by volume (e.g., by weight per volume or by volume per volume)]. In some embodiments, a concentration of each of one or more surfactants in a lysing composition is no more than 2% [e.g., by volume (e.g., by weight per volume or by volume per volume)]. In some embodiments, a concentration of each of one or more surfactants in a lysing composition is no more than 1% [e.g., by volume (e.g., by weight per volume or by volume per volume)]. In some embodiments, a concentration of each of one or more surfactants in a lysing composition is no more than 0.5% [e.g., by volume (e.g., by weight per volume or by volume per volume)]. In some embodiments, a concentration of each of one or more surfactants in a lysing composition is in a range of from 0.01% to 3% [e.g., by volume (e.g., by weight per volume or by volume per volume)]. In some embodiments, a concentration of each of one or more surfactants in a lysing composition is in a range of from 0.025% to 3% [e.g., by volume (e.g., by weight per volume or by volume per volume)]. In some embodiments, a concentration of each of one or more surfactants in a lysing composition is in a range of from 0.05% to 3% [e.g., by volume (e.g., by weight per volume or by volume per volume)]. In some embodiments, a concentration of each of one or more surfactants in a lysing composition is in a range of from 0.1% to 3% [e.g., by volume (e.g., by weight per volume or by volume per volume)]. In some embodiments, a concentration of each of one or more surfactants in a lysing composition is in a range of from 0.5% to 3% [e.g., by volume (e.g., by weight per volume or by volume per volume)]. In some embodiments, a concentration of each of one or more surfactants in a lysing composition is in a range of from 0.01% to 2% [e.g., by volume (e.g., by weight per volume or by volume per volume)]. In some embodiments, a concentration of each of one or more surfactants in a lysing composition is in a range of from 0.025% to 2% [e.g., by volume (e.g., by weight per volume or by volume per volume)]. In some embodiments, a concentration of each of one or more surfactants in a lysing composition is in a range of from 0.05% to 2% [e.g., by volume (e.g., by weight per volume or by volume per volume)]. In some embodiments, a concentration of each of one or more surfactants in a lysing composition is in a range of from 0.1% to 2% [e.g., by volume (e.g., by weight per volume or by volume per volume)]. In some embodiments, a concentration of each of one or more surfactants in a lysing composition is in a range of from 0.5% to 2% [e.g., by volume (e.g., by weight per volume or by volume per volume)]. In some embodiments, a concentration of each of one or more surfactants in a lysing composition is in a range of from 0.01% to 1.5% [e.g., by volume (e.g., by weight per volume or by volume per volume)]. In some embodiments, a concentration of each of one or more surfactants in a lysing composition is in a range of from 0.025% to 1.5% [e.g., by volume (e.g., by weight per volume or by volume per volume)]. In some embodiments, a concentration of each of one or more surfactants in a lysing composition is in a range of from 0.05% to 1.5% [e.g., by volume (e.g., by weight per volume or by volume per volume)]. In some embodiments, a concentration of each of one or more surfactants in a lysing composition is in a range of from 0.1% to 1.5% [e.g., by volume (e.g., by weight per volume or by volume per volume)]. In some embodiments, a concentration of each of one or more surfactants in a lysing composition is in a range of from 0.25% to 1.5% [e.g., by volume (e.g., by weight per volume or by volume per volume)]. In some embodiments, a concentration of each of one or more surfactants in a lysing composition is in a range of from 0.5% to 1.5% [e.g., by volume (e.g., by weight per volume or by volume per volume)]. In some embodiments, a concentration of each of one or more surfactants in a lysing composition is in a range of from 0.01% to 1% [e.g., by volume (e.g., by weight per volume or by volume per volume)]. In some embodiments, a concentration of each of one or more surfactants in a lysing composition is in a range of from 0.025% to 1% [e.g., by volume (e.g., by weight per volume or by volume per volume)]. In some embodiments, a concentration of each of one or more surfactants in a lysing composition is in a range of from 0.05% to 1% [e.g., by volume (e.g., by weight per volume or by volume per volume)]. In some embodiments, a concentration of surfactants in a lysing composition is in a range of from 0.1% to 2% [e.g., by volume (e.g., by weight per volume or by volume per volume)]. In some embodiments, a concentration of surfactants in a lysing composition is in a range of from 0.1% to 1.5% [e.g., by volume (e.g., by weight per volume or by volume per volume)]. In some embodiments, a concentration of surfactants in a lysing composition is in a range of from 0.1% to 1% [e.g., by volume (e.g., by weight per volume or by volume per volume)]. In some embodiments, a concentration of surfactants in a lysing composition is in a range of from 0.5% to 1% [e.g., by volume (e.g., by weight per volume or by volume per volume)]. In some embodiments, a concentration of surfactants in a lysing composition is in a range of from 0.1% to 1% [e.g., by volume (e.g., by weight per volume or by volume per volume)]. In some embodiments, a concentration of surfactants in a lysing composition is in a range of from 0.25% to 1% [e.g., by volume (e.g., by weight per volume or by volume per volume)]. In some embodiments, a concentration of surfactants in a lysing composition is in a range of from 0.01% to 1% [e.g., by volume (e.g., by weight per volume or by volume per volume)]. In some embodiments, a concentration of surfactants in a lysing composition is in a range of from 0.025% to 1% [e.g., by volume (e.g., by weight per volume or by volume per volume)]. In some embodiments, a concentration of surfactants in a lysing composition is in a range of from 0.05% to 1% [e.g., by volume (e.g., by weight per volume or by volume per volume)]. In some embodiments, a concentration of surfactants in a lysing composition is in a range of from 0.01% to 2% [e.g., by volume (e.g., by weight per volume or by volume per volume)]. In some embodiments, a concentration of surfactants in a lysing composition is in a range of from 0.025% to 2% [e.g., by volume (e.g., by weight per volume or by volume per volume)]. In some embodiments, a concentration of surfactants in a lysing composition is in a range of from 0.05% to 2% [e.g., by volume (e.g., by weight per volume or by volume per volume)].

Those of skill in the art will appreciate that a surfactant (e.g., detergent) concentration may be measured by volume, where the particular value used is a percentage weight per volume or a percentage volume per volume, depending on the particular surfactant under consideration. For example, concentration of poloxamers is typically measured as weight per volume whereas concentration of Triton X-100 is typically measured as volume per volume.

In some embodiments, a concentration of each of one or more surfactants in a total volume of a lysing composition and a blood sample when the lysing composition and the blood sample are first contacted is no more than 10% [e.g., by volume (e.g., by weight per volume or by volume per volume)]. In some embodiments, a concentration of each of one or more surfactants in a total volume of a lysing composition and a blood sample when the lysing composition and the blood sample are first contacted is no more than 5% [e.g., by volume (e.g., by weight per volume or by volume per volume)]. In some embodiments, a concentration of each of one or more surfactants in a total volume of a lysing composition and a blood sample when the lysing composition and the blood sample are first contacted is no more than 4% [e.g., by volume (e.g., by weight per volume or by volume per volume)]. In some embodiments, a concentration of each of one or more surfactants in a total volume of a lysing composition and a blood sample when the lysing composition and the blood sample are first contacted is no more than 3% [e.g., by volume (e.g., by weight per volume or by volume per volume)]. In some embodiments, a concentration of each of one or more surfactants in a total volume of a lysing composition and a blood sample when the lysing composition and the blood sample are first contacted is no more than 2% [e.g., by volume (e.g., by weight per volume or by volume per volume)]. In some embodiments, a concentration of each of one or more surfactants in a total volume of a lysing composition and a blood sample when the lysing composition and the blood sample are first contacted is no more than 1% [e.g., by volume (e.g., by weight per volume or by volume per volume)]. In some embodiments, a concentration of each of one or more surfactants in a total volume of a lysing composition and a blood sample when the lysing composition and the blood sample are first contacted is no more than 0.5% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.01% to 3% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.025% to 3% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.05% to 3% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.1% to 3% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.5% to 3% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.01% to 2% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.025% to 2% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.05% to 2% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.1% to 2% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.5% to 2% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.01% to 1.5% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.025% to 1.5% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.05% to 1.5% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.1% to 1.5% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.25% to 1.5% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.5% to 1.5% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.01% to 1% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.025% to 1% [e.g., by volume (e.g., by weight per volume or by volume per volume)]. In some embodiments, a concentration of each of one or more surfactants in a total volume of a lysing composition and a blood sample when the lysing composition and the blood sample are first contacted is in a range of from 0.05% to 1% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.1% to 2% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.1% to 1.5% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.1% to 1% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.5% to 1% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.1% to 1% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.25% to 1% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.01% to 1% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.025% to 1% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.05% to 1% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.01% to 2% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.025% to 2% [e.g., by volume (e.g., by weight per volume or by volume per volume)]. In some embodiments, a concentration of surfactants in a total volume of a lysing composition and a blood sample when the lysing composition and the blood sample are first contacted is in a range of from 0.05% to 2% [e.g., by volume (e.g., by weight per volume or by volume per volume)].

In some embodiments, a concentration of a surfactant in a total volume of a lysing composition and a blood sample when the lysing composition and the blood sample are first contacted is in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%). In some embodiments, a concentration of a surfactant in a total volume of a lysing composition and a blood sample when the lysing composition and the blood sample are first contacted is in a range of from 0.01% to 0.3% (e.g., from 0.01% to 0.2%, from 0.01% to 0.1%, from 0.025% to 0.2%, or from 0.025% to 0.1%). In some embodiments, a concentration of a surfactant in a total volume of a lysing composition and a blood sample when the lysing composition and the blood sample are first contacted is in a range of from 0.4% to 5% (e.g., from 0.4% to 2%, from 0.4% to 1%, from 0.4% to 0.8%, or from 0.4% to 0.6%).

In some embodiments, a lysing composition comprises one or more detergents. In some embodiments, a lysing composition comprises one or more non-ionic detergents. In some embodiments, a concentration of each of one or more detergents in a lysing composition is no more than 10% [e.g., by volume (e.g., by weight per volume or by volume per volume)]. In some embodiments, a concentration of each of one or more detergents in a lysing composition is no more than 5% [e.g., by volume (e.g., by weight per volume or by volume per volume)]. In some embodiments, a concentration of each of one or more detergents in a lysing composition is no more than 4% [e.g., by volume (e.g., by weight per volume or by volume per volume)]. In some embodiments, a concentration of each of one or more detergents in a lysing composition is no more than 3% [e.g., by volume (e.g., by weight per volume or by volume per volume)]. In some embodiments, a concentration of each of one or more detergents in a lysing composition is no more than 2% [e.g., by volume (e.g., by weight per volume or by volume per volume)]. In some embodiments, a concentration of each of one or more detergents in a lysing composition is no more than 1% [e.g., by volume (e.g., by weight per volume or by volume per volume)]. In some embodiments, a concentration of each of one or more detergents in a lysing composition is no more than 0.5% [e.g., by volume (e.g., by weight per volume or by volume per volume)]. In some embodiments, a concentration of each of one or more detergents in a lysing composition is in a range of from 0.01% to 3% [e.g., by volume (e.g., by weight per volume or by volume per volume)]. In some embodiments, a concentration of each of one or more detergents in a lysing composition is in a range of from 0.1% to 3% [e.g., by volume (e.g., by weight per volume or by volume per volume)]. In some embodiments, a concentration of each of one or more detergents in a lysing composition is in a range of from 0.5% to 3% [e.g., by volume (e.g., by weight per volume or by volume per volume)]. In some embodiments, a concentration of each of one or more detergents in a lysing composition is in a range of from 0.1% to 2% [e.g., by volume (e.g., by weight per volume or by volume per volume)]. In some embodiments, a concentration of each of one or more detergents in a lysing composition is in a range of from 0.5% to 2% [e.g., by volume (e.g., by weight per volume or by volume per volume)]. In some embodiments, a concentration of each of one or more detergents in a lysing composition is in a range of from 0.1% to 1.5% [e.g., by volume (e.g., by weight per volume or by volume per volume)]. In some embodiments, a concentration of each of one or more detergents in a lysing composition is in a range of from 0.25% to 1.5% [e.g., by volume (e.g., by weight per volume or by volume per volume)]. In some embodiments, a concentration of each of one or more detergents in a lysing composition is in a range of from 0.5% to 1.5% [e.g., by volume (e.g., by weight per volume or by volume per volume)]. In some embodiments, a concentration of detergents in a lysing composition is in a range of from 0.1% to 1% [e.g., by volume (e.g., by weight per volume or by volume per volume)]. In some embodiments, a concentration of detergents in a lysing composition is in a range of from 0.5% to 1% [e.g., by volume (e.g., by weight per volume or by volume per volume)]. In some embodiments, a concentration of detergents in a lysing composition is in a range of from 0.01% to 1% [e.g., by volume (e.g., by weight per volume or by volume per volume)]. In some embodiments, a concentration of detergents in a lysing composition is in a range of from 0.025% to 1% [e.g., by volume (e.g., by weight per volume or by volume per volume)]. In some embodiments, a concentration of detergents in a lysing composition is in a range of from 0.05% to 1% [e.g., by volume (e.g., by weight per volume or by volume per volume)].

In some embodiments, a concentration of each of one or more detergents in a total volume of a lysing composition and a blood sample when the lysing composition and the blood sample are first contacted is no more than 10% [e.g., by volume (e.g., by weight per volume or by volume per volume)]. In some embodiments, a concentration of each of one or more detergents in a total volume of a lysing composition and a blood sample when the lysing composition and the blood sample are first contacted is no more than 5% [e.g., by volume (e.g., by weight per volume or by volume per volume)]. In some embodiments, a concentration of each of one or more detergents in a total volume of a lysing composition and a blood sample when the lysing composition and the blood sample are first contacted is no more than 4% [e.g., by volume (e.g., by weight per volume or by volume per volume)]. In some embodiments, a concentration of each of one or more detergents in a total volume of a lysing composition and a blood sample when the lysing composition and the blood sample are first contacted is no more than 3% [e.g., by volume (e.g., by weight per volume or by volume per volume)]. In some embodiments, a concentration of each of one or more detergents in a total volume of a lysing composition and a blood sample when the lysing composition and the blood sample are first contacted is no more than 2% [e.g., by volume (e.g., by weight per volume or by volume per volume)]. In some embodiments, a concentration of each of one or more detergents in a total volume of a lysing composition and a blood sample when the lysing composition and the blood sample are first contacted is no more than 1% [e.g., by volume (e.g., by weight per volume or by volume per volume)]. In some embodiments, a concentration of each of one or more detergents in a total volume of a lysing composition and a blood sample when the lysing composition and the blood sample are first contacted is no more than 0.5% [e.g., by volume (e.g., by weight per volume or by volume per volume)]. In some embodiments, a concentration of each of one or more detergents in a total volume of a lysing composition and a blood sample when the lysing composition and the blood sample are first contacted is in a range of from 0.01% to 3% [e.g., by volume (e.g., by weight per volume or by volume per volume)]. In some embodiments, a concentration of each of one or more detergents in a total volume of a lysing composition and a blood sample when the lysing composition and the blood sample are first contacted is in a range of from 0.1% to 3% [e.g., by volume (e.g., by weight per volume or by volume per volume)]. In some embodiments, a concentration of each of one or more detergents in a total volume of a lysing composition and a blood sample when the lysing composition and the blood sample are first contacted is in a range of from 0.5% to 3% [e.g., by volume (e.g., by weight per volume or by volume per volume)]. In some embodiments, a concentration of each of one or more detergents in a total volume of a lysing composition and a blood sample when the lysing composition and the blood sample are first contacted is in a range of from 0.1% to 2% [e.g., by volume (e.g., by weight per volume or by volume per volume)]. In some embodiments, a concentration of each of one or more detergents in a total volume of a lysing composition and a blood sample when the lysing composition and the blood sample are first contacted is in a range of from 0.5% to 2% [e.g., by volume (e.g., by weight per volume or by volume per volume)]. In some embodiments, a concentration of each of one or more detergents in a total volume of a lysing composition and a blood sample when the lysing composition and the blood sample are first contacted is in a range of from 0.1% to 1.5% [e.g., by volume (e.g., by weight per volume or by volume per volume)]. In some embodiments, a concentration of each of one or more detergents in a total volume of a lysing composition and a blood sample when the lysing composition and the blood sample are first contacted is in a range of from 0.25% to 1.5% [e.g., by volume (e.g., by weight per volume or by volume per volume)]. In some embodiments, a concentration of each of one or more detergents in a total volume of a lysing composition and a blood sample when the lysing composition and the blood sample are first contacted is in a range of from 0.5% to 1.5% [e.g., by volume (e.g., by weight per volume or by volume per volume)]. In some embodiments, a concentration of detergents in a total volume of a lysing composition and a blood sample when the lysing composition and the blood sample are first contacted is in a range of from 0.1% to 1% [e.g., by volume (e.g., by weight per volume or by volume per volume)]. In some embodiments, a concentration of detergents in a total volume of a lysing composition and a blood sample when the lysing composition and the blood sample are first contacted is in a range of from 0.5% to 1% [e.g., by volume (e.g., by weight per volume or by volume per volume)]. In some embodiments, a concentration of detergents in a total volume of a lysing composition and a blood sample when the lysing composition and the blood sample are first contacted is in a range of from 0.01% to 1% [e.g., by volume (e.g., by weight per volume or by volume per volume)]. In some embodiments, a concentration of detergents in a total volume of a lysing composition and a blood sample when the lysing composition and the blood sample are first contacted is in a range of from 0.025% to 1% [e.g., by volume (e.g., by weight per volume or by volume per volume)]. In some embodiments, a concentration of detergents in a total volume of a lysing composition and a blood sample when the lysing composition and the blood sample are first contacted is in a range of from 0.05% to 1% [e.g., by volume (e.g., by weight per volume or by volume per volume)].

In some embodiments, a lysing composition comprises a surfactant. It has been observed that using saponin as a surfactant in a lysing composition contacted to a blood sample, particularly to fresh whole blood, may be gentler (e.g., less damaging) to an infectious agent (e.g., bacteria or fungus). This may be advantageous, for example, for maintaining infectious agent (e.g., bacteria or fungus) viability in blood samples and recovery of the infectious agent for downstream analysis (e.g., phenotyping) (e.g., using a microfluidics device) and/or further culturing. In comparison, it has been observed that some surfactants, such as Triton X-100 and/or IGEPAL CA-630, while being effective at lysing blood cells, may also result in more infectious agent (e.g., bacterial cell) disruption compared to saponin. Thus, using a combination of multiple surfactants may improve (e.g., maximize) the lysing of RBCs (e.g., the RBC lysis percentage), while maintaining sample viability for further analysis (e.g., phenotyping) of the sample. It may therefore be preferable to include Triton X-100 and/or IGEPAL CA-630 at lower concentration than a saponin. In some embodiments, a surfactant is a saponin. In some embodiments, the saponin concentration in a lysing composition is in a range of from 0.01% to 3% [e.g., by volume (e.g., by weight per volume)]. In some embodiments, the saponin concentration in a lysing composition is in a range of from 0.1% to 3% [e.g., by volume (e.g., by weight per volume)]. In some embodiments, the saponin concentration in a lysing composition is in a range of from 0.5% to 3% [e.g., by volume (e.g., by weight per volume)]. In some embodiments, the saponin concentration in a lysing composition is in a range of from 0.1% to 2% [e.g., by volume (e.g., by weight per volume)]. In some embodiments, the saponin concentration in a lysing composition is in a range of from 0.5% to 2% [e.g., by volume (e.g., by weight per volume)]. In some embodiments, the saponin concentration in a lysing composition is in a range of from 0.1% to 1.5% [e.g., by volume (e.g., by weight per volume)]. In some embodiments, the saponin concentration in a lysing composition is in a range of from 0.25% to 1.5% [e.g., by volume (e.g., by weight per volume)]. In some embodiments, the saponin concentration in a lysing composition is in a range of from 0.5% to 1.5% [e.g., by volume (e.g., by weight per volume)]. In some embodiments, the saponin concentration in a lysing composition is in a range of from 0.1% to 1% [e.g., by volume (e.g., by weight per volume)]. In some embodiments, the saponin concentration in a lysing composition is in a range of from 0.5% to 1% [e.g., by volume (e.g., by weight per volume)]. In some embodiments, a surfactant is Triton X-100. In some embodiments, a concentration of Triton X-100 in a lysing composition is no more than 1% [e.g., by volume (e.g., by volume per volume)]. In some embodiments, a concentration of Triton X-100 in a lysing composition is no more than 0.8% [e.g., by volume (e.g., by volume per volume)]. In some embodiments, a concentration of Triton X-100 in a lysing composition is no more than 0.5% [e.g., by volume (e.g., by volume per volume)]. In some embodiments, a concentration of Triton X-100 in a lysing composition is no more than 0.3% [e.g., by volume (e.g., by volume per volume)]. In some embodiments, a concentration of Triton X-100 in a lysing composition is no more than 0.1% [e.g., by volume (e.g., by volume per volume)].

In some embodiments, the saponin concentration in a total volume of a lysing composition and a blood sample when the lysing composition and the blood sample are first contacted is in a range of from 0.01% to 3% [e.g., by volume (e.g., by weight per volume)]. In some embodiments, the saponin concentration in a total volume of a lysing composition and a blood sample when the lysing composition and the blood sample are first contacted is in a range of from 0.1% to 3% [e.g., by volume (e.g., by weight per volume)]. In some embodiments, the saponin concentration in a total volume of a lysing composition and a blood sample when the lysing composition and the blood sample are first contacted is in a range of from 0.5% to 3% [e.g., by volume (e.g., by weight per volume)]. In some embodiments, the saponin concentration in a total volume of a lysing composition and a blood sample when the lysing composition and the blood sample are first contacted is in a range of from 0.1% to 2% [e.g., by volume (e.g., by weight per volume)]. In some embodiments, the saponin concentration in a total volume of a lysing composition and a blood sample when the lysing composition and the blood sample are first contacted is in a range of from 0.5% to 2% [e.g., by volume (e.g., by weight per volume)]. In some embodiments, the saponin concentration in a total volume of a lysing composition and a blood sample when the lysing composition and the blood sample are first contacted is in a range of from 0.1% to 1.5% [e.g., by volume (e.g., by weight per volume)]. In some embodiments, the saponin concentration in a total volume of a lysing composition and a blood sample when the lysing composition and the blood sample are first contacted is in a range of from 0.25% to 1.5% [e.g., by volume (e.g., by weight per volume)]. In some embodiments, the saponin concentration in a total volume of a lysing composition and a blood sample when the lysing composition and the blood sample are first contacted is in a range of from 0.5% to 1.5% [e.g., by volume (e.g., by weight per volume)]. In some embodiments, the saponin concentration in a total volume of a lysing composition and a blood sample when the lysing composition and the blood sample are first contacted is in a range of from 0.1% to 1% [e.g., by volume (e.g., by weight per volume)]. In some embodiments, the saponin concentration in a total volume of a lysing composition and a blood sample when the lysing composition and the blood sample are first contacted is in a range of from 0.5% to 1% [e.g., by volume (e.g., by weight per volume)]. In some embodiments, a surfactant is Triton X-100. In some embodiments, a concentration of Triton X-100 in a total volume of a lysing composition and a blood sample when the lysing composition and the blood sample are first contacted is no more than 1% [e.g., by volume (e.g., by volume per volume)]. In some embodiments, a concentration of Triton X-100 in a total volume of a lysing composition and a blood sample when the lysing composition and the blood sample are first contacted is no more than 0.8% [e.g., by volume (e.g., by volume per volume)]. In some embodiments, a concentration of Triton X-100 in a total volume of a lysing composition and a blood sample when the lysing composition and the blood sample are first contacted is no more than 0.5% [e.g., by volume (e.g., by volume per volume)]. In some embodiments, a concentration of Triton X-100 in a total volume of a lysing composition and a blood sample when the lysing composition and the blood sample are first contacted is no more than 0.3% [e.g., by volume (e.g., by volume per volume)]. In some embodiments, a concentration of Triton X-100 in a total volume of a lysing composition and a blood sample when the lysing composition and the blood sample are first contacted is no more than 0.1% [e.g., by volume (e.g., by volume per volume)].

In some embodiments, a surfactant in lysing agent is a poloxamer (e.g., Poloxamer 407, Pluronic F-127, Pluronic F-108, Pluronic F-68). Poloxamers are relatively high viscosity polymers, especially compared to other surfactants that may be used in lysing agent. Using a poloxamer as a surfactant in a lysing composition may therefore be used to control viscosity of the lysing composition and/or lysed blood composition resulting from the lysing composition and a blood sample. Other surfactants, or lysing agent components, such as salts, are generally of relatively low viscosity and therefore may not be suitable to control viscosity of a lysing composition and/or lysed blood composition. Viscosity of a lysed blood composition may be desirably controlled, for example, to control (e.g., improve) one or more fluid flow characteristics, such as flow rate, of the lysed blood composition in a microfluidic device for further analysis (e.g., phenotyping). Viscosity may be controlled, for example, to ensure no clogging of in microfluidic device. In some embodiments, a surfactant is Pluronic F-127.

In some embodiments, a concentration of Poloxamer (e.g., Pluronic F-127) in a lysing composition is no more than 2% [e.g., by volume (e.g., by weight per volume)]. In some embodiments, a concentration of Poloxamer (e.g., Pluronic F-127) in a lysing composition is no more than 1% [e.g., by volume (e.g., by weight per volume)]. In some embodiments, a concentration of Poloxamer (e.g., Pluronic F-127) in a lysing composition is no more than 0.8% [e.g., by volume (e.g., by weight per volume)]. In some embodiments, a concentration of Poloxamer (e.g., Pluronic F-127) in a lysing composition is no more than 0.5% [e.g., by volume (e.g., by weight per volume)]. In some embodiments, a concentration of Poloxamer (e.g., Pluronic F-127) in a lysing composition is no more than 0.3% [e.g., by volume (e.g., by weight per volume)]. In some embodiments, a concentration of Poloxamer (e.g., Pluronic F-127) in a lysing composition is no more than 0.1% [e.g., by volume (e.g., by weight per volume)]. In some embodiments, a concentration of Poloxamer (e.g., Pluronic F-127) in a lysing composition is at least 0.1% [e.g., by volume (e.g., by weight per volume)]. In some embodiments, a concentration of Poloxamer (e.g., Pluronic F-127) in a lysing composition is at least 0.25% [e.g., by volume (e.g., by weight per volume)].

In some embodiments, a concentration of Poloxamer (e.g., Pluronic F-127) in a total volume of a lysing composition and a blood sample when the lysing composition and the blood sample are first contacted is no more than 2% [e.g., by volume (e.g., by weight per volume)]. In some embodiments, a concentration of Poloxamer (e.g., Pluronic F-127) in a total volume of a lysing composition and a blood sample when the lysing composition and the blood sample are first contacted is no more than 1% [e.g., by volume (e.g., by weight per volume)]. In some embodiments, a concentration of Poloxamer (e.g., Pluronic F-127) in a total volume of a lysing composition and a blood sample when the lysing composition and the blood sample are first contacted is no more than 0.8% [e.g., by volume (e.g., by weight per volume)]. In some embodiments, a concentration of Poloxamer (e.g., Pluronic F-127) in a total volume of a lysing composition and a blood sample when the lysing composition and the blood sample are first contacted is no more than 0.5% [e.g., by volume (e.g., by weight per volume)]. In some embodiments, a concentration of Poloxamer (e.g., Pluronic F-127) in a total volume of a lysing composition and a blood sample when the lysing composition and the blood sample are first contacted is no more than 0.3% [e.g., by volume (e.g., by weight per volume)]. In some embodiments, a concentration of Poloxamer (e.g., Pluronic F-127) in a total volume of a lysing composition and a blood sample when the lysing composition and the blood sample are first contacted is no more than 0.1% [e.g., by volume (e.g., by weight per volume)]. In some embodiments, a concentration of Poloxamer (e.g., Pluronic F-127) in a total volume of a lysing composition and a blood sample when the lysing composition and the blood sample are first contacted is at least 0.1% [e.g., by volume (e.g., by weight per volume)]. In some embodiments, a concentration of Poloxamer (e.g., Pluronic F-127) in a total volume of a lysing composition and a blood sample when the lysing composition and the blood sample are first contacted is at least 0.25% [e.g., by volume (e.g., by weight per volume)].

In some embodiments, a lysing composition comprises a surfactant that is a detergent. In some embodiments, a lysing composition comprises a detergent. In some embodiments, a lysing composition comprises one or more non-detergent surfactants and one or more detergents. In some embodiments, a detergent is a saponin. In some embodiments, the saponin concentration in a lysing composition is in a range of from 0.01% to 3% [e.g., by volume (e.g., by weight per volume)]. In some embodiments, the saponin concentration in a lysing composition is in a range of from 0.1% to 3% [e.g., by volume (e.g., by weight per volume)]. In some embodiments, the saponin concentration in a lysing composition is in a range of from 0.5% to 3% [e.g., by volume (e.g., by weight per volume)]. In some embodiments, the saponin concentration in a lysing composition is in a range of from 0.1% to 2% [e.g., by volume (e.g., by weight per volume)]. In some embodiments, the saponin concentration in a lysing composition is in a range of from 0.5% to 2% [e.g., by volume (e.g., by weight per volume)]. In some embodiments, the saponin concentration in a lysing composition is in a range of from 0.1% to 1.5% [e.g., by volume (e.g., by weight per volume)]. In some embodiments, the saponin concentration in a lysing composition is in a range of from 0.25% to 1.5% [e.g., by volume (e.g., by weight per volume)]. In some embodiments, the saponin concentration in a lysing composition is in a range of from 0.5% to 1.5% [e.g., by volume (e.g., by weight per volume)]. In some embodiments, the saponin concentration in a lysing composition is in a range of from 0.1% to 1% [e.g., by volume (e.g., by weight per volume)]. In some embodiments, the saponin concentration in a lysing composition is in a range of from 0.5% to 1% [e.g., by volume (e.g., by weight per volume)]. In some embodiments, a detergent is Triton X-100. In some embodiments, a concentration of Triton X-100 in a lysing composition is no more than 1% [e.g., by volume (e.g., by volume per volume)]. In some embodiments, a concentration of Triton X-100 in a lysing composition is no more than 0.8% [e.g., by volume (e.g., by volume per volume)]. In some embodiments, a concentration of Triton X-100 in a lysing composition is no more than 0.5% [e.g., by volume (e.g., by volume per volume)]. In some embodiments, a concentration of Triton X-100 in a lysing composition is no more than 0.3% [e.g., by volume (e.g., by volume per volume)]. In some embodiments, a concentration of Triton X-100 in a lysing composition is no more than 0.1% [e.g., by volume (e.g., by volume per volume)].

In some embodiments, the saponin concentration in a total volume of a lysing composition and a blood sample when the lysing composition and the blood sample are first contacted is in a range of from 0.01% to 3% [e.g., by volume (e.g., by weight per volume)]. In some embodiments, the saponin concentration in a total volume of a lysing composition and a blood sample when the lysing composition and the blood sample are first contacted is in a range of from 0.1% to 3% [e.g., by volume (e.g., by weight per volume)]. In some embodiments, the saponin concentration in a total volume of a lysing composition and a blood sample when the lysing composition and the blood sample are first contacted is in a range of from 0.5% to 3% [e.g., by volume (e.g., by weight per volume)]. In some embodiments, the saponin concentration in a total volume of a lysing composition and a blood sample when the lysing composition and the blood sample are first contacted is in a range of from 0.1% to 2% [e.g., by volume (e.g., by weight per volume)]. In some embodiments, the saponin concentration in a total volume of a lysing composition and a blood sample when the lysing composition and the blood sample are first contacted is in a range of from 0.5% to 2% [e.g., by volume (e.g., by weight per volume)]. In some embodiments, the saponin concentration in a total volume of a lysing composition and a blood sample when the lysing composition and the blood sample are first contacted is in a range of from 0.1% to 1.5% [e.g., by volume (e.g., by weight per volume)]. In some embodiments, the saponin concentration in a total volume of a lysing composition and a blood sample when the lysing composition and the blood sample are first contacted is in a range of from 0.25% to 1.5% [e.g., by volume (e.g., by weight per volume)]. In some embodiments, the saponin concentration in a total volume of a lysing composition and a blood sample when the lysing composition and the blood sample are first contacted is in a range of from 0.5% to 1.5% [e.g., by volume (e.g., by weight per volume)]. In some embodiments, the saponin concentration in a total volume of a lysing composition and a blood sample when the lysing composition and the blood sample are first contacted is in a range of from 0.1% to 1% [e.g., by volume (e.g., by weight per volume)]. In some embodiments, the saponin concentration in a total volume of a lysing composition and a blood sample when the lysing composition and the blood sample are first contacted is in a range of from 0.5% to 1% [e.g., by volume (e.g., by weight per volume)]. In some embodiments, a detergent is Triton X-100. In some embodiments, a concentration of Triton X-100 in a total volume of a lysing composition and a blood sample when the lysing composition and the blood sample are first contacted is no more than 1% [e.g., by volume (e.g., by volume per volume)]. In some embodiments, a concentration of Triton X-100 in a total volume of a lysing composition and a blood sample when the lysing composition and the blood sample are first contacted is no more than 0.8% [e.g., by volume (e.g., by volume per volume)]. In some embodiments, a concentration of Triton X-100 in a total volume of a lysing composition and a blood sample when the lysing composition and the blood sample are first contacted is no more than 0.5% [e.g., by volume (e.g., by volume per volume)]. In some embodiments, a concentration of Triton X-100 in a total volume of a lysing composition and a blood sample when the lysing composition and the blood sample are first contacted is no more than 0.3% [e.g., by volume (e.g., by volume per volume)]. In some embodiments, a concentration of Triton X-100 in a total volume of a lysing composition and a blood sample when the lysing composition and the blood sample are first contacted is no more than 0.1% [e.g., by volume (e.g., by volume per volume)].

In some embodiments, a total concentration of surfactants (e.g., inclusive of any detergents) in a lysing composition is no more than 10% [e.g., by volume (e.g., by weight per volume or by volume per volume)]. In some embodiments, a total concentration of surfactants (e.g., inclusive of any detergents) in a lysing composition is no more than 5% [e.g., by volume (e.g., by weight per volume or by volume per volume)]. In some embodiments, a total concentration of surfactants (e.g., inclusive of any detergents) in a lysing composition is no more than 4% [e.g., by volume (e.g., by weight per volume or by volume per volume)]. In some embodiments, a total concentration of surfactants (e.g., inclusive of any detergents) in a lysing composition is no more than 3% [e.g., by volume (e.g., by weight per volume or by volume per volume)]. In some embodiments, a total concentration of surfactants (e.g., inclusive of any detergents) in a lysing composition is no more than 2% [e.g., by volume (e.g., by weight per volume or by volume per volume)]. In some embodiments, a total concentration of surfactants (e.g., inclusive of any detergents) in a lysing composition is no more than 1% [e.g., by volume (e.g., by weight per volume or by volume per volume)]. In some embodiments, a total concentration of surfactants (e.g., inclusive of any detergents) in a lysing composition is in a range of from 0.1% to 3% [e.g., by volume (e.g., by weight per volume or by volume per volume)]. In some embodiments, a total concentration of surfactants (e.g., inclusive of any detergents) in a lysing composition is in a range of from 0.5% to 3% [e.g., by volume (e.g., by weight per volume or by volume per volume)]. In some embodiments, a total concentration of surfactants (e.g., inclusive of any detergents) in a lysing composition is in a range of from 0.1% to 2% [e.g., by volume (e.g., by weight per volume or by volume per volume)]. In some embodiments, a total concentration of surfactants (e.g., inclusive of any detergents) in a lysing composition is in a range of from 0.5% to 2% [e.g., by volume (e.g., by weight per volume or by volume per volume)]. In some embodiments, a total concentration of surfactants (e.g., inclusive of any detergents) in a lysing composition is in a range of from 0.1% to 1.5% [e.g., by volume (e.g., by weight per volume or by volume per volume)]. In some embodiments, a total concentration of surfactants (e.g., inclusive of any detergents) in a lysing composition is in a range of from 0.25% to 1.5% [e.g., by volume (e.g., by weight per volume or by volume per volume)]. In some embodiments, a total concentration of surfactants (e.g., inclusive of any detergents) in a lysing composition is in a range of from 0.5% to 1.5% [e.g., by volume (e.g., by weight per volume or by volume per volume)]. In some embodiments, a total concentration of surfactants (e.g., inclusive of any detergents) in a lysing composition is in a range of from 0.1% to 1% [e.g., by volume (e.g., by weight per volume or by volume per volume)]. In some embodiments, a total concentration of surfactants (e.g., inclusive of any detergents) in a lysing composition is in a range of from 0.5% to 1% [e.g., by volume (e.g., by weight per volume or by volume per volume)].

In some embodiments, a total concentration of surfactants (e.g., inclusive of any detergents) in a total volume of a lysing composition and a blood sample when the lysing composition and the blood sample are first contacted is no more than 10% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is no more than 5% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is no more than 4% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is no more than 3% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is no more than 2% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is no more than 1% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is in a range of from 0.1% to 3% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is in a range of from 0.5% to 3% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is in a range of from 0.1% to 2% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is in a range of from 0.5% to 2% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is in a range of from 0.1% to 1.5% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is in a range of from 0.25% to 1.5% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is in a range of from 0.5% to 1.5% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is in a range of from 0.1% to 1% [e.g., by volume (e.g., by weight per volume or by volume per volume)]. In some embodiments, a total concentration of surfactants (e.g., inclusive of any detergents) in a total volume of a lysing composition and a blood sample when the lysing composition and the blood sample are first contacted is in a range of from 0.5% to 1% [e.g., by volume (e.g., by weight per volume or by volume per volume)].

In some embodiments, a lysing composition comprises two or more surfactants. In some embodiments, a lysing composition comprises three or more surfactants. In some embodiments, three or more surfactants includes a viscosity-controlling surfactant (e.g., a poloxamer), a saponin, and Triton X-100 or IGEPAL CA-630. In some embodiments, three or more surfactants includes a viscosity-controlling surfactant (e.g., a poloxamer), a saponin, and Triton X-100 or IGEPAL CA-630. In some embodiments, two or more surfactants comprise one or more detergents. In some embodiments, two or more surfactants comprise a detergent. In some embodiments, two or more surfactants comprise a non-detergent surfactant. In some embodiments, two or more surfactants comprise a first surfactant and a second surfactant. In some embodiments, two or more surfactants comprise a first surfactant having a concentration in the lysing composition in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the lysing composition in a range of from 0.4% to 10% [e.g., by volume (e.g., by weight per volume or by volume per volume)] and, optionally, a third surfactant having a concentration in the lysing composition in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%) [e.g., by volume (e.g., by weight per volume or by volume per volume)]. In some embodiments, two or more surfactants comprise a first surfactant having a concentration in the lysing composition in a range of from 0.02% to 0.3% [e.g., by volume (e.g., by weight per volume or by volume per volume)] and a second surfactant having a concentration in the lysing composition in a range of from 0.4% to 10% [e.g., by volume (e.g., by weight per volume or by volume per volume)] and, optionally, a third surfactant having a concentration in the lysing composition in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%) [e.g., by volume (e.g., by weight per volume or by volume per volume)]. In some embodiments, two or more surfactants comprise a first surfactant having a concentration in the lysing composition in a range of from 0.03% to 0.3% [e.g., by volume (e.g., by weight per volume or by volume per volume)] and a second surfactant having a concentration in the lysing composition in a range of from 0.4% to 10% [e.g., by volume (e.g., by weight per volume or by volume per volume)] and, optionally, a third surfactant having a concentration in the lysing composition in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%) [e.g., by volume (e.g., by weight per volume or by volume per volume)]. In some embodiments, two or more surfactants comprise a first surfactant having a concentration in the lysing composition in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the lysing composition in a range of from 0.4% to 5% (e.g., from 0.4% to 2%, from 0.4% to 1%, from 0.4% to 0.8%, or from 0.4% to 0.6%) [e.g., by volume (e.g., by weight per volume or by volume per volume)]. In some embodiments, two or more surfactants comprise a first surfactant having a concentration in the lysing composition in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the lysing composition in a range of from 0.4% to 2% [e.g., by volume (e.g., by weight per volume or by volume per volume)]. In some embodiments, two or more surfactants comprise a first surfactant having a concentration in the lysing composition in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the lysing composition in a range of from 0.4% to 1% [e.g., by volume (e.g., by weight per volume or by volume per volume)] and, optionally, a third surfactant having a concentration in the lysing composition in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%). In some embodiments, two or more surfactants comprise a first surfactant having a concentration in the lysing composition in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the lysing composition in a range of from 0.4% to 0.8% [e.g., by volume (e.g., by weight per volume or by volume per volume)] and, optionally, a third surfactant having a concentration in the lysing composition in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%). In some embodiments, two or more surfactants comprise a first surfactant having a concentration in the lysing composition in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the lysing composition in a range of from 0.4% to 0.6% [e.g., by volume (e.g., by weight per volume or by volume per volume)] and, optionally, a third surfactant having a concentration in the lysing composition in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%). In some embodiments, two or more surfactants comprise a first surfactant having a concentration in a lysing composition in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the lysing composition in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%) [e.g., by volume (e.g., by weight per volume or by volume per volume)] and, optionally, a third surfactant having a concentration in the lysing composition in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%). In some embodiments, the first surfactant is a detergent. In some embodiments, the second surfactant is a detergent. In some embodiments, both the first surfactant and the second surfactant are detergents. In some embodiments, two or more surfactants comprise a first detergent and a second detergent. In some embodiments, the first surfactant is Triton X-100 and the second surfactant is a saponin, and, in some embodiments thereof, where present, the third surfactant is a poloxamer. In some embodiments, the first surfactant is Triton X-100 and the second surfactant is a poloxamer (e.g., Poloxamer 407, Pluronic F-127, Pluronic F-108, Pluronic F-68).

In some embodiments, two or more surfactants comprise a first surfactant having a concentration in a total volume of a lysing composition and a blood sample when the lysing composition and the blood sample are first contacted in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the total volume in a range of from 0.4% to 10% [e.g., by volume (e.g., by weight per volume or by volume per volume)] and, optionally, a third surfactant having a concentration in the total volume in a range of from 0.4% to 10% [e.g., by volume (e.g., by weight per volume or by volume per volume)]. In some embodiments, two or more surfactants comprise a first surfactant having a concentration in a total volume of a lysing composition and a blood sample when the lysing composition and the blood sample are first contacted in a range of from 0.02% to 0.3% [e.g., by volume (e.g., by weight per volume or by volume per volume)] and a second surfactant having a concentration in the total volume in a range of from 0.4% to 10% [e.g., by volume (e.g., by weight per volume or by volume per volume)] and, optionally, a third surfactant having a concentration in the total volume in a range of from 0.4% to 10% [e.g., by volume (e.g., by weight per volume or by volume per volume)]. In some embodiments, two or more surfactants comprise a first surfactant having a concentration in a total volume of a lysing composition and a blood sample when the lysing composition and the blood sample are first contacted in a range of from 0.03% to 0.3% [e.g., by volume (e.g., by weight per volume or by volume per volume)] and a second surfactant having a concentration in the total volume in a range of from 0.4% to 10% [e.g., by volume (e.g., by weight per volume or by volume per volume)] and, optionally, a third surfactant having a concentration in the total volume in a range of from 0.4% to 10% [e.g., by volume (e.g., by weight per volume or by volume per volume)]. In some embodiments, two or more surfactants comprise a first surfactant having a concentration in a total volume of a lysing composition and a blood sample when the lysing composition and the blood sample are first contacted in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the total volume in a range of from 0.4% to 5% (e.g., from 0.4% to 2%, from 0.4% to 1%, from 0.4% to 0.8%, or from 0.4% to 0.6%) [e.g., by volume (e.g., by weight per volume or by volume per volume)]. In some embodiments, two or more surfactants comprise a first surfactant having a concentration in a total volume of a lysing composition and a blood sample when the lysing composition and the blood sample are first contacted in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the total volume in a range of from 0.4% to 2% [e.g., by volume (e.g., by weight per volume or by volume per volume)]. In some embodiments, two or more surfactants comprise a first surfactant having a concentration in a total volume of a lysing composition and a blood sample when the lysing composition and the blood sample are first contacted in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the total volume in a range of from 0.4% to 1% [e.g., by volume (e.g., by weight per volume or by volume per volume)] and, optionally, a third surfactant having a concentration in the total volume in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%). In some embodiments, two or more surfactants comprise a first surfactant having a concentration in a total volume of a lysing composition and a blood sample when the lysing composition and the blood sample are first contacted in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the total volume in a range of from 0.4% to 0.8% [e.g., by volume (e.g., by weight per volume or by volume per volume)] and, optionally, a third surfactant having a concentration in the total volume in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%). In some embodiments, two or more surfactants comprise a first surfactant having a concentration in a total volume of a lysing composition and a blood sample when the lysing composition and the blood sample are first contacted in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the total volume in a range of from 0.4% to 0.6% [e.g., by volume (e.g., by weight per volume or by volume per volume)] and, optionally, a third surfactant having a concentration in the total volume in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%). In some embodiments, two or more surfactants comprise a first surfactant having a concentration in a total volume of a lysing composition and a blood sample when the lysing composition and the blood sample are first contacted in a range of from 0.01% to 0.3% (e.g., from 0.01% to 0.2%, from 0.01% to 0.1%, from 0.025% to 0.2%, or from 0.025% to 0.1%) [e.g., by volume (e.g., by weight per volume or by volume per volume)] and a second surfactant having a concentration in the total volume in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%) [e.g., by volume (e.g., by weight per volume or by volume per volume)] and, optionally, a third surfactant having a concentration in the total volume in a range of from 0.25% to 5% (e.g., from 0.25% to 3%, from 0.25% to 2%, from 0.25% to 1%, from 0.25% to 0.75%, from 0.4% to 2%, from 0.4% to 1.5%, from 0.4% to 1%, from 0.4% to 0.75%). In some embodiments, the first surfactant is Triton X-100 and the second surfactant is a saponin, and, in some embodiments thereof, where present, the third surfactant is a poloxamer. In some embodiments, the first surfactant is Triton X-100 and the second surfactant is a poloxamer (e.g., Poloxamer 407, Pluronic F-127, Pluronic F-108, Pluronic F-68).

In some embodiments, a lysing composition comprises magnesium chloride and/or calcium chloride, for example as a cofactor for one or more enzymes in the lysing composition. In some embodiments, a lysing composition comprises magnesium chloride. In some embodiments, a concentration of magnesium chloride in a total volume of a lysing composition and a blood sample when the lysing composition and the blood sample are first contacted is no more than 1 mM (e.g., from 0.1 mM to 0.8 mM, from 0.3 mM to 0.8 mM, from 0.2 mM to 0.7 mM, from 0.3 mM to 0.7 mM, from 0.1 mM to 0.6 mM, from 0.3 mM to 0.6 mM, or from 0.4 mM to 0.6 mM). In some embodiments, a lysing composition comprises calcium chloride. In some embodiments, a concentration of calcium chloride in a total volume of a lysing composition and a blood sample when the lysing composition and the blood sample are first contacted is no more than 1 mM (e.g., from 0.1 mM to 0.8 mM, from 0.1 mM to 0.7 mM, from 0.1 mM to 0.6 mM, from 0.1 mM to 0.5 mM, from 0.1 mM to 0.4 mM, from 0.1 mM to 0.3 mM, from 0.05 mM to 0.55 mM, from 0.05 mM to 0.25 mM, from 0.15 mM to 0.55 mM, from 0.01 mM to 0.08 mM, from 0.01 mM to 0.07 mM, from 0.01 mM to 0.06 mM, from 0.01 mM to 0.05 mM, from 0.01 mM to 0.04 mM, or from 0.01 mM to 0.03 mM). In some embodiments, a lysing composition comprises magnesium chloride and calcium chloride. In some embodiments, a concentration of magnesium chloride and calcium chloride in a total volume of a lysing composition and a blood sample when the lysing composition and the blood sample are first contacted is no more than 10 mM. In some embodiments, the concentration of magnesium chloride and calcium chloride is no more than 5 mM. In some embodiments, a concentration of magnesium chloride and calcium chloride in a total volume of a lysing composition and a blood sample when the lysing composition and the blood sample are first contacted is no more than 4 mM. In some embodiments, a concentration of magnesium chloride and calcium chloride in a total volume of a lysing composition and a blood sample when the lysing composition and the blood sample are first contacted is no more than 3 mM. In some embodiments, the concentration of magnesium chloride and calcium chloride is no more than 2 mM. In some embodiments, a concentration of magnesium chloride and calcium chloride together in a total volume of a lysing composition and a blood sample when the lysing composition and the blood sample are first contacted is no more than 1.5 mM (e.g., from 0.1 mM to 1.3 mM, from 0.3 mM to 1.3 mM, from 0.5 mM to 1.3 mM, from 0.1 mM to 1 mM, from 0.3 mM to 1 mM, from 0.5 mM to 1 mM, from 0.1 mM to 0.9 mM, from 0.1 mM to 0.8 mM, from 0.3 mM to 0.8 mM, from 0.5 mM to 0.8 mM).

One or more enzymes may be used in a lysis composition and/or during the lysis process to further break down byproducts of the lysis process. One or more enzymes may be used to degrade one or more components of a lysed blood composition, such as, for example, nucleic acids (e.g., DNA, RNA) and/or protein aggregates. In some embodiments, a lysing composition comprises one or more enzymes. In some embodiments, a lysing composition comprises one or more cofactors for one or more enzymes in the lysing composition. In some embodiments, an enzyme is DNase I. In some embodiments, an enzyme is RNase. In some embodiments, an enzyme is Proteinase K. In some embodiments, an enzyme is a lipase. In some embodiments, an enzyme is a phospholipase.

In some embodiments, a concentration of each of one or more enzymes in a lysing composition is no more than 50 U/mL. In some embodiments, a concentration of each of one or more enzymes in a lysing composition is at least 1 U/mL and no more than 30 U/mL. In some embodiments, a concentration of each of one or more enzymes in a lysing composition is at least 1 U/mL and no more than 20 U/mL. In some embodiments, a concentration of each of one or more enzymes in a lysing composition is at least 1 U/mL and no more than 10 U/mL. In some embodiments, a concentration of each of one or more enzymes in a lysing composition is at least 5 U/mL and no more than 30 U/mL. In some embodiments, a concentration of each of one or more enzymes in a lysing composition is at least 5 U/mL and no more than 20 U/mL. In some embodiments, a concentration of each of one or more enzymes in a lysing composition is at least 5 U/mL and no more than 10 U/mL. In some embodiments, a concentration of each of one or more enzymes in a lysing composition is at least 10 U/mL and no more than 20 U/mL. In some embodiments, a concentration of each of one or more enzymes in a lysing composition is at least 20 U/mL and no more than 30 U/mL. In some embodiments, a concentration of each of one or more enzymes in a lysing composition is at least 5 U/mL and no more than 15 U/mL. In some embodiments, a concentration of each of one or more enzymes in a lysing composition is at least 15 U/mL and no more than 25 U/mL.

In some embodiments, a concentration of each of one or more enzymes in a total volume of a lysing composition and a blood sample when the lysing composition and the blood sample are first contacted is no more than 50 U/mL. In some embodiments, a concentration of each of one or more enzymes in a total volume of a lysing composition and a blood sample when the lysing composition and the blood sample are first contacted is at least 1 U/mL and no more than 30 U/mL. In some embodiments, a concentration of each of one or more enzymes in a total volume of a lysing composition and a blood sample when the lysing composition and the blood sample are first contacted is at least 1 U/mL and no more than 20 U/mL. In some embodiments, a concentration of each of one or more enzymes in a total volume of a lysing composition and a blood sample when the lysing composition and the blood sample are first contacted is at least 1 U/mL and no more than 10 U/mL. In some embodiments, a concentration of each of one or more enzymes in a total volume of a lysing composition and a blood sample when the lysing composition and the blood sample are first contacted is at least 5 U/mL and no more than 30 U/mL. In some embodiments, a concentration of each of one or more enzymes in a total volume of a lysing composition and a blood sample when the lysing composition and the blood sample are first contacted is at least 5 U/mL and no more than 20 U/mL. In some embodiments, a concentration of each of one or more enzymes in a total volume of a lysing composition and a blood sample when the lysing composition and the blood sample are first contacted is at least 5 U/mL and no more than 10 U/mL. In some embodiments, a concentration of each of one or more enzymes in a total volume of a lysing composition and a blood sample when the lysing composition and the blood sample are first contacted is at least 10 U/mL and no more than 20 U/mL. In some embodiments, a concentration of each of one or more enzymes in a total volume of a lysing composition and a blood sample when the lysing composition and the blood sample are first contacted is at least 20 U/mL and no more than 30 U/mL. In some embodiments, a concentration of each of one or more enzymes in a total volume of a lysing composition and a blood sample when the lysing composition and the blood sample are first contacted is at least 5 U/mL and no more than 15 U/mL. In some embodiments, a concentration of each of one or more enzymes in a total volume of a lysing composition and a blood sample when the lysing composition and the blood sample are first contacted is at least 15 U/mL and no more than 25 U/mL.

In some embodiments, a lysing composition comprises multiple enzymes (e.g., two or more enzymes). In some embodiments, a total concentration of enzymes in a lysing composition is no more than 50 U/mL. In some embodiments, a total concentration of enzymes in a lysing composition is at least 1 U/mL and no more than 30 U/mL. In some embodiments, a total concentration of enzymes in a lysing composition is at least 1 U/mL and no more than 20 U/mL. In some embodiments, a total concentration of enzymes in a lysing composition is at least 1 U/mL and no more than 10 U/mL. In some embodiments, a total concentration of enzymes in a lysing composition is at least 5 U/mL and no more than 30 U/mL. In some embodiments, a total concentration of enzymes in a lysing composition is at least 5 U/mL and no more than 20 U/mL. In some embodiments, a total concentration of enzymes in a lysing composition is at least 5 U/mL and no more than 10 U/mL. In some embodiments, a total concentration of enzymes in a lysing composition is at least 10 U/mL and no more than 20 U/mL. In some embodiments, a total concentration of enzymes in a lysing composition is at least 20 U/mL and no more than 30 U/mL. In some embodiments, a total concentration of enzymes in a lysing composition is at least 30 U/mL and no more than 40 U/mL. In some embodiments, a total concentration of enzymes in a lysing composition is at least 40 U/mL and no more than 50 U/mL. In some embodiments, a total concentration of enzymes in a lysing composition is at least 5 U/mL and no more than 15 U/mL. In some embodiments, a total concentration of enzymes in a lysing composition is at least 15 U/mL and no more than 25 U/mL. In some embodiments, a total concentration of enzymes in a lysing composition is at least 25 U/mL and no more than 35 U/mL. In some embodiments, a total concentration of enzymes in a lysing composition is at least 35 U/mL and no more than 45 U/mL.

In some embodiments, a total concentration of enzymes in a total volume of a lysing composition and a blood sample when the lysing composition and the blood sample are first contacted is no more than 50 U/mL. In some embodiments, a total concentration of enzymes in a total volume of a lysing composition and a blood sample when the lysing composition and the blood sample are first contacted is at least 1 U/mL and no more than 30 U/mL. In some embodiments, a total concentration of enzymes in a total volume of a lysing composition and a blood sample when the lysing composition and the blood sample are first contacted is at least 1 U/mL and no more than 20 U/mL. In some embodiments, a total concentration of enzymes in a total volume of a lysing composition and a blood sample when the lysing composition and the blood sample are first contacted is at least 1 U/mL and no more than 10 U/mL. In some embodiments, a total concentration of enzymes in a total volume of a lysing composition and a blood sample when the lysing composition and the blood sample are first contacted is at least 5 U/mL and no more than 30 U/mL. In some embodiments, a total concentration of enzymes in a total volume of a lysing composition and a blood sample when the lysing composition and the blood sample are first contacted is at least 5 U/mL and no more than 20 U/mL. In some embodiments, a total concentration of enzymes in a total volume of a lysing composition and a blood sample when the lysing composition and the blood sample are first contacted is at least 5 U/mL and no more than 10 U/mL. In some embodiments, a total concentration of enzymes in a total volume of a lysing composition and a blood sample when the lysing composition and the blood sample are first contacted is at least 10 U/mL and no more than 20 U/mL. In some embodiments, a total concentration of enzymes in a total volume of a lysing composition and a blood sample when the lysing composition and the blood sample are first contacted is at least 20 U/mL and no more than 30 U/mL. In some embodiments, a total concentration of enzymes in a total volume of a lysing composition and a blood sample when the lysing composition and the blood sample are first contacted is at least 30 U/mL and no more than 40 U/mL. In some embodiments, a total concentration of enzymes in a total volume of a lysing composition and a blood sample when the lysing composition and the blood sample are first contacted is at least 40 U/mL and no more than 50 U/mL. In some embodiments, a total concentration of enzymes in a total volume of a lysing composition and a blood sample when the lysing composition and the blood sample are first contacted is at least 5 U/mL and no more than 15 U/mL. In some embodiments, a total concentration of enzymes in a total volume of a lysing composition and a blood sample when the lysing composition and the blood sample are first contacted is at least 15 U/mL and no more than 25 U/mL. In some embodiments, a total concentration of enzymes in a total volume of a lysing composition and a blood sample when the lysing composition and the blood sample are first contacted is at least 25 U/mL and no more than 35 U/mL. In some embodiments, a total concentration of enzymes in a total volume of a lysing composition and a blood sample when the lysing composition and the blood sample are first contacted is at least 35 U/mL and no more than 45 U/mL.

In some embodiments, a lysing composition comprises two or more enzymes. In some embodiments, two or more enzymes comprise a first enzyme and a second enzyme. In some embodiments, the first enzyme is DNase I and the second enzyme is Proteinase K.

In some embodiments, two or more enzymes comprise a first enzyme having a concentration in a lysing composition in a range of from 15 U/mL to 30 U/mL and a second enzyme having a concentration in the lysing composition in a range of from 0.5 U/mL to 10 U/mL. In some embodiments, two or more enzymes comprise a first enzyme having a concentration in a lysing composition in a range of from 15 U/mL to 25 U/mL and a second enzyme having a concentration in the lysing composition in a range of from 0.5 U/mL to 10 U/mL. In some embodiments, two or more enzymes comprise a first enzyme having a concentration in a lysing composition in a range of from 18 U/mL to 25 U/mL and a second enzyme having a concentration in the lysing composition in a range of from 0.5 U/mL to 10 U/mL. In some embodiments, two or more enzymes comprise a first enzyme having a concentration in a lysing composition in a range of from 20 U/mL to 25 U/mL and a second enzyme having a concentration in the lysing composition in a range of from 0.5 U/mL to 10 U/mL. In some embodiments, two or more enzymes comprise a first enzyme having a concentration in a lysing composition in a range of from 18 U/mL to 22 U/mL and a second enzyme having a concentration in the lysing composition in a range of from 0.5 U/mL to 10 U/mL. In some embodiments, two or more enzymes comprise a first enzyme having a concentration in a lysing composition in a range of from 15 U/mL to 30 U/mL and a second enzyme having a concentration in the lysing composition in a range of from 1 U/mL to 10 U/mL. In some embodiments, two or more enzymes comprise a first enzyme having a concentration in a lysing composition in a range of from 15 U/mL to 30 U/mL and a second enzyme having a concentration in the lysing composition in a range of from 5 U/mL to 10 U/mL. In some embodiments, two or more enzymes comprise a first enzyme having a concentration in a lysing composition in a range of from 15 U/mL to 25 U/mL and a second enzyme having a concentration in the lysing composition in a range of from 1 U/mL to 10 U/mL. In some embodiments, two or more enzymes comprise a first enzyme having a concentration in a lysing composition in a range of from 15 U/mL to 25 U/mL and a second enzyme having a concentration in the lysing composition in a range of from 5 U/mL to 10 U/mL. In some embodiments, two or more enzymes comprise a first enzyme having a concentration in a lysing composition in a range of from 18 U/mL to 25 U/mL and a second enzyme having a concentration in the lysing composition in a range of from 1 U/mL to 10 U/mL. In some embodiments, two or more enzymes comprise a first enzyme having a concentration in a lysing composition in a range of from 18 U/mL to 25 U/mL and a second enzyme having a concentration in the lysing composition in a range of from 5 U/mL to 10 U/mL. In some embodiments, two or more enzymes comprise a first enzyme having a concentration in a lysing composition in a range of from 20 U/mL to 25 U/mL and a second enzyme having a concentration in the lysing composition in a range of from 1 U/mL to 10 U/mL. In some embodiments, two or more enzymes comprise a first enzyme having a concentration in a lysing composition in a range of from 20 U/mL to 25 U/mL and a second enzyme having a concentration in the lysing composition in a range of from 5 U/mL to 10 U/mL. In some embodiments, two or more enzymes comprise a first enzyme having a concentration in a lysing composition in a range of from 18 U/mL to 22 U/mL and a second enzyme having a concentration in the lysing composition in a range of from 1 U/mL to 10 U/mL. In some embodiments, two or more enzymes comprise a first enzyme having a concentration in a lysing composition in a range of from 18 U/mL to 22 U/mL and a second enzyme having a concentration in the lysing composition in a range of from 5 U/mL to 10 U/mL.

In some embodiments, two or more enzymes comprise a first enzyme having a concentration in a total volume of a lysing composition and a blood sample when the lysing composition and the blood sample are first contacted in a range of from 15 U/mL to 30 U/mL and a second enzyme having a concentration in the total volume in a range of from 0.5 U/mL to 10 U/mL. In some embodiments, two or more enzymes comprise a first enzyme having a concentration in a total volume of a lysing composition and a blood sample when the lysing composition and the blood sample are first contacted in a range of from 15 U/mL to 25 U/mL and a second enzyme having a concentration in the total volume in a range of from 0.5 U/mL to 10 U/mL. In some embodiments, two or more enzymes comprise a first enzyme having a concentration in a total volume of a lysing composition and a blood sample when the lysing composition and the blood sample are first contacted in a range of from 18 U/mL to 25 U/mL and a second enzyme having a concentration in the total volume in a range of from 0.5 U/mL to 10 U/mL. In some embodiments, two or more enzymes comprise a first enzyme having a concentration in a total volume of a lysing composition and a blood sample when the lysing composition and the blood sample are first contacted in a range of from 20 U/mL to 25 U/mL and a second enzyme having a concentration in the total volume in a range of from 0.5 U/mL to 10 U/mL. In some embodiments, two or more enzymes comprise a first enzyme having a concentration in a total volume of a lysing composition and a blood sample when the lysing composition and the blood sample are first contacted in a range of from 18 U/mL to 22 U/mL and a second enzyme having a concentration in the total volume in a range of from 0.5 U/mL to 10 U/mL. In some embodiments, two or more enzymes comprise a first enzyme having a concentration in a total volume of a lysing composition and a blood sample when the lysing composition and the blood sample are first contacted in a range of from 15 U/mL to 30 U/mL and a second enzyme having a concentration in the total volume in a range of from 1 U/mL to 10 U/mL. In some embodiments, two or more enzymes comprise a first enzyme having a concentration in a total volume of a lysing composition and a blood sample when the lysing composition and the blood sample are first contacted in a range of from 15 U/mL to 30 U/mL and a second enzyme having a concentration in the total volume in a range of from 5 U/mL to 10 U/mL. In some embodiments, two or more enzymes comprise a first enzyme having a concentration in a total volume of a lysing composition and a blood sample when the lysing composition and the blood sample are first contacted in a range of from 15 U/mL to 25 U/mL and a second enzyme having a concentration in the total volume in a range of from 1 U/mL to 10 U/mL. In some embodiments, two or more enzymes comprise a first enzyme having a concentration in a total volume of a lysing composition and a blood sample when the lysing composition and the blood sample are first contacted in a range of from 15 U/mL to 25 U/mL and a second enzyme having a concentration in the total volume in a range of from 5 U/mL to 10 U/mL. In some embodiments, two or more enzymes comprise a first enzyme having a concentration in a total volume of a lysing composition and a blood sample when the lysing composition and the blood sample are first contacted in a range of from 18 U/mL to 25 U/mL and a second enzyme having a concentration in the total volume in a range of from 1 U/mL to 10 U/mL. In some embodiments, two or more enzymes comprise a first enzyme having a concentration in a total volume of a lysing composition and a blood sample when the lysing composition and the blood sample are first contacted in a range of from 18 U/mL to 25 U/mL and a second enzyme having a concentration in the total volume in a range of from 5 U/mL to 10 U/mL. In some embodiments, two or more enzymes comprise a first enzyme having a concentration in a total volume of a lysing composition and a blood sample when the lysing composition and the blood sample are first contacted in a range of from 20 U/mL to 25 U/mL and a second enzyme having a concentration in the total volume in a range of from 1 U/mL to 10 U/mL. In some embodiments, two or more enzymes comprise a first enzyme having a concentration in a total volume of a lysing composition and a blood sample when the lysing composition and the blood sample are first contacted in a range of from 20 U/mL to 25 U/mL and a second enzyme having a concentration in the total volume in a range of from 5 U/mL to 10 U/mL. In some embodiments, two or more enzymes comprise a first enzyme having a concentration in a total volume of a lysing composition and a blood sample when the lysing composition and the blood sample are first contacted in a range of from 18 U/mL to 22 U/mL and a second enzyme having a concentration in the total volume in a range of from 1 U/mL to 10 U/mL. In some embodiments, two or more enzymes comprise a first enzyme having a concentration in a total volume of a lysing composition and a blood sample when the lysing composition and the blood sample are first contacted in a range of from 18 U/mL to 22 U/mL and a second enzyme having a concentration in the total volume in a range of from 5 U/mL to 10 U/mL.

In some embodiments, contacting (e.g., combining) (e.g., mixing) a blood sample to a lysing composition comprises lysing at least 80% (e.g., at least 85%, at least 90%, at least 95%, or at least 99%) of red blood cells in a blood sample. In some embodiments, contacting (e.g., combining) (e.g., mixing) a blood sample to a lysing composition comprises lysing at least 80% (e.g., at least 85%, at least 90%, at least 95%, or at least 99%) of blood cells in the blood sample. In some embodiments, a lysing occurs from a period of time of at least 1 minute and no more than 60 minutes. In some embodiments, a lysing occurs from a period of time of at least 10 minutes and no more than 45 minutes. In some embodiments, a lysing occurs from a period of time of at least 20 minutes and no more than 45 minutes. In some embodiments, a lysing occurs from a period of time of at least 20 minutes and no more than 40 minutes. In some embodiments, a lysing occurs from a period of time of at least 25 minutes and no more than 35 minutes. In some embodiments, a lysing occurs from a period of time of at least 30 minutes and no more than 60 minutes. In some embodiments, a lysing occurs from a period of time of at least 30 minutes and no more than 45 minutes. In some embodiments, a lysing occurs from a period of time of at least 20 minutes and no more than 30 minutes. In some embodiments, a lysing occurs from a period of time of at least 10 minutes and no more than 30 minutes. In some embodiments, a lysing occurs from a period of time of at least 1 minute and no more than 10 minutes. In some embodiments, a lysing occurs from a period of time of at least 3 minutes and no more than 7 minutes. In some embodiments, a lysing occurs from a period of time of 30 minutes. In some embodiments, a lysing occurs within a period of time of 10 minutes. In some embodiments, a lysing occurs within a period of time of 5 minutes. While generally shorter lysing times are preferred, and lysing compositions disclosed herein may be used to lyse blood cells (while preserving and/or maintaining viability of one or more infectious agents) in such short time periods, it will be recognized that longer lysing times may be used for larger volumes to achieve similar, same, or higher throughput in terms of amount of sample lysed per unit time.

In some embodiments, a blood sample is a whole blood sample. In some embodiments, a blood sample is a fresh blood sample. In some embodiments, a blood sample is a refrigerated blood sample, for example a refrigerated whole blood sample. In some embodiments, a blood sample is a room temperature blood sample, for example a refrigerated whole blood sample. In some embodiments, a blood sample is a stored blood sample, for example a stored whole blood sample. In some embodiments, a blood sample is from a walking blood bank. In some embodiments, (e.g., prior to a contacting) a blood sample has been frozen. In some embodiments, (e.g., before a contacting) a blood sample has been treated with an anticoagulant (e.g., ethylenediaminetetraacetic acid (EDTA), sodium polyanethole sulfonate (SPS), sodium citrate, lithium heparin) [e.g., wherein a method comprises treating a blood sample with an anticoagulant (e.g., ethylenediaminetetraacetic acid (EDTA), sodium polyanethole sulfonate (SPS), sodium citrate, lithium heparin) prior to a contacting]. In some embodiments, (e.g., before a contacting) a fresh blood sample has been treated with an anticoagulant (e.g., ethylenediaminetetraacetic acid (EDTA), sodium polyanethole sulfonate (SPS), sodium citrate, lithium heparin). In some embodiments, (e.g., before a contacting) a whole blood sample has been treated with an anticoagulant (e.g., ethylenediaminetetraacetic acid (EDTA), sodium polyanethole sulfonate (SPS), sodium citrate, lithium heparin).

In some embodiments, contacting a blood sample to a lysing composition comprises rocking a sample and a lysing composition together. In some embodiments, a rocking occurs from a period of time of at least 1 minute and no more than 60 minutes. In some embodiments, a rocking occurs from a period of time of at least 10 minutes and no more than 45 minutes. In some embodiments, a rocking occurs from a period of time of at least 20 minutes and no more than 45 minutes. In some embodiments, a rocking occurs from a period of time of at least 20 minutes and no more than 40 minutes. In some embodiments, a rocking occurs from a period of time of at least 25 minutes and no more than 35 minutes. In some embodiments, a rocking occurs from a period of time of at least 30 minutes and no more than 60 minutes. In some embodiments, a rocking occurs from a period of time of at least 30 minutes and no more than 45 minutes. In some embodiments, a rocking occurs from a period of time of at least 20 minutes and no more than 30 minutes. In some embodiments, a rocking occurs from a period of time of at least 10 minutes and no more than 30 minutes. In some embodiments, a rocking occurs from a period of time of 30 minutes. In some embodiments, a rocking occurs from a period of time of at least 1 minute and no more than 10 minutes. In some embodiments, a rocking occurs from a period of time of at least 3 minutes and no more than 7 minutes.

In some embodiments, a method comprises lysing blood cells in a blood sample that has been contacted with a lysing composition at an elevated temperature (e.g., above ambient or room temperature). In some embodiments, a method comprises lysing blood cells in a blood sample that has been contacted with a lysing composition at a temperature from at least 20° C. to no more than 50° C. (e.g., to no more than 40° C.). In some embodiments, a method comprises lysing blood cells in a blood sample that has been contacted with a lysing composition at a temperature from at least 25° C. to no more than 50° C. (e.g., to no more than 40° C.). In some embodiments, a method comprises lysing blood cells in a blood sample that has been contacted with a lysing composition at a temperature from at least 25° C. to no more than 37° C. In some embodiments, contacting a blood sample to a lysing composition comprises rocking a sample and a lysing composition together at an elevated temperature (e.g., above ambient or room temperature). In some embodiments, contacting a blood sample to a lysing composition comprises rocking a sample and a lysing composition together at a temperature from at least 20° C. to no more than 50° C. (e.g., to no more than 40° C.). In some embodiments, contacting a blood sample to a lysing composition comprises rocking a sample and a lysing composition together at a temperature from at least 25° C. to no more than 50° C. (e.g., to no more than 40° C.). In some embodiments, contacting a blood sample to a lysing composition comprises rocking a sample and a lysing composition together at a temperature from at least 25° C. to no more than 37° C.

In some embodiments, contacting a blood sample to a lysing composition comprises vortexing a sample and a lysing composition together. In some embodiments, vortexing occurs from a period of time of at least 10 seconds and no more than 1 minute. In some embodiments, vortexing occurs from a period of time of at least 20 seconds and no more than 40 seconds.

In some embodiments, a lysing composition is a portion of a stock solution. In some embodiments, a lysed blood composition is a solution. In some embodiments, a lysed blood composition is substantially devoid of red blood cells (e.g., such that culturing and/or identification and/or AST can be performed using a microfluidics device).

In some embodiments, a method includes performing a centrifugation of a blood sample prior to a contacting. In some embodiments, a blood sample has been centrifuged. In some embodiments, centrifugation occurs at a speed in a range of from 1,000 to 10,000 rotations per minute (rpm). In some embodiments, a speed is in a range of from 2,000 to 8,000 rpm. In some embodiments, a speed is in a range of from 3,000 to 5,000 rpm. In some embodiments, a speed is in a range of from 3,500 to 4,500 rpm. In some embodiments, centrifugation occurs for a period of time in a range of from 1 to 30 minutes. In some embodiments, centrifugation occurs for a period of time in a range of from 5 to 20 minutes. In some embodiments, centrifugation occurs for a period of time in a range of from 10 to 15 minutes. In some preferred embodiments, no centrifugation is used.

In some embodiments, blood sample comprising a bacteria is a supernatant of a centrifugation. In some embodiments, a method includes performing a centrifugation. In some embodiments, centrifugation occurred (e.g., occurs) at a speed in a range of from 10 to 2,500 rotations per minute (rpm). In some embodiments, a speed is in a range of from 100 to 1,500 rpm. In some embodiments, a speed is in a range of from 250 to 1,000 rpm. In some embodiments, a speed is in a range of from 300 to 700 rpm. In some embodiments, a speed is in a range of from 400 to 600 rpm (e.g., at 500 rpm). In some embodiments, a centrifugation occurred (e.g., occurs) for a period of time in a range of from 1 to 30 minutes. In some embodiments, a period of time is in a range of from 5 to 20 minutes. In some embodiments, a period of time is in a range of from 10 to 15 minutes.

In some embodiments, a step of contacting a blood sample and a lysing composition comprises combining a blood sample and a lysing composition in a ratio in a range of from 3:1 to 30:1 (e.g., 4:1 or 24:1). In some embodiments, a ratio is in a range of from 3:1 to 5:1. In some embodiments, a ratio is in a range of from 22:1 to 26:1. In some embodiments, a step of contacting a blood sample and a lysing composition comprises combining a blood sample and a lysing composition such that a blood sample is at least 75% (e.g., at least 80%, at least 85%, at least 90%, or at least 95%) of total volume thereof. In some embodiments, a step of contacting a blood sample and a lysing composition comprises combining a blood sample and a lysing composition in a ratio such that lysing agent from a lysing composition is present in a concentration of at least 0.4% and no more than 3% (e.g., by volume). In some embodiments, a step of contacting a blood sample and a lysing composition comprises combining a blood sample and a lysing composition in a ratio such that lysing agent from a lysing composition is present in a concentration of at least 0.5% and no more than 1.5% (e.g., by volume). In some embodiments, a step of contacting a blood sample and a lysing composition comprises combining a blood sample and a lysing composition in a ratio such that lysing agent from a lysing composition is present in a concentration of at least 0.75% and no more than 1.25% (e.g., by volume). In some embodiments, a step of contacting a blood sample and a lysing composition comprises combining a blood sample and a lysing composition such that a lysing composition is no more than 25% (e.g., no more than 20%, no more than 15%, no more than 10%, or no more than 5%) of total volume thereof.

Combinations (e.g., mixtures) of blood samples and lysing compositions may be at high lysing composition to blood sample ratios instead. Using a high ratio of lysing composition to blood sample may offer several advantages. First, this may enable effective lysis even when a lower volume of blood is available, which may be crucial when sample is limited. Second, this approach may facilitate processing a higher blood volume per unit time, which may improve the speed of diagnostics. In some embodiments, a step of contacting a blood sample and a lysing composition comprises combining a blood sample and a lysing composition in a ratio of blood sample to lysing composition in a range of from 1:30 to 1:1 (e.g., 1:3, 1:5, 1:10, 1:20). In some embodiments, a ratio of blood sample to lysing composition is in a range of from 1:20 to 1:2. In some embodiments, a ratio of blood sample to lysing composition is in a range of from 1:15 to 1:2. In some embodiments, a ratio of blood sample to lysing composition is in a range of from 1:10 to 1:2. In some embodiments, a ratio of blood sample to lysing composition is in a range of from 1:10 to 1:3. In some embodiments, a ratio of blood sample to lysing composition is in a range of from 1:5 to 1:2.

In some embodiments, a lysed blood composition has been formed from a combination of a blood sample and a lysing composition in a ratio of blood sample to lysing composition in a range of from 1:30 to 1:1 (e.g., 1:3, 1:5, 1:10, 1:20), for example a ratio that is in a range of from 1:20 to 1:1 (e.g., from 1:20 to 1:2, or from 1:20 to 1:3), in a range of from 1:15 to 1:1 (e.g., from 1:15 to 1:2, or from 1:15 to 1:3), or in a range of from 1:10 to 1:1 (e.g., from 1:10 to 1:2, or from 1:10 to 1:3). In some embodiments, a lysed blood composition has been formed from a combination of a blood sample and a lysing composition that is in a ratio in a range of from 3:1 to 1:30 (e.g., from 3:1 to 1:10, from 3:1 to 1:5, from 3:1 to 1:3, from 1:1 to 1:10, from 1:1 to 1:5, from 1:1 to 1:3).

In some embodiments, a step of contacting a blood sample and a lysing composition comprises combining a blood sample and a lysing composition such that a blood sample is at least 75% (e.g., at least 80%, at least 85%, at least 90%, or at least 95%) of total volume thereof. In some embodiments, a step of contacting a blood sample and a lysing composition comprises combining a blood sample and a lysing composition in a ratio such that lysing agent from a lysing composition is present in a concentration of at least 0.4% and no more than 3% (e.g., by volume). In some embodiments, a step of contacting a blood sample and a lysing composition comprises combining a blood sample and a lysing composition in a ratio such that lysing agent from a lysing composition is present in a concentration of at least 0.5% and no more than 1.5% (e.g., by volume). In some embodiments, a step of contacting a blood sample and a lysing composition comprises combining a blood sample and a lysing composition in a ratio such that lysing agent from a lysing composition is present in a concentration of at least 0.75% and no more than 1.25% (e.g., by volume). In some embodiments, a step of contacting a blood sample and a lysing composition comprises combining a blood sample and a lysing composition such that a lysing composition is no more than 25% (e.g., no more than 20%, no more than 15%, no more than 10%, or no more than 5%) of total volume thereof.

In some embodiments, a method includes, before contacting, processing a blood sample to reduce its volume by at least 25% (e.g., at least 30%, at least 40%, or at least 50%).

In some embodiments, a method includes culturing a bacteria from a lysed blood composition [e.g., within 8 hours (e.g., within 7 hours, within 6 hours, within 5 hours, within 4 hours, within 3 hours, within 2 hours, within 60 min, within 30 min, within 15 min, or within 10 min) of a contacting]. In some embodiments, a culturing is performed using a microfluidics device. In some embodiments, a microfluidics device comprises single-cell-sized growth trenches and a culturing is performed using a trenches. In some embodiments, a microfluidics device comprises larger than single-cell-sized growth trenches (e.g., multiple-cell-sized growth trenches) and a culturing is performed using the trenches.

In some embodiments, a method includes applying at least a portion of a lysed blood composition to a microfluidics device (e.g., injecting a at least a portion of a lysed blood composition onto or into a microfluidics device). In some embodiments, a method includes identifying an identity (e.g., genus) (e.g., strain) (e.g., type) of an infectious agent using a lysed blood composition (e.g., using a microfluidics device) (e.g., using microscopy) (e.g., using quantitative phase microscopy (quantitative phase-contrast microscopy) and/or hyperspectral imaging). In some embodiments, a method includes identifying an identity (e.g., genus) (e.g., strain) (e.g., type) of a bacteria using a lysed blood composition (e.g., using a microfluidics device) (e.g., using microscopy) (e.g., using quantitative phase microscopy (quantitative phase-contrast microscopy) and/or hyperspectral imaging). In some embodiments, a method includes identifying an identity (e.g., genus) (e.g., strain) (e.g., type) of a fungus using a lysed blood composition (e.g., using a microfluidics device) (e.g., using microscopy) (e.g., using quantitative phase microscopy (quantitative phase-contrast microscopy) and/or hyperspectral imaging). In some embodiments, a method includes performing an antimicrobial susceptibility test (AST) on an infectious agent using a lysed blood composition (e.g., using a microfluidics device). In some embodiments, a method includes performing an antimicrobial susceptibility test (AST) on a bacteria using a lysed blood composition (e.g., using a microfluidics device). In some embodiments, a method includes performing an antimicrobial susceptibility test (AST) on a fungus using a lysed blood composition (e.g., using a microfluidics device).

One or more of various characterization steps may be performed as part of a method disclosed herein using a lysed blood composition obtained as part of the method. Characterization may be characterization of cells (e.g., one or more bacterial cells and/or one or more fungal cells). Characterization may include phenotyping (e.g., using an imaging-based method). Characterization may be performed using one or more imaging-based methods (e.g., technique(s)). Characterization made by one or more steps of a method may include detection (e.g., presence), morphological characterization, size characterization, growth characterization (e.g., growth pattern characterization), Gram classification, or a combination thereof, for example of one or more cells of an infectious agent (e.g., one or more bacterial cells and/or one or more fungal cells). Characterization may include phenotyping (e.g., using an imaging-based method). A method may include detecting one or more infectious agents in a lysed blood composition (e.g., determining presence thereof in the lysed blood composition), determining one or more morphological characteristics (e.g., cell and/or colony shape, morphology, texture, form, elevation and/or depression, or margin) of an infectious agent (e.g., one or more cells thereof) in a lysed blood composition, determining one or more size characteristics [e.g., a point estimate (e.g., an average, mean, median, and/or mode) of characteristic length (e.g., diameter), area, and/or volume] of an infectious agent (e.g., one or more cells thereof) in a lysed blood composition, determining one or more growth characteristics (e.g., one or more growth pattern characteristics) (e.g., growth curve, bacteria division, or propensity for clustering) of an infectious agent (e.g., one or more cells thereof) in a lysed blood composition, classifying Gram class of an infectious agent (e.g., one or more cells thereof) in a lysed blood composition, or a combination thereof. In some embodiments, a method includes obtaining a lysed blood composition and detecting one or more infectious agents in the lysed blood composition (e.g., determining presence thereof in the lysed blood composition). In some embodiments, a method includes obtaining a lysed blood composition and determining one or more morphological characteristics of an infectious agent (e.g., one or more cells thereof) in the lysed blood composition. In some embodiments, a method includes obtaining a lysed blood composition and determining one or more size characteristics [e.g., a point estimate (e.g., an average, mean, median, and/or mode) of characteristic length (e.g., diameter), area, and/or volume] of an infectious agent (e.g., one or more cells thereof) in the lysed blood composition. In some embodiments, a method includes obtaining a lysed blood composition and determining one or more growth characteristics (e.g., one or more growth pattern characteristics) of an infectious agent (e.g., one or more cells thereof) in the lysed blood composition. In some embodiments, a method includes obtaining a lysed blood composition and classifying Gram class of an infectious agent (e.g., one or more cells thereof) in the lysed blood composition. In some embodiments, a method includes obtaining a lysed blood composition and identifying an identity (e.g., genus) (e.g., strain) (e.g., type) of an infectious agent (e.g., one or more cells thereof) in the lysed blood composition. Characterization may occur using (e.g., on) a microfluidics device. One or more characteristics of an infectious agent (e.g., one or more cells thereof) may be characterized using (e.g., on) a microfluidics device.

In some embodiments, identifying an identity (e.g., genus) (e.g., strain) (e.g., type) of an infectious agent (e.g., one or more cells thereof) using a lysed blood composition comprises detecting a presence of the infectious agent (e.g., using a microfluidics device) (e.g., using microscopy) (e.g., using quantitative phase microscopy (quantitative phase-contrast microscopy) and/or hyperspectral imaging). In some embodiments, identifying an identity (e.g., genus) (e.g., strain) (e.g., type) of an infectious agent (e.g., one or more cells thereof) using a lysed blood composition comprises identifying a morphology (e.g., cell and/or colony shape, morphology, texture, form, elevation and/or depression, or margin) of the infectious agent (e.g., using a microfluidics device) (e.g., using microscopy) (e.g., using quantitative phase microscopy (quantitative phase-contrast microscopy) and/or hyperspectral imaging). In some embodiments, identifying an identity (e.g., genus) (e.g., strain) (e.g., type) of an infectious agent (e.g., one or more cells thereof) using a lysed blood composition comprises identifying a size (e.g., cell size or colony size) of the infectious agent (e.g., using a microfluidics device) (e.g., using microscopy) (e.g., using quantitative phase microscopy (quantitative phase-contrast microscopy) and/or hyperspectral imaging). In some embodiments, identifying an identity (e.g., genus) (e.g., strain) (e.g., type) of an infectious agent (e.g., one or more cells thereof) using a lysed blood composition comprises identifying a growth pattern (e.g., growth curve, bacteria division, or propensity for clustering) of the infectious agent (e.g., using flow cytometry) (e.g., using a microfluidics device) (e.g., using microscopy) (e.g., using quantitative phase microscopy (quantitative phase-contrast microscopy) and/or hyperspectral imaging). In some embodiments, identifying an identity (e.g., genus) (e.g., strain) (e.g., type) of infectious agent using a lysed blood composition comprises identifying a Gram classification (e.g., Gram-positive or Gram-negative) of the infectious agent (e.g., using Gram staining) (e.g., using a microfluidics device) (e.g., using microscopy) (e.g., using quantitative phase microscopy (quantitative phase-contrast microscopy) and/or hyperspectral imaging).

In some embodiments, a method includes, after a contacting, quenching a sample with a lysis-ceasing composition. In some embodiments, a lysis-ceasing composition is phosphate buffered saline.

In some embodiments, a method is performed in an assisted living facility (e.g., in a nursing home, a long-term assisted living facility). In some embodiments, a method is performed in a clinic (e.g., a family doctor office, emergency department (ED), intensive care unit (ICU), critical care unit (CCU), short-term acute care hospital, blood donation center, dialysis center, surgery suite, point-of-care clinic, outpatient clinic, veterinary clinic, retail pharmacy, school nurse's office). In some embodiments, a method is performed in a battle field setting (e.g., by a medic) (e.g., in a medic's tent or field hospital).

In some embodiments, stock lysing composition (e.g., stock solution) for lysing a blood sample without destroying bacteria comprised therein (e.g., for lysing a blood sample while maintaining viability of a bacteria) is a solution comprising lysing agent in a concentration of at least 10% (e.g., at least 15%, at least 20%, or at least 25%) (e.g., by volume). Such stock lysing composition may be prepared at relative concentrations of components thereof such that when contacted with a blood sample thereby combining the two (e.g., by passive or active mixing) (e.g., by rocking), the resulting concentration of components of the lysing composition result in a total volume is at desired amount(s) or in desired range(s). In some embodiments, stock lysing composition (e.g., stock solution) comprises lysing agent in a concentration of no more than 10% (e.g., by volume). In some embodiments, stock lysing composition (e.g., stock solution) comprises lysing agent in a concentration of no more than 5% (e.g., by volume). In some embodiments, stock lysing composition (e.g., stock solution) comprises lysing agent in a concentration of no more than 4% (e.g., by volume). In some embodiments, stock lysing composition (e.g., stock solution) comprises lysing agent in a concentration of no more than 3% (e.g., by volume). In some embodiments, stock lysing composition (e.g., stock solution) comprises lysing agent in a concentration of no more than 2% (e.g., by volume). In some embodiments, stock lysing composition (e.g., stock solution) comprises lysing agent in a concentration of no more than 1% (e.g., by volume). In some embodiments, stock lysing composition (e.g., stock solution) comprises lysing agent in a concentration in a range of from 0.1% to 3% (e.g., by volume). In some embodiments, stock lysing composition (e.g., stock solution) comprises lysing agent in a concentration in a range of from 0.5% to 3% (e.g., by volume). In some embodiments, stock lysing composition (e.g., stock solution) comprises lysing agent in a concentration in a range of from 0.1% to 2% (e.g., by volume). In some embodiments, stock lysing composition (e.g., stock solution) comprises lysing agent in a concentration in a range of from 0.5% to 2% (e.g., by volume). In some embodiments, stock lysing composition (e.g., stock solution) comprises lysing agent in a concentration in a range of from 0.1% to 1.5% (e.g., by volume). In some embodiments, stock lysing composition (e.g., stock solution) comprises lysing agent in a concentration in a range of from 0.25% to 1.5% (e.g., by volume). In some embodiments, stock lysing composition (e.g., stock solution) comprises lysing agent in a concentration in a range of from 0.5% to 1.5% (e.g., by volume). In some embodiments, stock lysing composition (e.g., stock solution) comprises lysing agent in a concentration in a range of from 0.1% to 1% (e.g., by volume). In some embodiments, stock lysing composition (e.g., stock solution) comprises lysing agent in a concentration in a range of from 0.5% to 1% (e.g., by volume). In some embodiments, a composition is a lysis buffer composition.

In some embodiments, an infectious agent is a pathogen. In some embodiments, an infectious agent is a bacteria. In some embodiments, a bacteria is a strain of ESKAPE-E bacteria (e.g., Enterococcus faecalis, Staphylococcus aureus, Klebsiella quasipneumoniae, Acinetobacter baumannii, Pseudomonas aeruginosa, Escherichia coli, or Enterobacter spp.). In some embodiments, a bacteria is an ESKAPE-E bacteria (e.g., Enterococcus faecalis, Staphylococcus aureus, Klebsiella quasipneumoniae, Acinetobacter baumannii, Pseudomonas aeruginosa, Escherichia coli, or Enterobacter spp.). In some embodiments, an infectious agent is an ESKAPE-E bacteria (e.g., Enterococcus faecalis, Staphylococcus aureus, Klebsiella quasipneumoniae, Acinetobacter baumannii, Pseudomonas aeruginosa, Escherichia coli, or Enterobacter spp.). In some embodiments, a bacteria comprises a strain of Escherichia coli. In some embodiments, bacteria are gram-negative. In some embodiments, bacteria are gram-positive. In some embodiments, an infectious agent is a fungus. In some embodiments, a fungus is yeast (e.g., Candida spp.). In some embodiments, a fungus is a strain of Candida spp. (e.g., Candida albicans, Candida auris, Candida glabrata, Candida krusei, Candida parapsilosis, or Candida tropicalis). In some embodiments, a fungus is a Candida spp. fungus (e.g., Candida albicans, Candida auris, Candida glabrata, Candida krusei, Candida parapsilosis, or Candida tropicalis). In some embodiments, an infectious agent is a Candida spp. fungus (e.g., Candida albicans, Candida auris, Candida glabrata, Candida krusei, Candida parapsilosis, or Candida tropicalis). In some embodiments, an infectious agent comprises one or more prokaryotic cells. In some embodiments, an infectious agent comprises one or more eukaryotic cells. In some embodiments, an infectious agent is a virus (e.g., bound to particles). A bacteria in a blood sample, or lysed blood composition, may comprise one or more bacteria cells (e.g., of a single strain or multiple strains). A fungus in a blood sample, or lysed blood composition, may comprise one or more fungal cells (e.g., of a single strain or multiple strains).

Lysing compositions disclosed herein (e.g., and evaluated) may maintain infectious agent (e.g., a bacteria or fungus) viability in blood samples (e.g., whole blood samples), for example such that the infectious agent (e.g., the bacteria or fungus) can subsequently be phenotyped (e.g., using an imaging-based method, for example a label-free microscopy technique) and/or cultured (e.g., using a microfluidics device). Lysing compositions disclosed herein (e.g., and evaluated) may keep one or more infectious agents in blood samples (e.g., whole blood samples) intact, for example such that the infectious agent (e.g., the bacteria or fungus) can subsequently be phenotyped (e.g., using an imaging-based technique) and/or cultured (e.g., using a microfluidics device). Infectious agent(s) in blood samples (e.g., whole blood samples) contacted with lysing compositions disclosed herein (e.g., and evaluated) may survive lysing of blood cells (e.g., RBCs). Lysing compositions disclosed herein (e.g., and evaluated) may minimally impact growth curve of cells of an infectious agent (e.g., one or more cells thereof) (e.g., bacteria cells or fungus cells), for example such lysing does not delay assay (e.g., bacteria assay or fungus assay) turnaround time. Methods disclosed herein may prepare a blood sample comprising a bacteria, e.g. for culturing of the bacteria, without delaying bacteria assay turnaround time. Methods disclosed herein may prepare a blood sample comprising fungus, e.g. for culturing of the fungus, without delaying bacteria assay turnaround time.

In some embodiments, a bacteria in a lysed blood composition is viable. In some embodiments, a bacteria in a lysed blood composition is intact. In some embodiments, a bacteria in a lysed blood composition have a growth curve that deviates no more than 20% (e.g., no more than 10%) from a reference growth curve. In some embodiments, a fungus in a lysed blood composition has a growth curve that deviates no more than 20% (e.g., no more than 10%) from a reference growth curve. In some embodiments, a fungus in a lysed blood composition is viable. In some embodiments, a fungus in a lysed blood composition is intact. In some embodiments, a fungus in a lysed blood composition has a growth curve that deviates no more than 20% (e.g., no more than 10%) from a reference growth curve. In some embodiments, an infectious agent in a lysed blood composition is viable. In some embodiments, an infectious agent in a lysed blood composition is intact. In some embodiments, intact infectious agent, for example an intact bacteria or fungus, may be phenotyped (e.g., using an image-based technique) after lysing. In some embodiments, intact infectious agent, for example a preserved bacteria or fungus, may be cultured after lysing. In some embodiments, viable infectious agent, for example an intact bacteria or fungus, may be phenotyped (e.g., using an image-based technique) after lysing. In some embodiments, viable infectious agent, for example a preserved bacteria or fungus, may be cultured after lysing.

Lysis compositions provided herein may be sufficiently gentle to lyse blood cells (e.g., in amounts described elsewhere) while preserving infectious agent (e.g., instead of damaging, degrading, and/or lysing the infectious agent) such that a significant percentage of the infectious agent is preserved after lysing. In some embodiments, a method comprises contacting a blood sample comprising an infectious agent (e.g., a bacteria or fungus) to a lysing composition, wherein at least 50% of the infectious agent is preserved after lysing the blood sample with the lysis composition. In some embodiments, a method comprises contacting a blood sample comprising an infectious agent (e.g., a bacteria or fungus) to a lysing composition, wherein at least 70% of the infectious agent is preserved after lysing the blood sample with the lysis composition. In some embodiments, a method comprises contacting a blood sample comprising an infectious agent (e.g., a bacteria or fungus) to a lysing composition, wherein at least 80% of the infectious agent is preserved after lysing the blood sample with the lysis composition. In some embodiments, a method comprises contacting a blood sample comprising an infectious agent (e.g., a bacteria or fungus) to a lysing composition, wherein at least 85% of the infectious agent is preserved after lysing the blood sample with the lysis composition. In some embodiments, a method comprises contacting a blood sample comprising an infectious agent (e.g., a bacteria or fungus) to a lysing composition, wherein at least 90% of the infectious agent is preserved after lysing the blood sample with the lysis composition. In some embodiments, a method comprises contacting a blood sample comprising an infectious agent (e.g., a bacteria or fungus) to a lysing composition, wherein at least 95% of the infectious agent is preserved after lysing the blood sample with the lysis composition. In some embodiments, a method comprises contacting a blood sample comprising an infectious agent (e.g., a bacteria or fungus) to a lysing composition, wherein at least 98% of the infectious agent is preserved after lysing the blood sample with the lysis composition. In some embodiments, a method comprises contacting a blood sample comprising an infectious agent (e.g., a bacteria or fungus) to a lysing composition, wherein at least 99% of the infectious agent is preserved after lysing the blood sample with the lysis composition. In some embodiments, a method comprises contacting a blood sample comprising an infectious agent (e.g., a bacteria or fungus) to a lysing composition, wherein all of the infectious agent is preserved after lysing the blood sample with the lysis composition. Preserved infectious agent, for example a preserved bacteria or fungus, may be phenotyped (e.g., using an image-based technique) after lysing. Preserved infectious agent, for example a preserved bacteria or fungus, may be cultured after lysing.

Lysis compositions provided herein may be sufficiently gentle to lyse blood cells (e.g., in amounts described elsewhere) while preserving colony-forming units (CFUs) of an infectious agent (e.g., instead of damaging, degrading, and/or lysing the infectious agent) such that a significant percentage of the colony-forming units are preserved after lysing. CFU preservation may be used as an indication of substantively unchanged phenotype. CFU preservation may be used as an indication that an infectious agent has been preserved. CFU preservation may be used as an indication that an infectious agent is intact. CFU preservation may be used as an indication that an infectious agent is viable. In some embodiments, a method comprises contacting a blood sample comprising an infectious agent (e.g., a bacteria or fungus) to a lysing composition, wherein at least 50% of colony-forming units (e.g., of the bacteria or fungus) in the blood sample are preserved after lysing the blood sample with the lysis composition. In some embodiments, a method comprises contacting a blood sample comprising an infectious agent (e.g., a bacteria or fungus) to a lysing composition, wherein at least 70% of colony-forming units (e.g., of the bacteria or fungus) in the blood sample are preserved after lysing the blood sample with the lysis composition. In some embodiments, a method comprises contacting a blood sample comprising an infectious agent (e.g., a bacteria or fungus) to a lysing composition, wherein at least 80% of colony-forming units (e.g., of the bacteria or fungus) in the blood sample are preserved after lysing the blood sample with the lysis composition. In some embodiments, a method comprises contacting a blood sample comprising an infectious agent (e.g., a bacteria or fungus) to a lysing composition, wherein at least 85% of colony-forming units (e.g., of the bacteria or fungus) in the blood sample are preserved after lysing the blood sample with the lysis composition. In some embodiments, a method comprises contacting a blood sample comprising an infectious agent (e.g., a bacteria or fungus) to a lysing composition, wherein at least 90% of colony-forming units (e.g., of the bacteria or fungus) in the blood sample are preserved after lysing the blood sample with the lysis composition. In some embodiments, a method comprises contacting a blood sample comprising an infectious agent (e.g., a bacteria, fungus) to a lysing composition, wherein at least 95% of colony-forming units (e.g., of the bacteria or fungus) in the blood sample are preserved after lysing the blood sample with the lysis composition. In some embodiments, a method comprises contacting a blood sample comprising an infectious agent (e.g., a bacteria or fungus) to a lysing composition, wherein at least 98% of colony-forming units (e.g., of the bacteria or fungus) in the blood sample are preserved after lysing the blood sample with the lysis composition. In some embodiments, a method comprises contacting a blood sample comprising an infectious agent (e.g., a bacteria or fungus) to a lysing composition, wherein at least 99% of colony-forming units (e.g., of the bacteria or fungus) in the blood sample are preserved after lysing the blood sample with the lysis composition. In some embodiments, a method comprises contacting a blood sample comprising an infectious agent (e.g., a bacteria or fungus) to a lysing composition, wherein at least 100% (e.g., all) colony-forming units (e.g., of the bacteria or fungus) in the blood sample are preserved after lysing the blood sample with the lysis composition. Preserved colony-forming units may be cultured after lysing. Preserved colony-forming units may be characterized [e.g., phenotyped (e.g., using an imaging-based method, for example a label-free microscopy technique)] after lysing.

In some embodiments, a colony-forming unit (CFU) recovery of an infectious agent (e.g., a bacteria or a fungus) is at least 50% after lysing a blood sample comprising the infectious agent with a lysis composition. In some embodiments, the colony-forming unit (CFU) recovery is at least 70%. In some embodiments, the colony-forming unit (CFU) recovery is at least 80%. In some embodiments, the colony-forming unit (CFU) recovery is at least 85%. In some embodiments, the colony-forming unit (CFU) recovery is at least 90%. In some embodiments, the colony-forming unit (CFU) recovery is at least 95%. In some embodiments, the colony-forming unit (CFU) recovery is at least 98%. In some embodiments, the colony-forming unit (CFU) recovery is at least 99%. In some embodiments, the colony-forming unit (CFU) recovery is at least 100% (e.g., is 100%). Colony-forming units may be cultured after lysing.

In some embodiments, a method comprises contacting a blood sample and a lysing composition to maintain viability of an infectious agent in the blood sample, wherein a colony-forming-unit (CFU) recovery of the infectious agent is at least 50%. In some embodiments, contacting a blood sample and a lysing composition comprises combining a blood sample and a lysing composition to maintain viability of an infectious agent in the blood sample, wherein a colony-forming unit (CFU) recovery of the infectious agent is at least 50%. In some embodiments, a colony-forming unit (CFU) recovery is at least 70%. In some embodiments, a colony-forming unit (CFU) recovery is at least 80%. In some embodiments, a colony-forming unit (CFU) recovery is at least 85%. In some embodiments, a colony-forming unit (CFU) recovery is at least 90%. In some embodiments, a colony-forming unit (CFU) recovery is at least 95%. In some embodiments, a colony-forming unit (CFU) recovery is at least 98%. In some embodiments, a colony-forming unit (CFU) recovery is at least 99%. In some embodiments, a colony-forming unit (CFU) recovery is at least 100% (e.g., is 100%).

In some embodiments, a composition is formed from a combination of a lysing composition comprising lysing agent and a blood sample comprising bacteria.

In some embodiments, lysing agent comprises (e.g., is) Triton X-100. In some embodiments, lysing agent comprises (e.g., is) IGEPAL CA-630. In some embodiments, lysing agent comprises (e.g., is) one or more saponins. In some embodiments, lysing agent comprises (e.g., is) Tween 20. In some embodiments, lysing agent comprises (e.g., is) Tween 80. In some embodiments, lysing agent comprises (e.g., is) sodium cholate. In some embodiments, lysing agent comprises (e.g., is) Pluronic F-127. In some embodiments, lysing agent comprises (e.g., further comprises) a secondary lysing agent. In some embodiments, a secondary lysing agent comprises (e.g., is) ammonium chloride and/or sodium chloride. In some embodiments, lysing agent comprises (e.g., further comprises) ammonium chloride and/or sodium chloride.

In some embodiments, a concentration of lysing agent in a lysing composition is no more than 10% (e.g., by volume). In some embodiments, a concentration of lysing agent in a lysing composition is no more than 5% (e.g., by volume). In some embodiments, a concentration of lysing agent in a lysing composition is no more than 4% (e.g., by volume). In some embodiments, a concentration of lysing agent in a lysing composition is no more than 3% (e.g., by volume). In some embodiments, a concentration of lysing agent in a lysing composition is no more than 2% (e.g., by volume). In some embodiments, a concentration of lysing agent in a lysing composition is no more than 1% (e.g., by volume). In some embodiments, a concentration of lysing agent in a lysing composition is in a range of from 0.1% to 3% (e.g., by volume). In some embodiments, a concentration of lysing agent in a lysing composition is in a range of from 0.5% to 3% (e.g., by volume). In some embodiments, a concentration of lysing agent in a lysing composition is in a range of from 0.1% to 2% (e.g., by volume). In some embodiments, a concentration of lysing agent in a lysing composition is in a range of from 0.5% to 2% (e.g., by volume). In some embodiments, a concentration of lysing agent in a lysing composition is in a range of from 0.1% to 1.5% (e.g., by volume). In some embodiments, a concentration of lysing agent in a lysing composition is in a range of from 0.25% to 1.5% (e.g., by volume). In some embodiments, a concentration of lysing agent in a lysing composition is in a range of from 0.5% to 1.5% (e.g., by volume). In some embodiments, a concentration of lysing agent in a lysing composition is in a range of from 0.1% to 1% (e.g., by volume). In some embodiments, a concentration of lysing agent in a lysing composition is in a range of from 0.5% to 1% (e.g., by volume). In some embodiments, a concentration of lysing agent in a lysing composition is at least 10% (e.g., by volume). In some embodiments, a concentration of lysing agent in a lysing composition is at least 15% (e.g., by volume). In some embodiments, a concentration of lysing agent in a lysing composition is at least 20% (e.g., by volume). In some embodiments, a concentration of lysing agent in a lysing composition is at least 25% (e.g., by volume). In some embodiments, a concentration of lysing agent in a lysing composition is no more than 50% (e.g., by volume). In some embodiments, a concentration of lysing agent in a total volume of a lysing composition and a blood sample is at least 0.1% and no more than 3% (e.g., by volume). In some embodiments, a concentration of lysing agent in a total volume of a lysing composition and a blood sample is at least 0.5% and no more than 3% (e.g., by volume). In some embodiments, a concentration of lysing agent in a total volume of a lysing composition and a blood sample is at least 0.4% and no more than 3% (e.g., by volume). In some embodiments, a concentration of lysing agent in a total volume of a lysing composition and a blood sample is at least 0.1% and no more than 2% (e.g., by volume). In some embodiments, a concentration of lysing agent in a total volume of a lysing composition and a blood sample is at least 0.5% and no more than 2% (e.g., by volume). In some embodiments, a concentration of lysing agent in a total volume of a lysing composition and a blood sample is at least 0.1% and no more than 1.5% (e.g., by volume). In some embodiments, a concentration of lysing agent in a total volume of a lysing composition and a blood sample is at least 0.25% and no more than 3% (e.g., by volume). In some embodiments, a concentration of lysing agent in a total volume of a lysing composition and a blood sample is at least 0.5% and no more than 1.5% (e.g., by volume). In some embodiments, a concentration of lysing agent in a total volume of a lysing composition and a blood sample is at least 0.1% and no more than 1% (e.g., by volume). In some embodiments, a concentration of lysing agent in a total volume of a lysing composition and a blood sample is at least 0.75% and no more than 1.25% (e.g., by volume). In some embodiments, a concentration of lysing agent in a total volume of a lysing composition and a blood sample is at least 0.5% and no more than 1% (e.g., by volume). In some embodiments, a concentration of lysing agent in a total volume of a lysing composition and a blood sample is 1% (e.g., by volume). In some embodiments, a concentration of lysing agent in a formed composition is at least 0.1% and no more than 3% (e.g., by volume). In some embodiments, a concentration of lysing agent in a formed composition is at least 0.5% and no more than 3% (e.g., by volume). In some embodiments, a concentration of lysing agent in a formed composition is at least 0.4% and no more than 3% (e.g., by volume). In some embodiments, a concentration of lysing agent in a formed composition is at least 0.1% and no more than 2% (e.g., by volume). In some embodiments, a concentration of lysing agent in a formed composition is at least 0.5% and no more than 2% (e.g., by volume). In some embodiments, a concentration of lysing agent in a formed composition is at least 0.1% and no more than 1.5% (e.g., by volume). In some embodiments, a concentration of lysing agent in a formed composition is at least 0.25% and no more than 3% (e.g., by volume). In some embodiments, a concentration of lysing agent in a formed composition is at least 0.5% and no more than 1.5% (e.g., by volume). In some embodiments, a concentration of lysing agent in a formed composition is at least 0.1% and no more than 1% (e.g., by volume). In some embodiments, a concentration of lysing agent in a formed composition is at least 0.75% and no more than 1.25% (e.g., by volume). In some embodiments, a concentration of lysing agent in a formed composition is at least 0.5% and no more than 1% (e.g., by volume). In some embodiments, a concentration of lysing agent in a formed composition is 1% (e.g., by volume).

In some embodiments, a composition has been formed from a combination of a blood sample and a lysing composition in a ratio in a range of from 3:1 to 30:1 (e.g., 4:1 or 24:1). In some embodiments, a ratio is in a range of from 3:1 to 5:1. In some embodiments, a ratio is in a range of from 22:1 to 26:1. In some embodiments, a composition has been formed from a combination of a blood sample and a lysing composition such that a blood sample is at least 75% (e.g., at least 80%, at least 85%, at least 90%, or at least 95%) of total volume thereof. In some embodiments, a composition has been formed from a combination of a blood sample and a lysing composition in a ratio such that lysing agent from a lysing composition is present in a concentration of at least 0.4% and no more than 3% (e.g., by volume). In some embodiments, a composition has been formed from a combination of a blood sample and a lysing composition in a ratio such that lysing agent from a lysing composition is present in a concentration of at least 0.5% and no more than 1.5% (e.g., by volume). In some embodiments, a composition has been formed from a combination of a blood sample and a lysing composition in a ratio such that lysing agent from a lysing composition is present in a concentration of at least 0.75% and no more than 1.25% (e.g., by volume). In some embodiments, a composition has been formed from a combination of a blood sample and a lysing composition such that a lysing composition is no more than 25% (e.g., no more than 20%, no more than 15%, no more than 10%, or no more than 5%) of total volume thereof.

Combinations (e.g., mixtures) of blood samples and lysing compositions may be at high lysing composition to blood sample ratios instead. Using a high ratio of lysing composition to blood sample may offer several advantages. First, this may enable effective lysis even when a lower volume of blood is available, which may be crucial when sample is limited. Second, this approach may facilitate processing a higher blood volume per unit time, which may improve the speed of diagnostics. Higher lysing composition to blood sample ratios may be used in places of lower ratios to achieve similar, same, or higher throughput in terms of amount of sample lysed per unit time. In some embodiments, a composition has been formed from a combination of a blood sample and a lysing composition in a ratio of blood sample to lysing composition in a range of from 1:30 to 1:1 (e.g., 1:3, 1:5, 1:10, 1:20). In some embodiments, a ratio of blood sample to lysing composition is in a range of from 1:20 to 1:2. In some embodiments, a ratio of blood sample to lysing composition is in a range of from 1:15 to 1:2. In some embodiments, a ratio of blood sample to lysing composition is in a range of from 1:10 to 1:2. In some embodiments, a ratio of blood sample to lysing composition is in a range of from 1:10 to 1:3. In some embodiments, a ratio of blood sample to lysing composition is in a range of from 1:5 to 1:2. In some embodiments, a composition has been formed from a combination of a blood sample and a lysing composition such that a blood sample is at least 75% (e.g., at least 80%, at least 85%, at least 90%, or at least 95%) of total volume thereof. In some embodiments, a composition has been formed from a combination of a blood sample and a lysing composition in a ratio such that lysing agent from a lysing composition is present in a concentration of at least 0.4% and no more than 3% (e.g., by volume). In some embodiments, a composition has been formed from a combination of a blood sample and a lysing composition in a ratio such that lysing agent from a lysing composition is present in a concentration of at least 0.5% and no more than 1.5% (e.g., by volume). In some embodiments, a composition has been formed from a combination of a blood sample and a lysing composition in a ratio such that lysing agent from a lysing composition is present in a concentration of at least 0.75% and no more than 1.25% (e.g., by volume). In some embodiments, a composition has been formed from a combination of a blood sample and a lysing composition such that a lysing composition is no more than 25% (e.g., no more than 20%, no more than 15%, no more than 10%, or no more than 5%) of total volume thereof.

In some embodiments, a bacteria comprises a strain of ESKAPE-E bacteria (e.g., Enterococcus faecalis, Staphylococcus aureus, Klebsiella quasipneumoniae, Acinetobacter baumannii, Pseudomonas aeruginosa, Escherichia coli, or Enterobacter spp.). In some embodiments, a bacteria comprises a strain of Escherichia coli. In some embodiments, a bacteria comprises Gram-negative bacteria. In some embodiments, a bacteria comprises Gram-positive bacteria. In some embodiments, a formed composition is substantially devoid of red blood cells (e.g., such that culturing and/or identification and/or AST can be performed using a microfluidics device). In some embodiments, a composition is substantially devoid of blood cells (e.g., red blood cells and/or white blood cells) (e.g., such that culturing and/or identification and/or AST can be performed using a microfluidics device). In some embodiments, a composition comprises viable bacteria and is substantially devoid of red blood cells.

In some embodiments, a composition derived from a whole blood sample, wherein a composition comprises one or more viable infectious agents and is substantially devoid of blood cells (e.g., red blood cells and/or white blood cells) (e.g., such that culturing and/or identification and/or AST can be performed using a microfluidics device).

In some embodiments, a composition is derived from a whole blood sample, wherein a composition comprises one or more viable bacteria and is substantially devoid of blood cells (e.g., red blood cells and/or white blood cells) (e.g., such that culturing and/or identification and/or AST can be performed using a microfluidics device).

In some embodiments, a composition is derived from a whole blood sample, wherein a composition comprises one or more viable fungi and is substantially devoid of blood cells (e.g., red blood cells and/or white blood cells) (e.g., such that culturing and/or identification and/or AST can be performed using a microfluidics device).

In some embodiments, a blood sample has a volume in a range of from 0.01 mL to 100 mL (e.g., of from 0.05 mL to 50 mL, of from 0.5 mL to 25 mL, of from 0.5 mL to 10 mL). In some embodiments, a lysing composition has a volume in a range of from 0.001 mL to 10 mL. In some embodiments, a lysed blood composition has a volume in a range of from 0.01 mL to 100 mL (e.g., of from 0.05 mL to 50 mL, of from 0.5 mL to 25 mL, of from 0.5 mL to 10 mL).

The foregoing description is primarily directed to methods and compositions for lysing blood while preserving one or more infectious agents in the blood, for example to allow for further analysis (e.g., phenotyping) to be performed. The present disclosure recognizes that the selective lysing that occurs between blood cells and infectious agent(s) (e.g., bacterial and/or fungal cells) can be a result of differences in mechanical properties between blood cells and infectious agent(s) (e.g., bacterial cells and/or fungal cells). For examples, blood cells may be relatively soft and/or deformable, while bacteria and fungi cells may be relatively hard and/or rigid such that they respond differently to contact with a lysing composition. Likewise, other cells present in a blood sample that are relatively rigid and/or hard compared to blood cells may be selectively preserved during lysis of blood cells in the blood sample by contact (e.g., mixing) with a lysing composition disclosed herein. Such other cells may be rare cells and/or non-healthy cells, such as cancer cells and/or sickle cells. Thus, the present disclosure expressly contemplates embodiments of methods and compositions for lying blood samples that comprise rare cells and/or non-healthy cells that are analogous (e.g., equivalent) to methods and compositions disclosed herein previously with respect to lysing blood samples that comprise one or more infectious agents. Additional embodiments of methods and compositions for lysing a blood sample that comprises both (i) one or more infectious agents and (ii) one or more rare cells and/or one or more non-healthy cells are also contemplated. In some embodiments, a blood sample (e.g., fresh blood) (e.g., whole blood) that comprises rare cells and/or non-healthy cells may similarly be selectively lysed to preserve rare cells and/or non-healthy cells present in the sample using methods and compositions disclosed herein.

In some embodiments, a method comprises contacting a blood sample (e.g., fresh blood sample) (e.g., whole blood sample) comprising one or more rare cells and/or one or more non-healthy cells to a lysing composition thereby obtaining a lysed blood composition comprising the one or more rare cells and/or non-healthy cells. In some embodiments, one or more rare cells and/or one or more non-healthy cells comprise one or more cancer cells. In some embodiments, one or more rare cells and/or one or more non-healthy cells comprise one or more sickle cells. In some embodiments, one or more rare cells and/or one or more non-healthy cells comprise one or more cells with genetic mutations. In some embodiments, one or more rare cells and/or one or more non-healthy cells comprise one or more lymphocytes (e.g., T cells, B cells, or natural killer cells). In some embodiments, one or more rare cells and/or one or more non-healthy cells comprise one or more circulating tumor cells. In some embodiments, one or more rare cells and/or one or more non-healthy cells comprise one or more cancer stem cells. In some embodiments, one or more rare cells and/or one or more non-healthy cells comprise one or more circulating endothelial cells. The lysing composition may be a lysing composition disclosed herein elsewhere.

### EXAMPLES

The following examples are provided as illustrations and are not intended to be limiting with respect to any subject matter disclosed herein.

### Example 1: Whole Blood Sample Preparation

The present example describes lysis compositions (referred to in this example as “lysis buffers”) of the present disclosure that were tested to determine lysing performance, for example as indicated by lysing efficiency of blood cells and infectious agent viability after lysing.

### Lysis Assay Properties

Lysis compositions tested in this example may be used as part of a rapid, cost-effective, efficient sample preparation in a framework of clinical diagnostics of sepsis. In contrast, typical blood culture takes approximately 3-5 days.

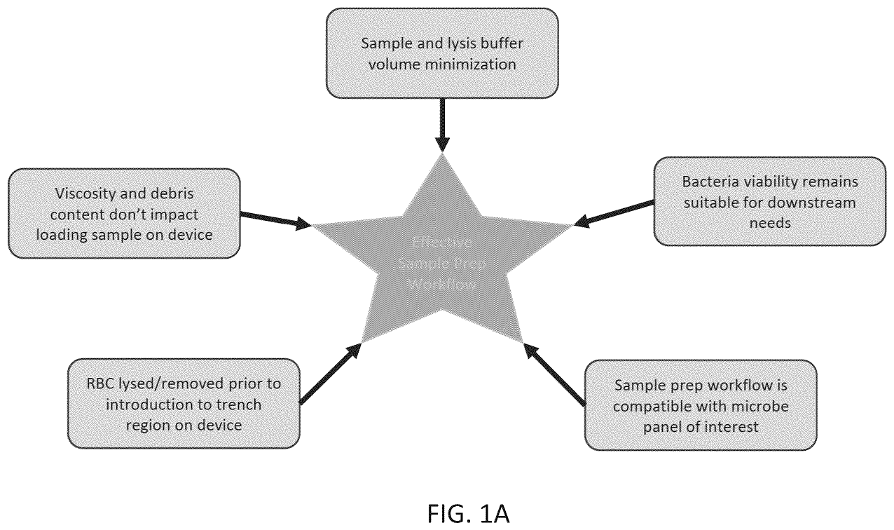

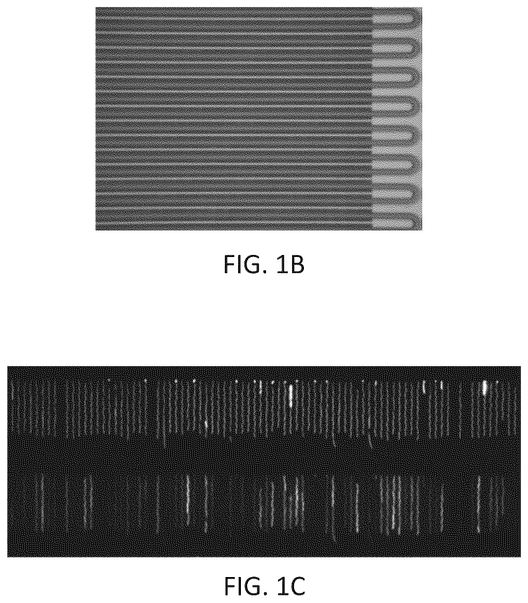

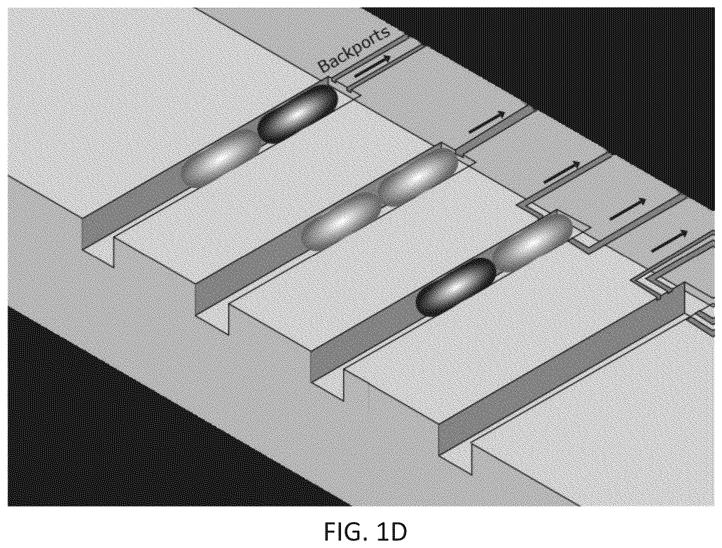

Blood components (e.g., RBC, platelets, white blood cells) in a sample may be removed using chemical and/or physical means. FIG. 1A outlines further properties of a sample preparation workflow with the accompanied insights. First, minimal sample and lysis buffer volumes may be used, which translates into cost-effect and efficient testing. Second, the resulting sample has viscosity values suitable for the sample injection into a microfluidic chip for further analysis. One function of lysing RBC is to ensure no clogging of a microfluidic device occurs, for example since fragmented blood cells and debris may not clog such a device. Third, the timing of the workflow is engineered to ensure that RBCs are lysed prior to reaching a sensing region (e.g., trenches) of the microfluidics device. Fourth, while lysing RBCs, viability of infectious agent (e.g., bacteria) that is present in a sample remains high (e.g., suitable for downstream needs of the analysis). Fifth, the workflow is compatible with various types of bacteria (e.g., gram-positive, gram negative, aerobic, anaerobic) and fungi.

### Microfluidic System

As shown in FIGS. 1B-1D, a microfluidic device miniaturizes blood culture at single-level by separating bacteria into individual channels with subsequent culturing of individual pathogenic lineages. The geometry leads to the ability of bacterial pathogenic identification and antimicrobial susceptibility testing. The technology is also scalable to potentially screen hundreds of patients per day and distinguish thousands of pathogens.

### Optimal RBC Lysis Buffer

In this Example, samples were typically exposed to a lysis buffer using the following recipes: stock lysis solutions were prepared in water (25% stock), 240 μL of a sample were mixed with 10 μL of a stock lysis solution, resulting in 1% final lysis buffer concentration; 90 μL of a sample were mixed with 10 μL of a stock lysis solution, resulting in 2.5% final lysis buffer concentration. The exposure was quenched with 1× phosphate-buffered saline (PBS) buffer after a certain time. The percentage of RBC lysis was calculated by measuring RBC number before and after exposure using a hemocytometer. Experiments were repeated in triplicates. Control experiments were performed with samples in water without any lysis buffer.

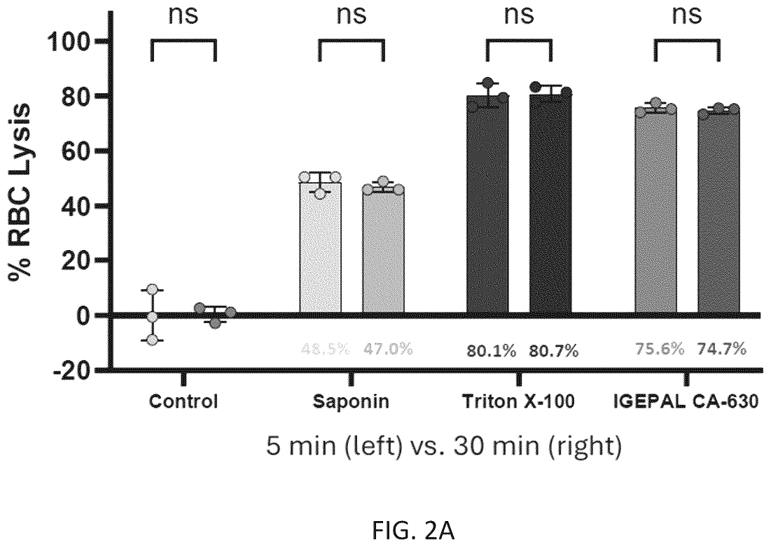

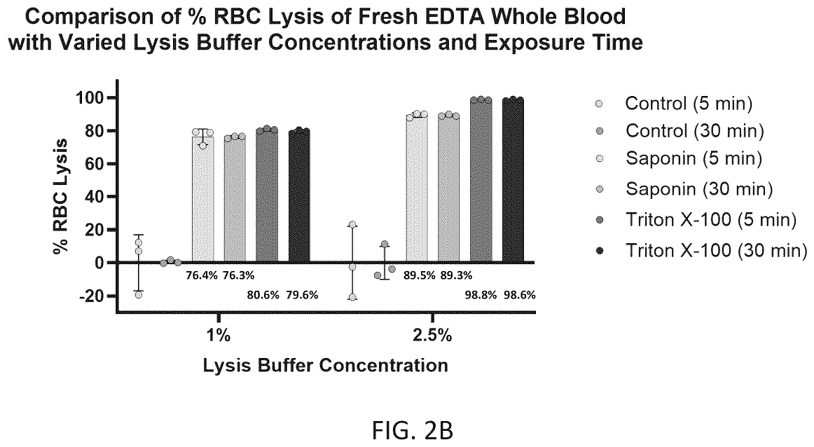

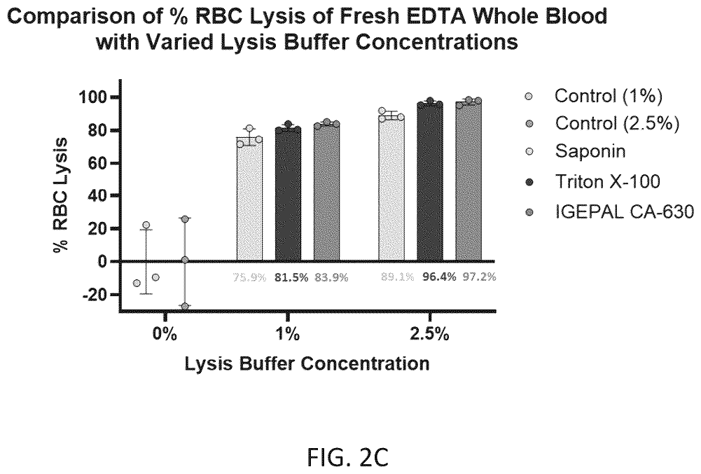

As shown in FIG. 2A, different lysis buffers of the present disclosure lyse RBCs of frozen EDTA whole blood samples. The data of FIG. 2A indicates that RBC lysis percentage did not change between 5 and 30 min of exposure (“ns” denotes no significant different). Therefore, exemplary lysis buffers of the present disclosure lyse RBCs within minutes and lysing percentage did not improve with time. FIG. 2B shows RBC lysis percentage at 5 minutes and 30 minutes for fresh EDTA whole blood at different concentrations of lysis buffers. The data of FIG. 2B also indicates that at different concentrations of lysis buffers, RBC lysis percentage did not appreciably change between 5 and 30 minutes of exposure. FIG. 2C shows RBC lysis percentage at 5 minutes for fresh EDTA whole blood at different concentrations of lysis buffers.

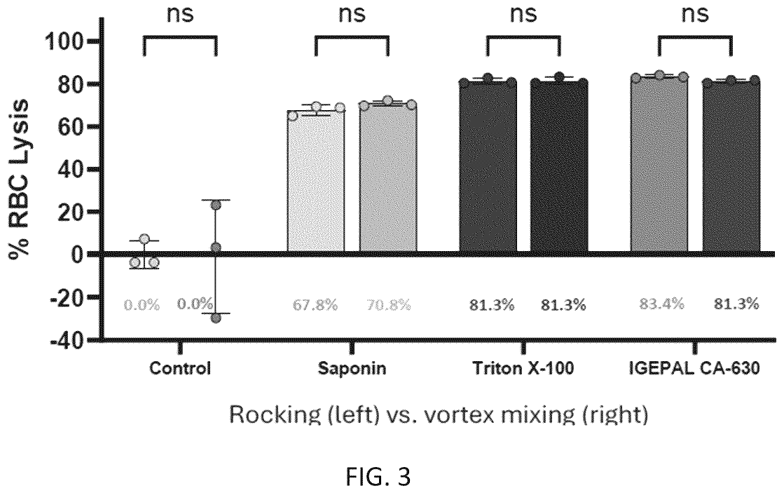

As shown in FIG. 3, lysing process of the present disclosure was combined with physical mixing, such as rocking and vortex mixing. Specifically, rocking was performed for 5 min and vortex mixing—for 30 seconds in the presence of lysis buffer used. As a result, samples showed no significant increase in RBC lysis percentage for lysis buffers used.

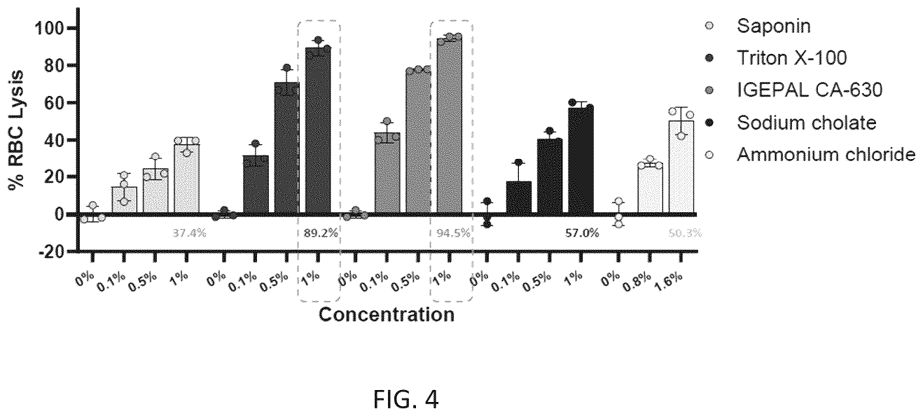

As shown in FIG. 4, different lysis buffers at different concentrations lyse different RBC percentage of frozen EDTA whole blood samples. For example, 1% IGEPAL CA-630 lysed 94.5% of RBCs, while 1% saponin buffer lysed 37.4% of RBCs. The data indicates 1% IGEPAL CA-630 and 1% Triton X-100 are the best performing lysing compositions among tested for these frozen EDTA whole blood samples.

### Effect of Ammonium Chloride

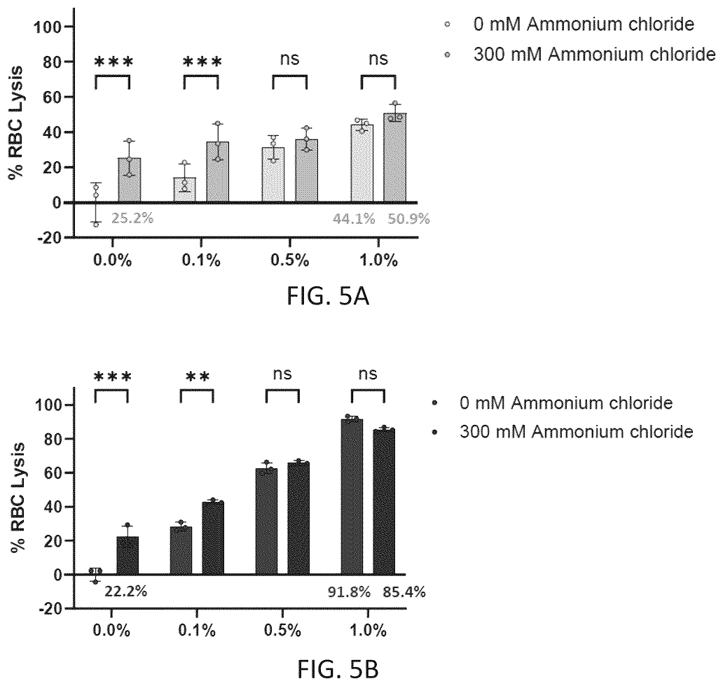

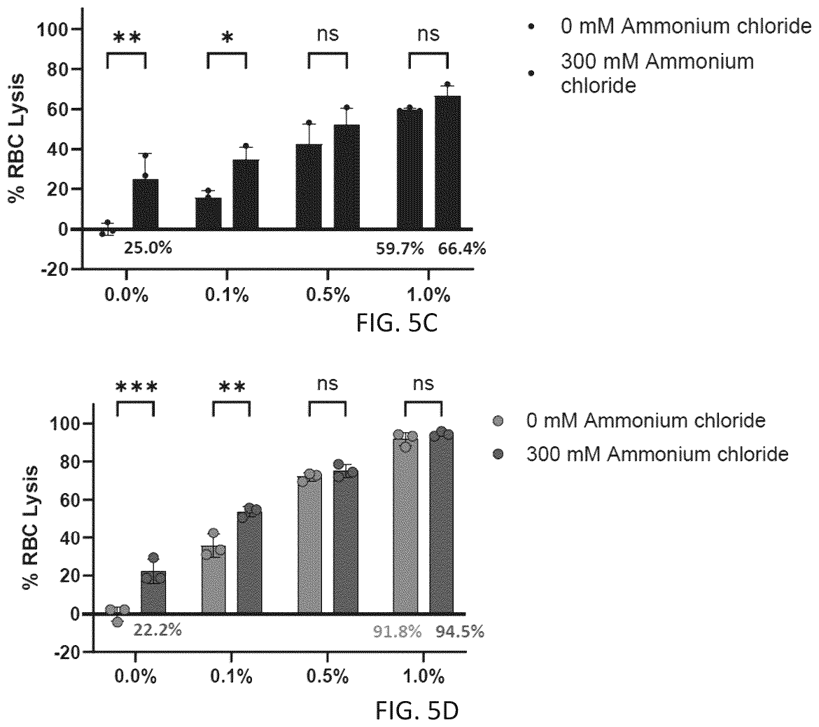

As shown in FIGS. 5A-5D, the addition of ammonium chloride to lysis buffers affects RBC lysed percentage of frozen EDTA whole blood samples. For example, as shown in FIG. 5A, adding 300 mM ammonium chloride to a water control increases lysed RBCs from 0 to 25.2%, while the same addition of 300 mM ammonium chloride to 1.0% saponin buffer results in similar values of 44.1% to 50.9% RBC lysed, respectively. Similarly, as shown in FIG. 5D, adding 300 mM ammonium chloride to a water control increases lysed RBC from 0 to 22.2%, while the same addition of 300 mM ammonium chloride to 1.0% IGEPAL CA-630 buffer does not change lysed RBCs: 91.8% and 94.5%, respectively. The data suggests that primary lysing agent works in tandem with ammonium chloride in the process of lysing cells and that at high detergent concentrations, approximately 0.5-1%, the impact of 300 mM ammonium chloride was not significant and lysis approaches saturation levels.

### Altering Sample Volume

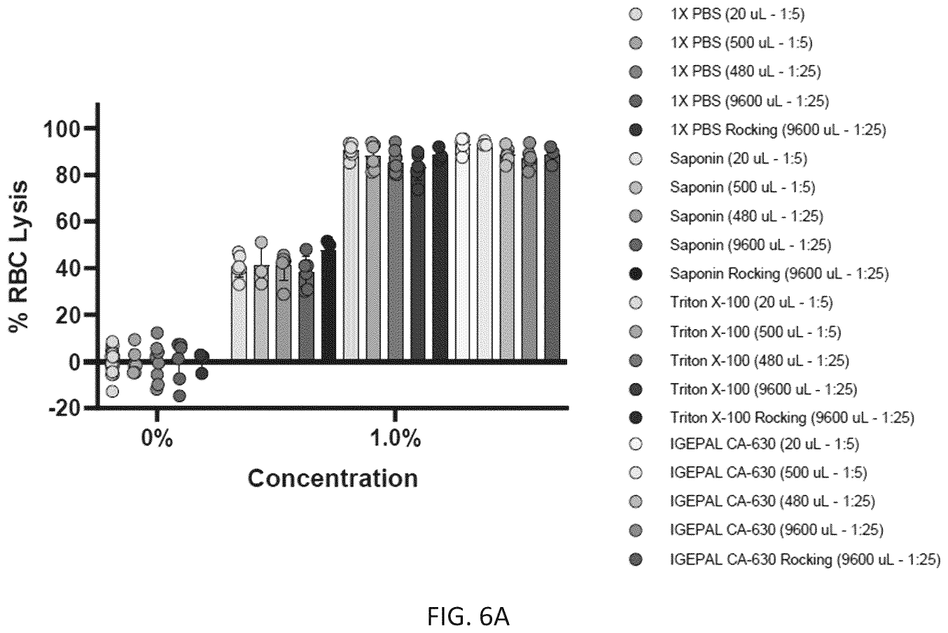

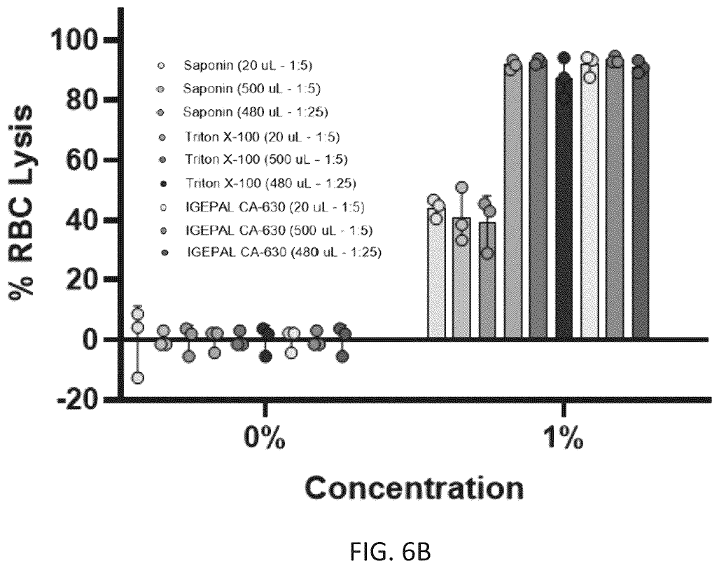

As shown in FIGS. 6A-6B, sample volume shows no significance in effectiveness of RBC lysis buffer in certain cases. Various sample volumes were tested from 20 μL up to 9.6 mL of frozen whole blood as treated with different lysis buffers at 1% concentrations in water, resulting in either 1:5 or 1:25 lysis buffer volume dilution (with whole blood sample). As shown in FIG. 6B, various sample volumes were tested from 20 μL up to 480 μL of frozen whole blood as treated with three lysis buffers at 1% concentrations in water. A comparison between the previously established 1:5 dilution of lysis buffer stock (20 μL sample) was compared to a 1:5 and 1:25 dilution of lysis buffer stock (500 μL sample and 480 μL sample, respectively), all with a final lysis buffer concentration of 1%. The data demonstrate the sample protocol can process larger sample volume that achieves more than 90% RBC lysis. The data also shows that lysis buffers of Triton X-100 or IGEPAL CA-630 (1% in water) can achieve more than 90% RBC lysis, regardless of lysis buffer dilution used. Certain test sample compositions and percentage RBC lysis results are summarized in Table 1. For example, exposure to Triton X-100 of 20 μL at the dilution ratio of 1:5 and of 9600 μL at the dilution ratio of 1:25 resulted in 90.5% and 83.4% RBC lysis, respectively. The data demonstrates that lysis buffer is the main lysis driver and not buffer dilution effects. Additionally, certain lysis compositions tested allow for minimal sample dilutions, which may improve total assay time (TAT).

TABLE 1
Test sample compositions and averaged
percentage RBC lysis values.

| | Blood volume | Lysis Buffer | % RBC lysis |
|---|---|---|---|
| Lysis Buffer | (μL) | Dilution Ratio | (Avg.) |
| Saponin | 20 | 1:5 | 40.8 |
| Saponin | 500 | 1:5 | 41.0 |
| Saponin | 480 | 1:25 | 41.0 |
| Saponin | 9600 | 1:25 | 38.1 |
| Saponin (rocking) | 9600 | 1:25 | 47.5 |
| Triton X-100 | 20 | 1:5 | 90.5 |
| Triton X-100 | 500 | 1:5 | 88.0 |
| Triton X-100 | 480 | 1:25 | 85.5 |
| Triton X-100 | 9600 | 1:25 | 83.4 |
| Triton X-100 | 9600 | 1:25 | 88.9 |
| (rocking) | | | |
| IGEPAL CA-630 | 20 | 1:5 | 93.1 |
| IGEPAL CA-630 | 500 | 1:5 | 93.5 |
| IGEPAL CA-630 | 480 | 1:25 | 88.7 |
| IGEPAL CA-630 | 9600 | 1:25 | 86.9 |
| IGEPAL CA-630 | 9600 | 1:25 | 88.6 |
| (rocking) | | | |

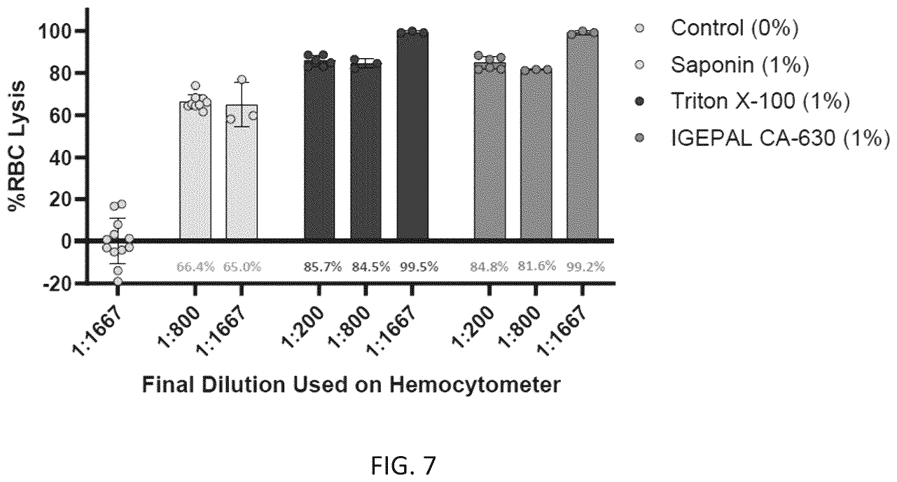

As shown in FIG. 7, lysis assays with various buffers perform well with fresh EDTA whole blood samples. For example, sample exposure to 1% final concentration IGEPAL CA-630 at 1:25 dilution ratio resulted in 85.7%, 84.5%, and 99.5% RBC lysis for 1:200, 1:800, and 1:1667 hemocytometer dilutions, respectively. Lysis results observed with fresh blood samples were lower with Triton X-100 and IGEPAL CA-630 than similar results for frozen blood samples, possibly due to more concentrated centrifuged blood. High dilutions with Triton X-100 and IGEPAL CA-630 may artificially increase lysis percentage due to over-dilution of a sample. Lower dilutions may demonstrate more statistically significant RBC values.

### Bacteria Stability

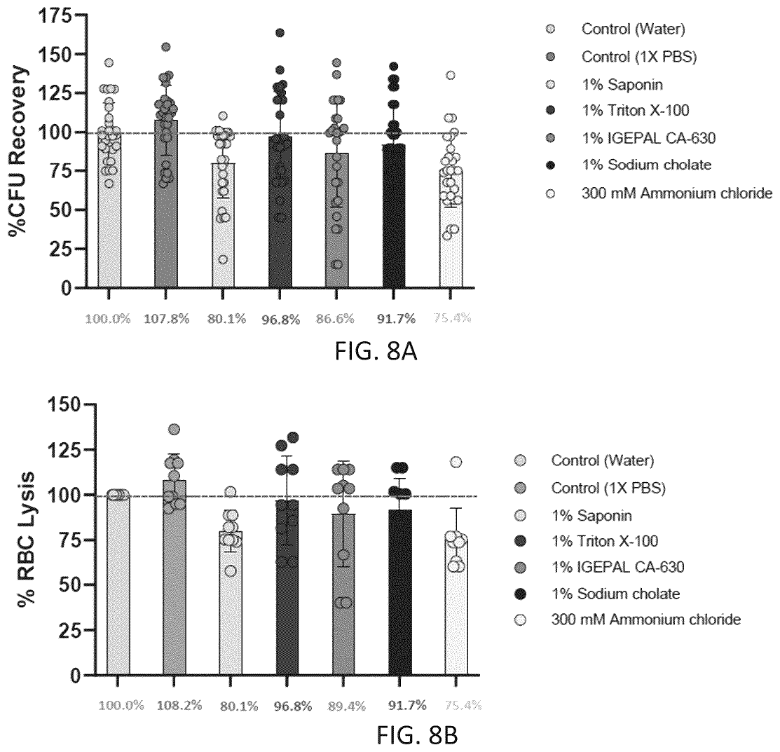

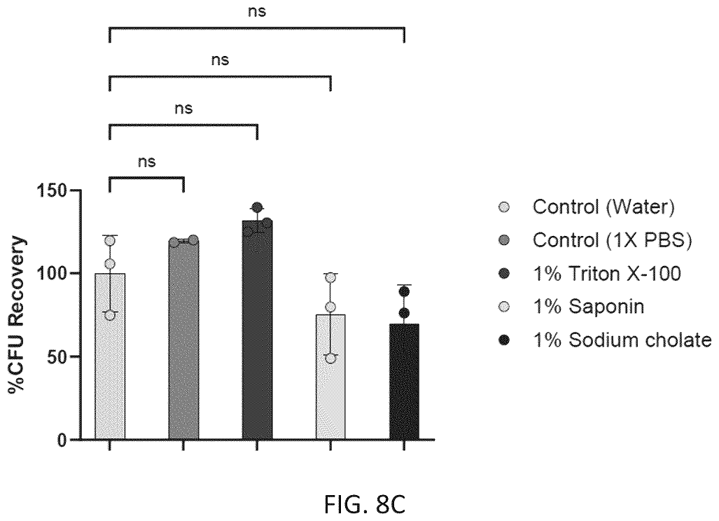

As shown in FIGS. 8A-8C, bacteria E. coli K-12 (ATCC 10798) samples remain stable in the lysis buffers at 1:5 dilution ratio of lysis buffer volume—lysis buffers that were found effective in lysing RBCs. For example, as shown in FIG. 8A, CFU recovery for bacteria exposed to 1% Triton X-100 and IGEPAL CA-630 were 96.8% and 86.8%, respectively. FIGS. 8A-8B compare results obtained at two different days, where FIG. 8A plots all individual plates and FIG. 8B plots mean values of an experiment. The noise present in experiments may be associated with low CFU numbers on plates for some experiments. Bacteria viability was measured by counting recovered CFU on agar plates. As shown in FIG. 8C, 1% Triton X-100 is the best performing lysis buffer among tested for CFU recovery for bacteria E. coli K-12 (ATCC 10798). Viability remains intact with Triton X-100 lysis buffer after 5 minutes of exposure time.

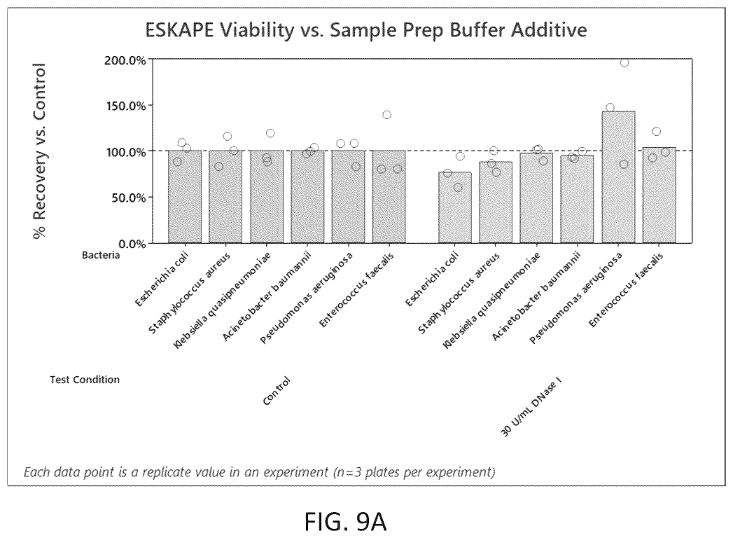

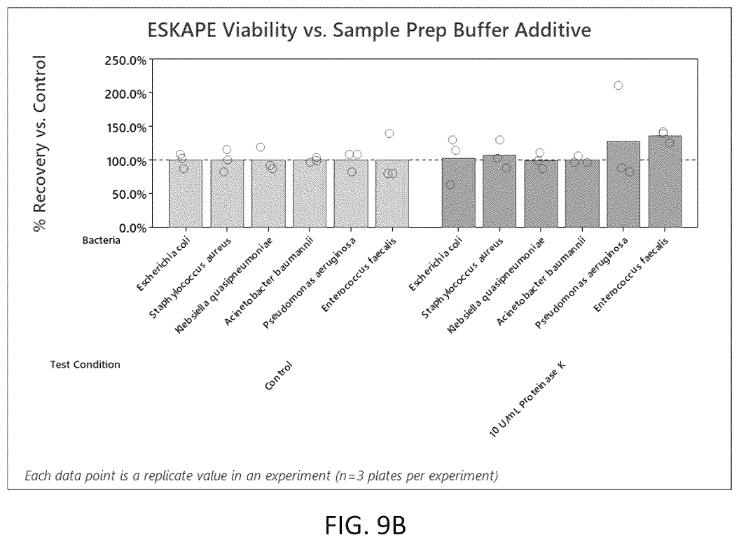

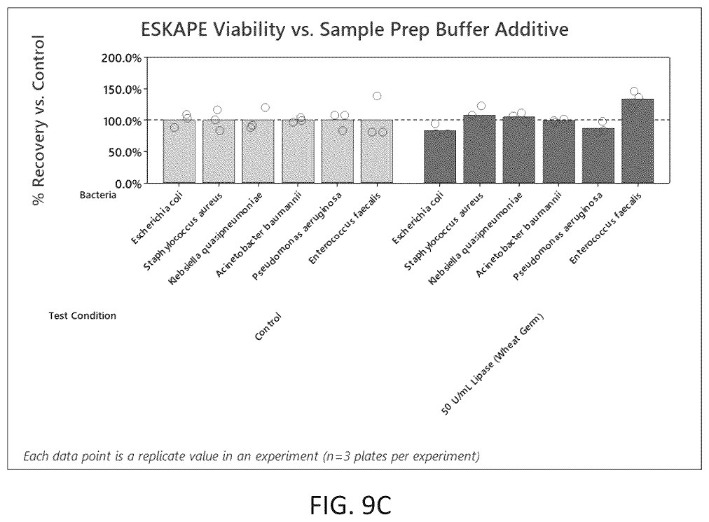

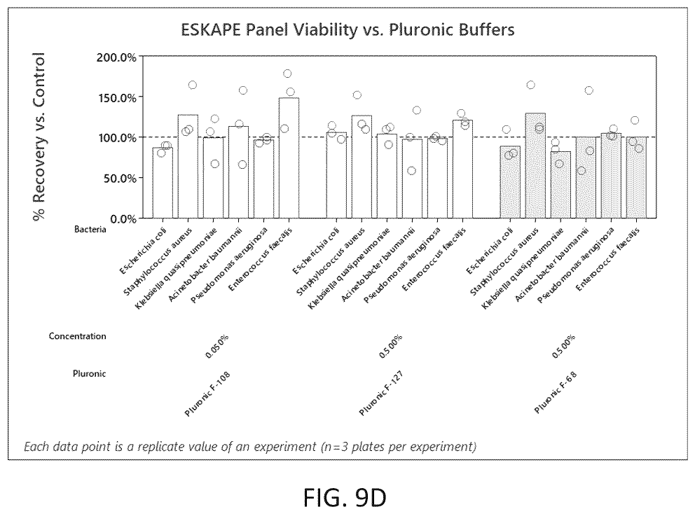

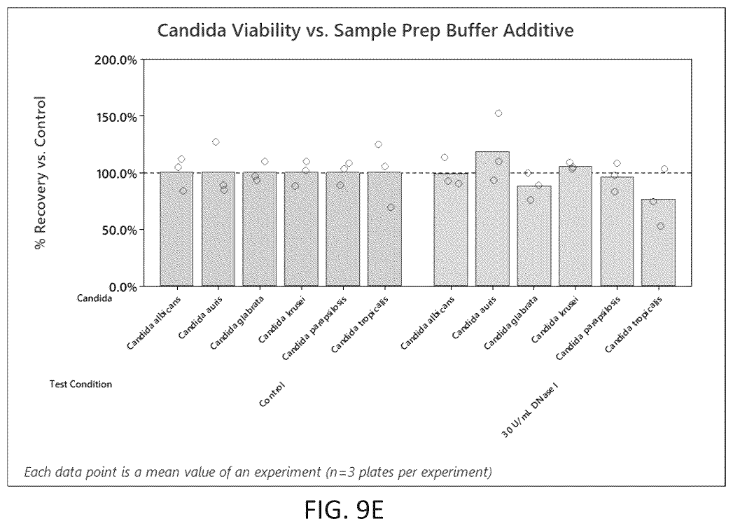

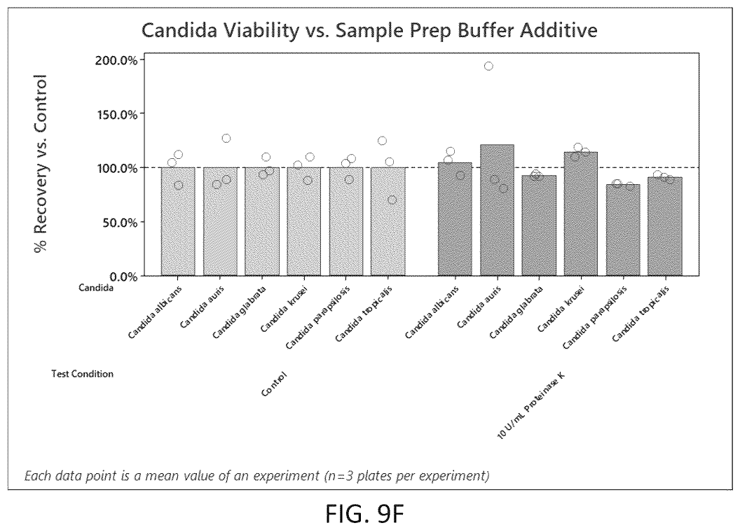

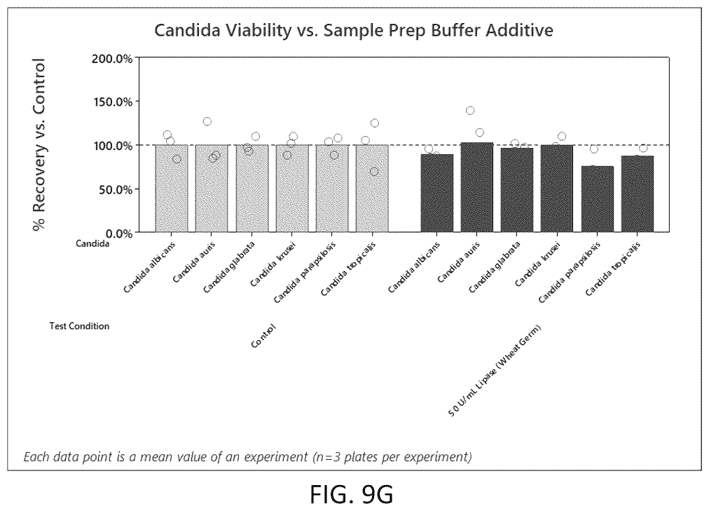

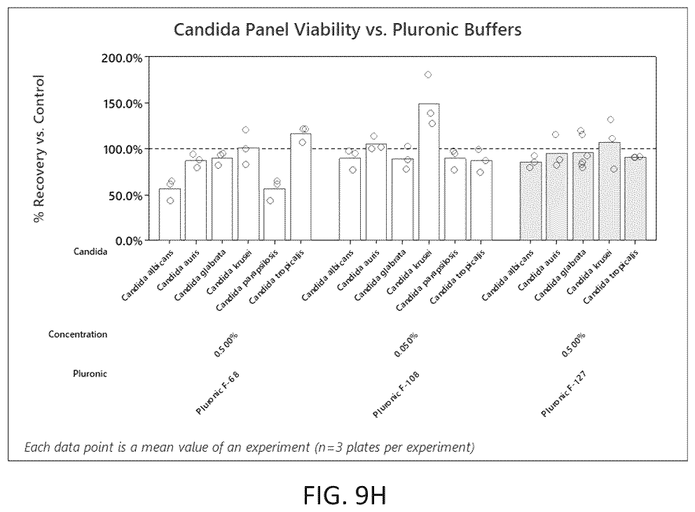

As shown in FIGS. 9A-9D, representative bacteria samples also remain largely viable (e.g., >75%) after exposure to different poloxamers for 15 minutes (FIG. 9D) and enzymes for 5 minutes (FIGS. 9A-9C). Bacteria viability was measured by counting recovered CFU on agar plates.

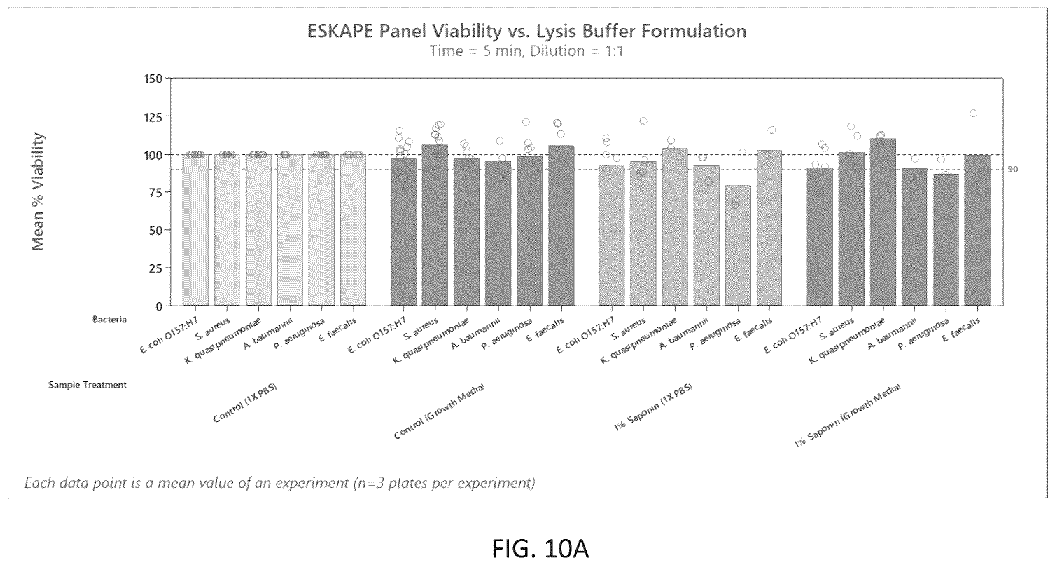

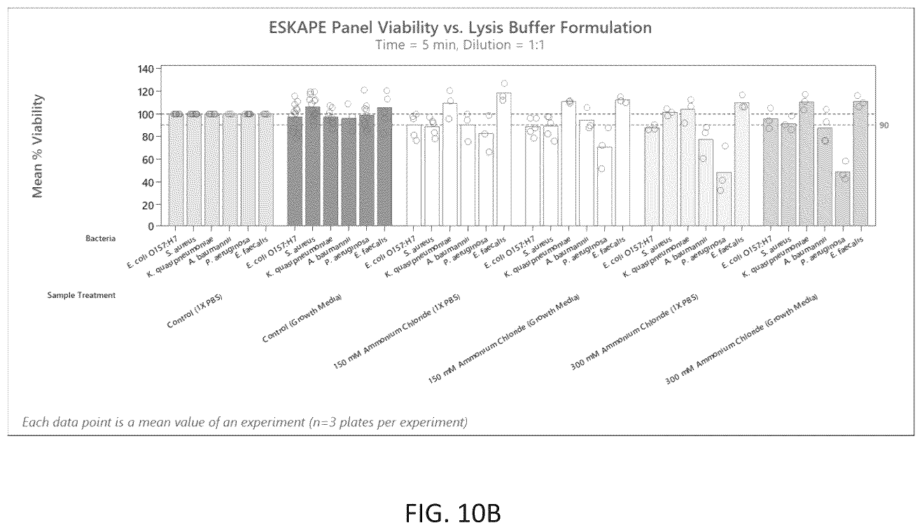

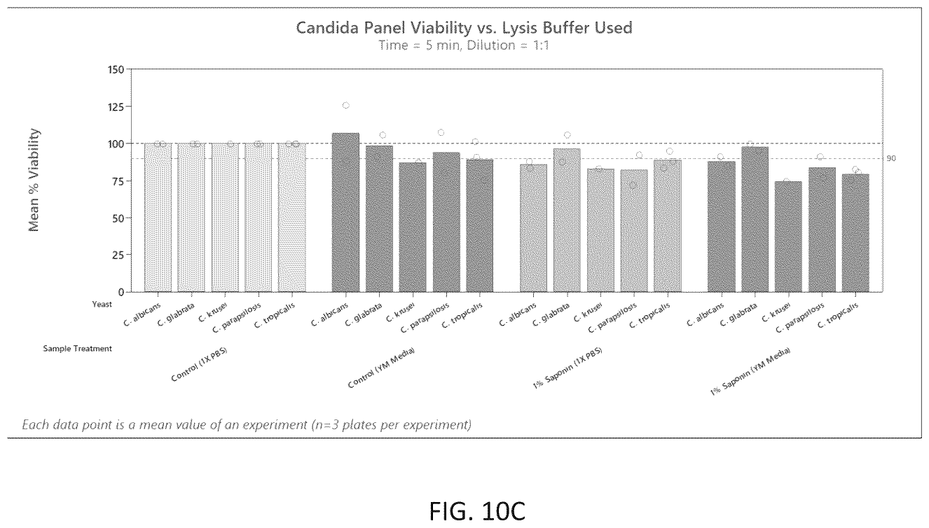

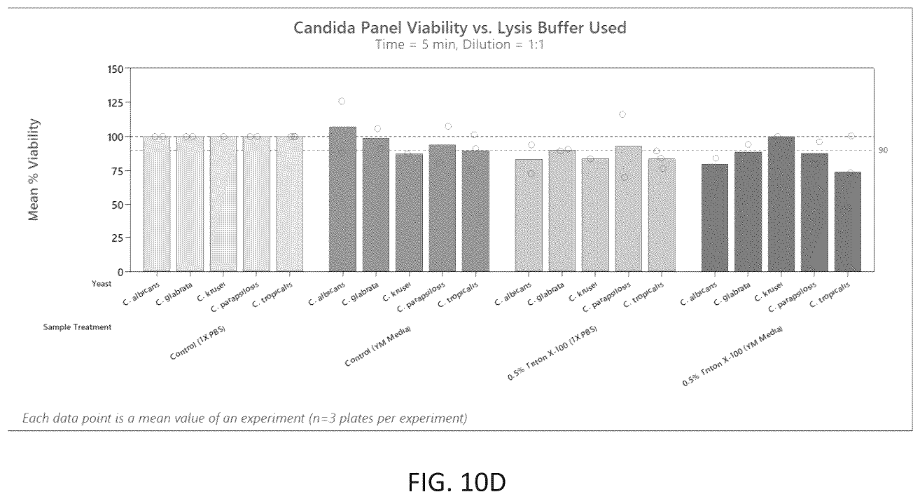

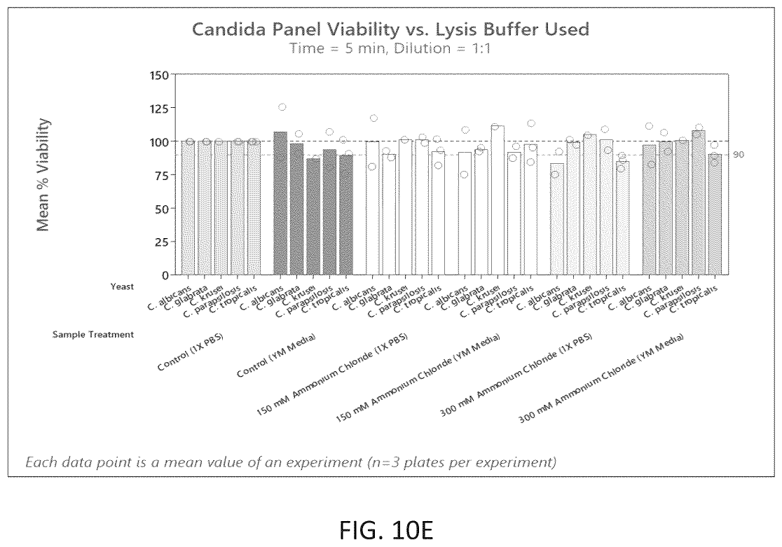

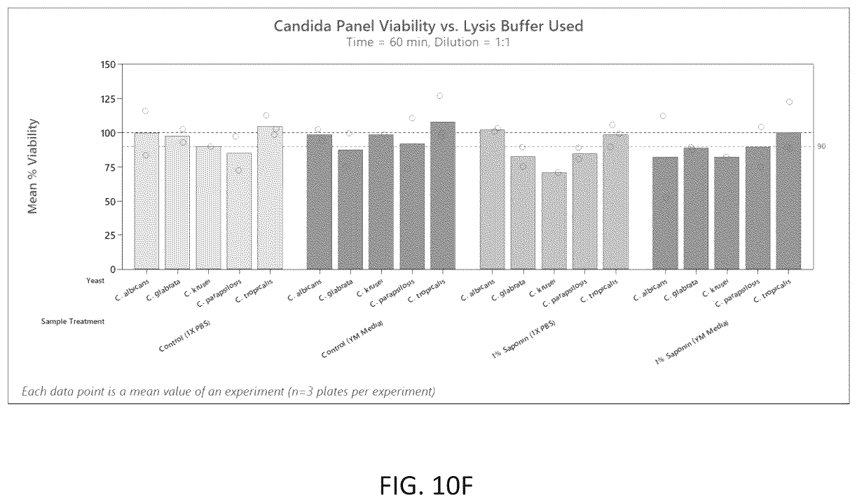

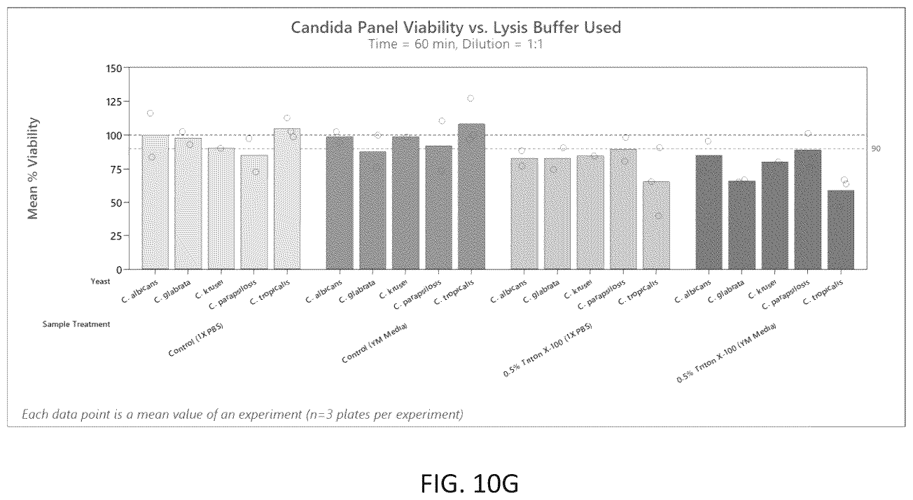

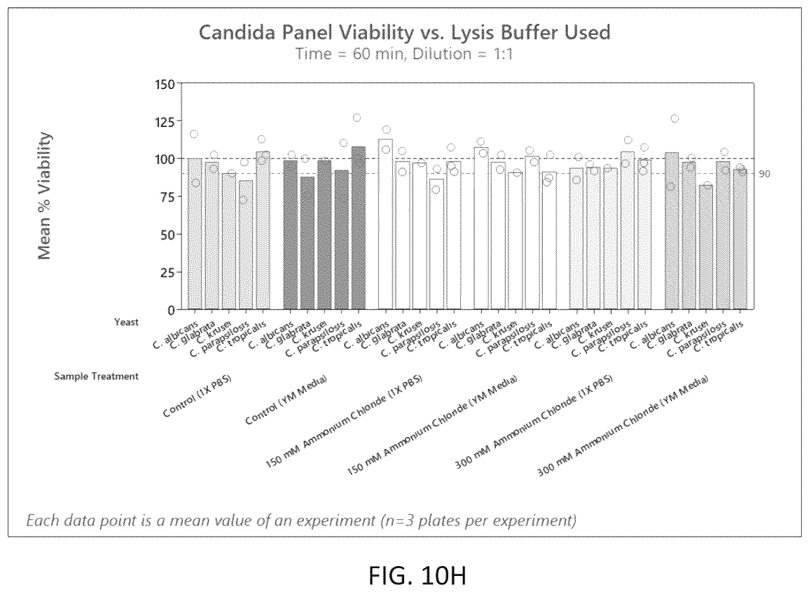

As shown in FIG. 10A, representative bacteria samples for each member of the ESKAPE panel (Escherichia coli, Staphylococcus aureus, Klebsiella quasipneumoniae, Acinetobacter baumannii, Pseudomonas aeruginosa, Enterococcus faecalis) also remain largely viable (e.g., >75%) after 5 minutes of exposure to saponin at 1% concentration in PBS or bacteria specific growth media (LB broth for Escherichia coli, Staphylococcus aureus; Nutrient broth for Klebsiella quasipneumoniae, Acinetobacter baumannii; Tryptic Soy broth for Pseudomonas aeruginosa; Brain Heart Infusion broth for Enterococcus faecalis). FIG. 10B also shows that representative bacteria samples for each member of the ESKAPE panel remain largely viable (e.g., >75%) after 5 minutes of exposure to ammonium chloride lysis buffers at 150 mM and 300 mM concentrations in PBS or bacteria specific growth media (LB broth for Escherichia coli, Staphylococcus aureus; Nutrient broth for Klebsiella quasipneumoniae, Acinetobacter baumannii; Tryptic Soy broth for Pseudomonas aeruginosa; Brain Heart Infusion broth for Enterococcus faecalis). CFU recovery percentage was determined by comparing data to the control CFU recovery in 1×PBS.

### Fungi Stability

As shown in FIGS. 9E-9H, representative fungi samples of various Candida spp. (Candida albicans, Candida auris, Candida glabrata, Candida krusei, Candida parapsilosis, or Candida tropicalis) also remain largely viable (>75%) (with the exception of some strains exposed to high concentrations of Pluronic F-68) after exposure to different poloxamers for 15 minutes and enzymes for 5 minutes. Candida sample viability was measured by counting recovered CFU on agar plates. Pluronic F-127 and Pluronic F-108 performed best.

As shown in FIGS. 10C-10H, samples for representative members of a Candida panel (Candida albicans, Candida glabrata, Candida krusei, Candida parapsilosis, Candida tropicalis) remain largely viable (e.g., >75%) up to 60 minutes (e.g., 5 minutes, 60 minutes) of exposure to saponin at 1% concentration in PBS or YM growth media, Triton X-100 at 0.5% concentration in PBS or YM growth media, or ammonium chloride at 150 mM and 300 mM concentrations in PBS or YM growth media. CFU recovery percentage was determined by comparing data to the control CFU recovery in 1×PBS.

RBC Recovery from Centrifuged Samples

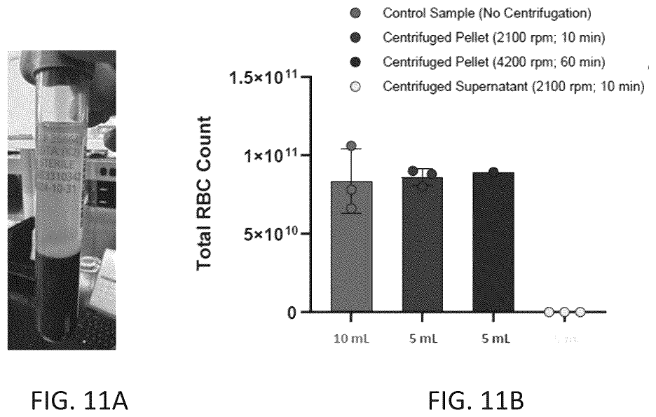

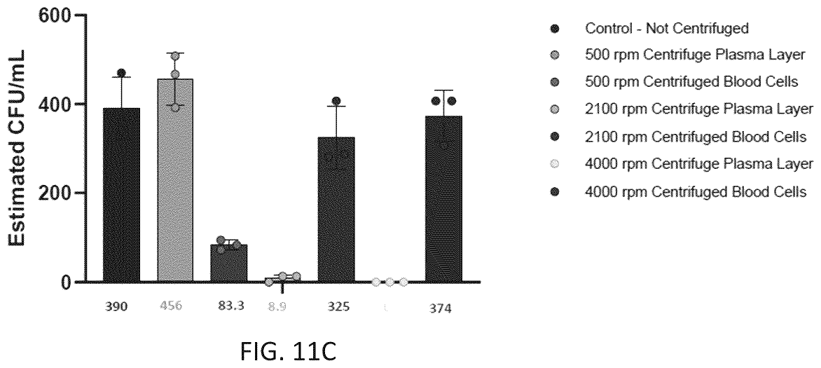

As shown in FIGS. 11A-11B, RBCs were fully recovered from centrifuges patient samples. For example, RBC recovery was measured in freshly drawn EDTA whole blood samples after centrifugation. The samples were centrifuged at 2100 rpm for 10 min or at 4200 rpm for 60 min to separate RBCs in blood pellets from supernatant plasma as shown in FIG. 9A. As shown in FIG. 9B, both centrifuge conditions gave similar volumes of plasma/RBC concentrated sample—approximately 5 mL each. These results demonstrate that clinically relevant blood samples can be pre-concentrated twice with minimal or no loss of RBCs. The pre-concentration can lead to reduced processing times of clinical diagnostics.

Bacteria Recovery from Centrifuged Samples

As shown in FIG. 11C, bacteria samples were fully recovered from centrifuged patient samples. For example, samples centrifuged at 0, 500 rpm, and 4000 rpm contained around 390, 456, and 374 CFU/ml bacteria. These results demonstrate that clinically relevant blood samples can be pre-concentrated with minimal or no loss of bacteria. The pre-concentration can lead to reduced processing times of clinical diagnostics.

For experiments, fresh EDTA whole blood samples of approximately 10 mL volume were used. The samples were spiked with 200 μL of E. coli K-12. Sample bacterial density was estimated optically with OD600 being 1.75 and dilution of 1:113,500. For CFU concentration estimation a standard curve was used (CFU/mL=1.36×10⁹×OD600−3.26×10⁶). Pre-centrifuged blood/bacteria mixture was streaked on Luria-Bertani (LB) agar of 100 μL volume. It was estimated that approximately 4200 bacteria were spiked into approximately 10 mL blood. Blood samples were centrifuged at three different speeds after separating into approximately 3 mL aliquots: (1) 10 min at 500 rpm; approximately 1.2 mL plasma separated from approximately 1.8 mL blood pellet; (2) 10 min at 2100 rpm; approximately 1.5 mL plasma separated from approximately 1.5 mL blood pellet; and (3) 15 min at 4000 rpm; approximately 1.5 mL plasma separated from approximately 1.5 mL blood pellet. The samples from the plasma layer and the pellet layer were streaked on 100 μL LB agar to count CFU and estimate concentration.

### Label-Free Microscopy

Bacteria in blood samples will be tested for antimicrobial susceptibility (AST) and identification (ID) on an optical setup that combines label-free microscopy techniques such as quantitative phase microscopy (QPM), instant structured illumination microscopy (iSIM), and hyperspectral imaging (HSI).

### Example Workflow

An example workflow for sample preparation and analysis is as follows. Whole blood with suspected infectious agent (e.g., bacteria and/or fungus) is diluted with a lysing composition (e.g., in a 1:5 ratio, in a 1:10 ratio). The mixture is incubated (e.g., 25° C. or 37° C.) for a designated time point (e.g. 15 minutes or 30 minutes). Viability is assessed by streaking a representative volume of blood prior to incubation (t=0) and at the final time point on agar plates and counting the CFU recovered.

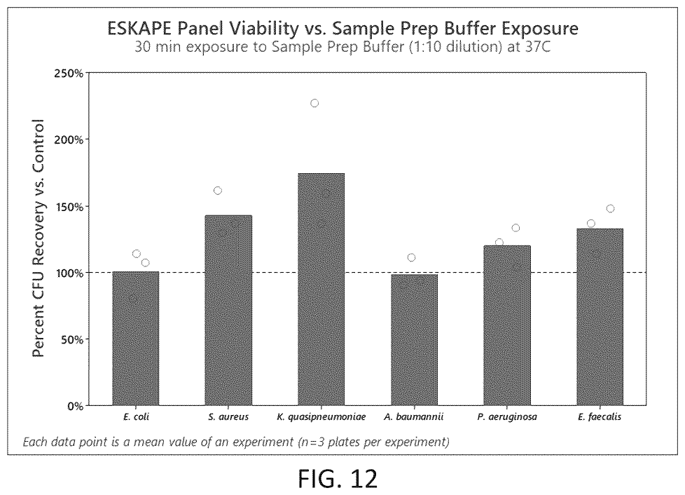

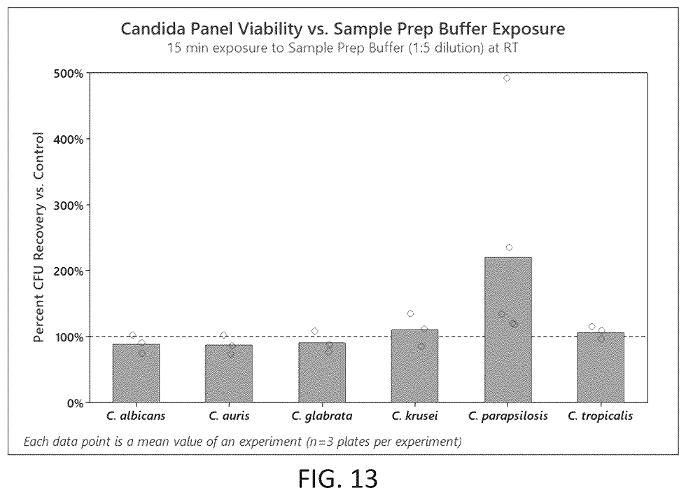

This workflow was tested for various lysis compositions that included different combinations of one or more surfactants, including of saponin, a poloxamer, Triton X-100, and IGEPAL CA-630, and, in some cases, one or more enzymes. As shown in FIG. 12, representative bacteria remain largely viable (>95%) after exposure to such a lysing composition in a 1:10 dilution ratio at 37° C. for 30 minutes. As shown in FIG. 13, representative Candida spp. remain largely viable (>85%) after exposure to such a lysing composition in a 1:5 dilution ratio at room temperature for 15 minutes. These dilutions, time points, and temperatures are examples and not inclusive of all possible combinations that may be used in this workflow.

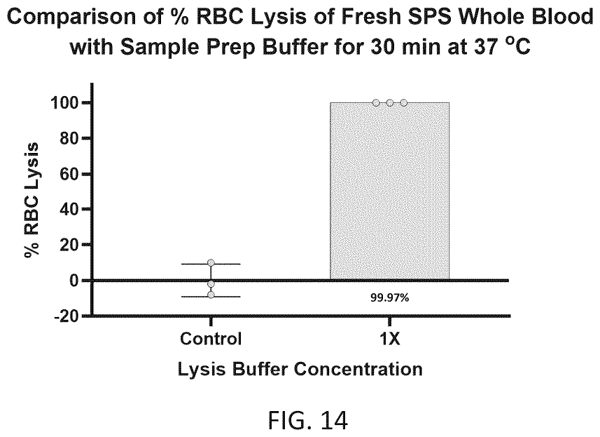

As shown in FIG. 14, sodium polyanethole sulfonate (SPS) whole blood samples exposed 30 minutes at 37° C. to a lysing composition resulted in 99.97% RBC lysis.

In this application, unless otherwise clear from context or otherwise explicitly stated, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; (iv) the terms “about” and “approximately” may be understood to permit standard variation as would be understood by those of ordinary skill in the relevant art; and (v) where ranges are provided, endpoints are included. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

It is contemplated that systems, devices, methods, and processes of the disclosure encompass variations and adaptations developed using information from the embodiments described herein. Adaptation and/or modification of the systems, devices, methods, and processes described herein may be performed by those of ordinary skill in the relevant art.

Throughout the description, where compositions and systems are described as having, including, or comprising specific components, or where processes and methods are described as having, including, or comprising specific steps, it is contemplated that, additionally, there are compositions and systems according to certain embodiments of the present disclosure that consist essentially of, or consist of, the recited components, and that there are processes and methods according to certain embodiments of the present disclosure that consist essentially of, or consist of, the recited processing steps.

It should be understood that the order of steps or order for performing certain action is immaterial so long as operability is not lost. Moreover, two or more steps or actions may be conducted simultaneously. As is understood by those skilled in the art, the terms “over”, “under”, “above”, “below”, “beneath”, and “on” are relative terms and can be interchanged in reference to different orientations of the layers, elements, and substrates included in the present disclosure. For example, a first layer on a second layer, in some embodiments means a first layer directly on and in contact with a second layer. In other embodiments, a first layer on a second layer can include another layer there between.

Headers are provided for the convenience of the reader and are not intended to be limiting with respect to the claimed subject matter.

Certain embodiments of the present disclosure were described above. It is, however, expressly noted that the present disclosure is not limited to those embodiments, but rather the intention is that additions and modifications to what was expressly described in the present disclosure are also included within the scope of the disclosure. Moreover, it is to be understood that the features of the various embodiments described in the present disclosure were not mutually exclusive and can exist in various combinations and permutations, even if such combinations or permutations were not made express, without departing from the spirit and scope of the disclosure. The disclosure has been described in detail with particular reference to certain embodiments thereof, but it will be understood that variations and modifications can be effected within the spirit and scope of the claimed invention.

1. A method comprising contacting a blood sample comprising one or more infectious agents to a lysing composition thereby obtaining a lysed blood composition comprising the one or more infectious agents.

2. The method of claim 1, wherein the one or more infectious agents comprises a bacteria.

3. (canceled)

4. The method of claim 1, wherein the one or more infectious agents comprises one or more fungi.

5-6. (canceled)

7. A method comprising contacting a blood sample comprising bacteria to a lysing composition thereby obtaining a lysed blood composition comprising the bacteria.

8-10. (canceled)

11. The method of claim 1, wherein the lysing composition comprises lysing agent comprising IGEPAL CA-630, saponin, sodium cholate, Triton X-100, or one or more poloxamers.

12-24. (canceled)

25. The method of claim 1, wherein the lysing composition comprises lysing agent comprising two or more surfactants.

26-27. (canceled)

28. The method of claim 25, wherein the two or more surfactants comprise a first surfactant having a concentration in a total volume of the lysing composition and the blood sample when the lysing composition and the blood sample are first contacted in a range of from 0.01% to 0.3% and a second surfactant having a concentration in the total volume in a range of from 0.4% to 5% and, optionally, a third surfactant having a concentration in the total volume in a range of from 0.25% to 5%.

29. (canceled)

30. The method of claim 28, wherein the first surfactant is a detergent, the second surfactant is a detergent, or both the first surfactant and the second surfactant are detergents.

31. The method of claim 28, wherein the second surfactant is a saponin and the first surfactant is Triton X-100.

32. The method of claim 28, wherein the first surfactant is Triton X-100 and the second surfactant is a poloxamer.

33. The method of claim 25, wherein a concentration of the lysing agent in a total volume of the lysing composition and the blood sample when the lysing composition and the blood sample are first contacted is at least 0.4% and no more than 3%.

34-35. (canceled)

36. The method of claim 1, wherein the lysing composition comprises one or more enzymes.

37. The method of claim 36, wherein the enzyme is DNase I, RNase, Proteinase K, one or more lipases, or one or more phospholipases.

38. The method of claim 36, wherein a concentration of each of one or more enzymes in a lysing composition is no more than 50 U/mL.

39-49. (canceled)

50. The method of claim 1, wherein a total concentration of surfactants in a total volume of the lysing composition and the blood sample when the lysing composition and the blood sample are first contacted is no more than 3%.

51-55. (canceled)

56. The method of claim 1, wherein the step of contacting the blood sample to the lysing composition comprises lysing at least 80% of red blood cells in the blood sample.

57-60. (canceled)

61. The method of claim 1, wherein the blood sample is a whole blood sample.

62-63. (canceled)

64. The method of claim 1, wherein, before the contacting, the blood sample has been treated with an anticoagulant, wherein the anticoagulant is ethylenediaminetetraacetic acid (EDTA), sodium polyanethole sulfonate (SPS), sodium citrate, or lithium heparin.

65-81. (canceled)

82. The method of claim 1, wherein at least 50% of colony-forming units in the lysed blood composition are preserved.

83-84. (canceled)

85. The method of claim 1, wherein the one or more infectious agents in the lysed blood composition are viable.

86-92. (canceled)

93. The method of claim 1, wherein the lysed blood composition has been formed from a combination of the blood sample and the lysing composition in a ratio in a range of from 3:1 to 1:10.

94. The method of claim 1, wherein the step of contacting the blood sample and the lysing composition comprises combining the blood sample and the lysing composition such that the blood sample is at least 75% of total volume thereof.

95-136. (canceled)

137. The method of claim 28, wherein the second surfactant is a saponin and the first surfactant is IGEPAL CA-630.

138. The method of claim 28, wherein the second surfactant is sodium cholate and the first surfactant is Triton X-100.

139. The method of claim 1, wherein the lysing composition comprises lysing agent comprising a saponin and IGEPAL CA-630.

140. The method of claim 1, wherein the lysing composition comprises lysing agent comprising sodium cholate and Triton X-100.
